(19) United States
(12) Patent Application Publication
(10) Pub. No.: US 2024/0277590 A1
(43) Pub. Date: Aug. 22, 2024

(54) COMPOSITIONS AND METHODS FOR STYLING HAIR FIBERS

(71) Applicant: LANDA LABS (2012) LTD., Rehovot (IL)

(72) Inventors: Sagi ABRAMOVICH, Ra'anana (IL); Tamar ASHER, Kiryat Ono (IL); Nir KOJOKARO, Rehovot (IL); Yishai KARTON, Nes Ziona (IL); Alexander BLUVSTEIN, Rishon LeZion (IL)

(73) Assignee: LANDA LABS (2012) LTD., Rehovot (IL)

(21) Appl. No.: 18/444,693

(22) Filed: Feb. 18, 2024

Related U.S. Application Data
(63) Continuation-in-part of application No. PCT/IB2022/057741, filed on Aug. 18, 2022.

(30) Foreign Application Priority Data
Aug. 19, 2021 (GB) 2111904.5

(51) Int. Cl. A61K 8/34; A61Q 5/06

(52) U.S. Cl. CPC A61K 8/347; A61Q 5/06; A61K 2800/81; A61K 2800/882

(57) ABSTRACT

The present disclosure relates to a hair styling composition comprising a phenol-based monomer and a water-soluble hygroscopic agent capable of penetrating within the hair upon application thereto, the curing of the monomers adapted to internally form a polymer able to overcome the tendency of the hair to revert to its native shape. When curing is performed while the hair is in a desired modified shape, the resulting polymers may maintain the modified shape, whilst the hygroscopic agents may prolong the hair styling effect. Kits allowing to prepare the hair styling composition and methods of using the same are also disclosed.

### CROSS-RELATED TO RELATED APPLICATIONS

The present application is a Continuation-in-Part Application of International Application No. PCT/IB2022/057741, filed on Aug. 18, 2022, which claims Paris Convention priority from Great-Britain Patent Application No. GB 2111904.5, filed on Aug. 19, 2021. The entire contents of the afore-mentioned applications are incorporated herein by reference for all purposes as if fully set forth herein.

### FIELD

The present disclosure relates to compositions, kits, and methods for styling keratinous fibers, such as mammalian hair.

### BACKGROUND

The mammalian (e.g., human) hair fiber is a layered structure, wherein the outermost layer is the cuticle, a thin protective layer made of keratin protein, surrounding a central hair shaft composed of a cortex and a medulla. The cuticle layer is built from scale-shaped cells, layered one over the other in an overlapping manner, similarly to shingles on a roof. The physical appearance and the shape of hair fibers are determined by a variety of interactions between the keratin chains within the fibers, the amino acid composition of the keratin being responsible for the types of possible interactions. Cysteine side chains allow for the formation of disulfide bonds, while other amino acids residues may form weaker interactions such as hydrogen bonds, hydrophobic interactions, ionic bonds, Coulombic interactions etc. The presence of such reactive groups in the fiber, their proportion along the fiber as well as their availability due to the fiber conformation, determine the occurrence of these interactions and the appearance of the fiber or of the hair constituted by a plurality of such fibers.

The disulfide covalent bonds that may form between two thiol side-chains of two adjacent cysteine residues account for the fibers' structure stability, durability and mechanical properties, and the breaking of these bonds by various procedures is the mechanism behind most contemporary methods of permanent hair styling (mainly straightening or waving).

One such procedure, termed “Japanese straightening”, involves reductive agents, e.g., mercaptans or sulfites, which selectively cleave the disulfide bonds, whereby the keratins mechanically relax, followed by re-oxidation of the free sulfhydryl groups, allowing for the recombination of the disulfide bonds at the end of the process, while the hair is at the conformation adapted to achieve the desired styling. Various styling means, such as hot iron or hair dryer, can be used to induce additional stress to permanently conform the hair to the desired conformation (whether straight or wavy).

Another procedure for permanent styling of the hair relies on even harsher reductive agents, such as strong alkaline agents at pH higher than 11.0. Under these conditions, the disulfide bonds are cleaved in a less selective manner when the alkaline agents deeply permeate into the pH-induced swelled hair, disrupting possible rearrangement of the disulfide bonds.

Other procedures, termed “keratin straightening” and “organic straightening”, and including “Brazilian straightening”, are considered semi-permanent, and involve the massive use of aldehydes, namely, formaldehyde, formaldehyde-producing agents, or glutaraldehyde, most straightening products containing 2-10% of such chemicals. Exemplary formaldehyde-producing agents, also referred to as formaldehyde-releasing agents, include glyoxylic acid and its derivatives (e.g., glyoxyloyl carbocysteine), some of them being commonly used as preservatives. These aldehyde-based or -producing agents react with the keratin in the hair-fibers, acting as cross-linkers, thus prolonging the durability of the new hair conformation and shape. Formaldehyde and glutaraldehyde are considered carcinogenic, and can cause eyes and nose irritation, as well as allergic reactions of the skin, eyes, and lungs. They are therefore considered hazardous by the Occupational Safety and Health Administration (OSHA), and hair styling products manufacturers are required to comply with a limit of 0.2 weight by weight percentage (wt. %) or less of these materials, some jurisdictions even requiring 0.1 wt. % or less, by weight of the composition. OSHA tested several keratin treatments and found that many of the products contained formaldehyde in the solution or emitted formaldehyde fume with heating even though the products were marketed as “formaldehyde free” or did not include formaldehyde in the list of ingredients, leaving the community in doubt concerning the claimed safety of such “non-formaldehyde” containing keratin-straightening products. Reports suggest that formaldehyde may simply be replaced by formaldehyde-producing agents in such products. While such products may to some extent penetrate the hair fibers under their cuticles, they are believed to mainly act by superficial coating, this external protective sheath underlying the smooth and shiny effect provided by this method. This is however a temporary effect, the coating depriving the hair of moisture, leading to brittleness, dryness and dullness of hair upon thinning of the protective keratin containing coat.

Some permanent or semi-permanent straightening methods require the use of dedicated shampoos to maintain their effect over time, such products being adapted to the particular chemical reaction each such treatment may rely on to affect hair shape. In addition, such methods show little flexibility if one wishes to further change a hair color, a hair style or to revert to the natural style, such steps typically requiring either conducting a new permanent treatment, further damaging the hair, or waiting for regrowth of hair.

The amino acids making up the keratin protein of hair fibers also contain side-chains capable of forming non-covalent weaker bonds, such as hydrogen bonds that may form between polar and/or charged side chains in the presence of water molecules. Such hydrogen bonds can form between the amino acids on the outer surface of the cuticle scales, as well as on the internal part of the scales or beneath them. Breaking of these hydrogen bonds upon exposure of the hair to heat (e.g., by a flat iron or a hair dryer, thus allowing removal of the water from the hair), and their reformation by drying or cooling, provides for temporary hair styling. While such methods do not involve reagents damaging to the hair, their effect is transient, due to the sensitivity of the fibers so shaped to water, including to ambient relative humidity.

The classification of hair styling methods between permanent, semi-permanent or temporary typically depends on the number of shampoos it may take for the hair to regain its native shape. Permanent methods may be sufficiently harsh to require growth of new hair fibers and whilst some non-transient styling may be voluntarily reversed, such methods may by themselves be damaging.

Thus, there is a need for hair styling methods, which reduce the need for hair-damaging and hazardous reagents, and advantageously may at the same time provide long-lasting style and shape for the hair.

### SUMMARY

The present disclosure relates to compositions, kits and methods comprising or using the same, for styling of hair fibers developed in order to overcome, inter alia, at least some of the drawbacks associated with traditional methods of hair styling. As used herein “styling” of hair includes any action modifying its shape in a visually detectable and desirable manner, it includes straightening or relaxing of hair, if wavy, curly or coiled; or conversely curling of hair, if the hair is relatively straighter than desired; hence any increase or decrease of the natural tendency of the hair fibers to curl.

Advantageously, the curable compositions and methods according to the present teachings allow for temporary or permanent hair styling without cleavage of disulfide bonds within the hair fiber or otherwise permanent alteration of its molecular structure. Hence, if the hair fibers have in their native (unmodified) shape prior to styling according to the present teachings a certain number of sulfur bonds, the fibers styled to have a modified shape will display essentially the same number of sulfur bonds. Alternatively, the innocuity of the present compositions and methods can be assessed by the modified hair fibers displaying essentially the same physico-chemical structure as native hair fibers. For example, in some embodiments the mechanical properties of the hair fibers are not compromised by the present compositions and methods, and some properties may even improve in particular embodiments. The fact that the chemical structure of the hair fibers is not adversely affected can be demonstrated, for example, by thermal analysis, wherein the modified and native shaped hair fibers, respectively treated or untreated by the present compositions and methods, may display at least one essentially similar endotherm temperature (as can be determined by various methods, e.g., DSC, DMA, TMA, and like methods of thermogravimetry). Endotherm temperatures of two materials or hair fibers can be considered essentially similar if within 4° C., 3° C., 2° C., or 1° C., from one another. In particular embodiments, the endotherm temperatures of the treated and untreated fibers serving as reference are measured by the same thermal analysis method, DSC being preferred.

In a first aspect of the invention, there is provided a method of styling mammalian hair fibers by modifying a shape of the fibers from a native shape to a desired modified shape, the method comprising:
a) applying to individual hair fibers a hair styling composition to cover the hair fibers, the hair styling composition comprising at least one water-insoluble phenol-based monomer (PBM), at least one water-soluble hygroscopic agent (WHA), water, optionally one or more curing facilitator miscible with the PBM, and further optionally, at least one auxiliary polymerization agent;
b) allowing the hair styling composition to remain in contact with the hair fibers for a period of time sufficient to ensure at least partial penetration of the PBM(s) and the WHA(s) into the hair fibers; and
c) applying energy to the hair fibers, so as to at least partially cure at least part of the PBMs having penetrated within the hair fibers, said partial curing optionally occurring while the hair fibers are in the desired modified shape.

The pH of the composition can be selected to facilitate the penetration of PBM and WHA into the hair fibers, said pH being different that the isoelectric point of the fibers being treated at which penetration, if any, would be minimal. In some embodiments, the pH of the hair styling composition is in a range of pH 1 to pH 3.5, or pH 5 to pH 11.

In some embodiments, prior to step a) of applying the hair styling composition comprising the PBM(s) and WHA(s), one or more of the following steps is performed:
A—the at least one PBM, and/or the at least one curing facilitator, and/or the at least one auxiliary polymerization agent are pre-polymerized prior to mixing with the water; and/or
B— the hair fibers are pre-treated by at least one of:
cleaning the hair fibers;
drying the hair fibers at a temperature and for a period of time sufficient to ensure breakage of at least part of a plurality of hydrogen bonds of the hair fibers; and
applying a pre-treating composition to the hair fibers.

In some embodiments, when the at least one PBM is pre-polymerized, optionally in the presence of other agents as afore-mentioned, the obtained composition can have a viscosity within the range of 50 to 1,000 millipascal-second (mPa·s), within the range of 70 to 800 mPa·s, within the range of 100 to 600 mPa·s, within the range of 200 to 400 mPa·s, or within the range of 300 to 400 mPa·s, as measured at 25° C. and a shear rate of about 200 sec⁻¹.

As discussed in more details in the context of restyling and de-styling, the actual styling step of providing a modified shape to the hair fibers treated by the present methods, need not necessarily be performed concomitantly with the curing of the monomers progressively forming a polymer able to overcome the tendency of the hair fibers to revert to their previous (e.g., unmodified/native/differently modified) shape. Once the polymer has formed within the hair fibers, their shape can be modified when desired at a later time. The treating method can be considered a method of styling regardless of the timeline for modifying the overall shape of the fibers, since mere formation of the polymer within the fiber may provide volume, also considered a styling effect regardless of the extent of detectability of the change.

In some embodiments, the energy applied to at least partially cure at least part of the energy curable phenol-based monomers having penetrated within the hair fibers is thermal energy, the heat being conveyed to the hair fibers by conduction (e.g., direct contact with a styling iron), by convection (e.g., using a hot air blower, hair dryer), or by radiation (e.g., using a ceramic far infrared (IR) radiation hair dryer). In other embodiments, the applied energy is more generally electromagnetic (EM), which in addition to above-mentioned IR radiation, may include for instance ultraviolet (UV) radiation. Some PBMs may be curable predominantly or solely by thermal energy (heat), while others may be curable predominantly or solely by electromagnetic energy. The former can also be referred to as heat-curable monomers, while the latter can also be referred to as EM-curable monomers. In some embodiments, the PBMs may be curable by both mechanisms, in which case they may be referred to as hybrid curable monomers.

In some embodiments, the fibers treated by the present methods and the untreated fibers (or similar corresponding ones) display at least one endotherm temperature within 4° C., within 3° C., within 2° C., or within 1° C. from one another as measured by thermal analysis.

For conciseness, the materials that may serve for the preparation of the hair styling compositions that can be applied in the present method for styling of hair are detailed hereinafter with reference to the compositions, the desired properties of their ingredients, and their relative proportions, these features applying mutatis mutandis for the sake of the methods.

In a second aspect of the invention, there is provided a method of restyling hair fibers having a hair shape being a first modified hair shape achieved by the styling methods or with the hair styling compositions being further detailed herein, the restyling method comprising:
applying energy to hair fibers having a first shape and containing in an inner part thereof a synthetic polymer having a softening temperature, the synthetic polymer being able to provide a shape to the hair fibers while at a temperature lower than its softening temperature, the application of energy being for a period of time sufficient to soften the synthetic polymer within the hair fibers; and
terminating the application of energy while the hair fibers are in a desired restyling second modified hair shape, the second modified hair shape being the same or different from the first shape.

In some embodiments, the fibers having the desired second shape display at least one endotherm temperature within 4° C., within 3° C., within 2° C., or within 1° C. from untreated fibers lacking the synthetic polymer as measured by thermal analysis.

In some embodiments, the application of thermal energy for restyling in step A-occurs for at least 5 minutes and at a temperature above the softening temperature of the polymer, for instance at a temperature of at least 50° C. In some embodiments, the temperature of restyling is sufficiently high to additionally decrease the amount of residual water within the hair fibers.

In a third aspect of the invention, there is provided a method of de-styling hair fibers having a modified hair shape achieved by the styling methods or with the hair styling compositions being further detailed herein. Namely, there is provided a method of de-styling hair fibers comprising in an inner part thereof a synthetic polymer having a softening temperature, the synthetic polymer being able to provide a shape to the hair fibers while at a temperature lower than its softening temperature, the de-styling method comprising:
a) applying energy to hair fibers having a first shape, the application of energy being for a period of time sufficient to soften the synthetic polymer within the hair fibers, so that the hair fibers are at least for 10 minutes at least 40° C., or preferably at least 45° C.;
b) applying water during the application of energy to enable at least a partial reformation of hydrogen bonds freed by the softening of the synthetic polymer; and
c) terminating the application of energy and water while the hair fibers are devoid of contrived constraint, so as to allow the polymer to regain an unsoftened form while the hair fibers are in a natural unmodified shape.

In some embodiments, the fibers having the natural unmodified shape display at least one endotherm temperature within 4° C., within 3° C., within 2° C., or within 1° C. from untreated fibers lacking the synthetic polymer as measured by thermal analysis.

The ability to restyle or de-style hair previously treated by the present methods and compositions (i.e., hair fibers including in inner parts thereof polymers synthesized in situ by cross-linking of PBMs in presence of WHA(s)) is advantageous and unexpected in the field, where traditional methods typically require applications of suitable compositions to further change hair shape.

As used herein, the term “treated” with regards to hair fibers, refers to fibers that were treated with the compositions or by the methods of the present invention, and conversely, the term “untreated” refers to hair fibers that were not treated with the compositions or by the methods herein disclosed.

Hair treated by the present methods and compositions may display additional advantages, such as with respect to the mechanical properties of the treated hair and/or with respect to the types of hair that can be treated. For instance, while conventional styling methods are typically deleterious to mechanical properties of the hair, hair fibers treated according to the present teachings may display at least one tensile property (e.g., elastic modulus, break stress and toughness of the hair fibers) which is at least equal to the same property in the corresponding untreated fibers. Additionally, or alternatively, the present methods and compositions can be applied to hair already processed by conventional hair procedures, such as bleaching or coloring, whereas conventional styling methods may be incompatible.

In a fourth aspect of the invention, there is provided a hair styling composition for modifying a shape of mammalian hair fibers, the hair styling composition being selected from:
a single-phase composition, the single phase including at least one water-insoluble phenol-based monomer (PBM), at least one water-soluble hygroscopic agent (WHA), water having a pH selected to increase a penetration of at least part of the PBM(s) and WHA(s) into the hair fibers, and a co-solvent, the single phase optionally further including one or more curing facilitators miscible therewith; and
an oil-in-water emulsion, the emulsion consisting of a) an oil phase comprising at least one water-insoluble phenol-based monomer (PBM) and optionally one or more curing facilitators miscible therewith; and b) an aqueous phase containing at least one water-soluble hygroscopic agent (WHA), and having a pH selected to increase a penetration of at least part of the PBM(s) and WHA(s) into the hair fibers;
the hair styling composition being further detailed herein and claimed in the appended claims.

In some embodiments of any of the aforesaid aspects, the hair styling composition contains less than 0.2 wt. % of small reactive aldehydes (SRA), the SRA being selected from formaldehyde, formaldehyde-forming chemicals, glutaraldehyde, and glutaraldehyde-forming chemicals by total weight of the composition. In other embodiments, the hair styling composition contains less than 0.1 wt. %, less than 0.05 wt. %, less than 0.01 wt. %, less than 0.005 wt. %, or less than 0.001 wt. % of SRAs by total weight of the composition.

The water-soluble hygroscopic agent, or each water-soluble hygroscopic agent if more than one, that can be used in embodiments of any of the aforesaid aspects is characterized by at least one of the following features:
the WHA is a polar non-electrolyte which does not substantially ionize when dissolved (e.g., in water or in an aqueous solution or phase);
the WHA is capable of forming hydrogen bonds with water molecules stronger than the hydrogen bonds that water molecules may form among themselves, in other words the WHA has a hydrogen bond energy with water of at least 21 kiloJoules per mol (kJ/mol), at least 22.5 kJ/mol, at least 25 kJ/mol, or at least 27.5 kJ/mol;
the WHA has a hydrogen bond energy with water of at most 40 kJ/mol, at most 35 kJ/mol, or at most 32.5 kJ/mol;
the WHA has a hydrogen bond energy with water in a range between 21 kJ/mol and 40 kJ/mol, between 22.5 kJ/mol and 35 kJ/mol, or between 27.5 kJ/mol and 32.5 kJ/mol;
the WHA has a solubility in water of 5 wt. % or more, 10 wt. % or more, 20 wt. % or more, or 30 wt. % or more, by weight of the water, as measured at a temperature of 25° C.;
the WHA has a solubility in water of 150 wt. % or less, 125 wt. % or less, 100 wt. % or less, or 75 wt. % or less, by weight of the water, as measured at a temperature of 25° C.;
the WHA has a solubility in water in a range between 5 wt. % and 150 wt. %, between 10 wt. % and 125 wt. %, between 20 wt. % and 100 wt. %, or between 30 wt. % and 75 wt. %, by weight of the water, as measured at a temperature of 25° C.;
the WHA has a solubility in the hair styling composition or in an aqueous phase thereof of 5 wt. % or more, 10 wt. % or more, 20 wt. % or more, or 30 wt. % or more, by weight of the composition or aqueous phase thereof, as measured at a temperature of 25° C.;
the WHA has a solubility in the hair styling composition or in an aqueous phase thereof of 140 wt. % or less, 110 wt. % or less, 80 wt. % or less, or 50 wt. % or less, by weight of the composition or aqueous phase thereof, as measured at a temperature of 25° C.;
the WHA has a solubility in the hair styling composition or in an aqueous phase thereof in a range between 5 wt. % and 140 wt. %, between 10 wt. % and 110 wt. %, between 20 wt. % and 80 wt. %, or between 30 wt. % and 50 wt. %, by weight of the composition or aqueous phase thereof, as measured at a temperature of 25° C.;
the WHA has a melting temperature Tₘ of 25° C. or more, 35° C. or more, or 45° C. or more;
the WHA has a melting temperature Tₘ of 200° C. or less, 180° C. or less, or 160° C. or less;
the WHA has a melting temperature Tₘ in a range between 25° C. and 200° C., between 35° C. and 180° C., or between 45° C. and 160° C.;
the WHA has a boiling temperature T_{b} greater than the boiling temperature of water as measured at atmospheric pressure, in other words the WHA has a boiling temperature T_{b} of 100° C. or more, 120° C. or more, or 140° C. or more;
the WHA has a boiling temperature T_{b} of 300° C. or less, 250° C. or less, or 225° C. or less;
the WHA has a boiling temperature T_{b} in a range between 100° C. and 300° C., between 120° C. and 250° C., or between 140° C. and 225° C.;
the WHA is less volatile than water, hence has a vapor pressure smaller than the vapor pressure of water, in other words the WHA has a vapor pressure of 2.3 kiloPascal (kPa) or less, and preferably of 1.0 kPa or less, 0.1 kPa or less, 10 Pa or less, or 1 Pa or less, as measured at 25° C.;
the WHA has a vapor pressure of 1 mPa or more, 10 mPa or more, or 50 mPa or more, as measured at 25° C.;
the WHA has a vapor pressure in a range between 1 mPa and 1 kPa, between 1 mPa and 0.1 kPa, between 1 mPa and 50 Pa, between 1 mPa and 10 Pa, or between 1 mPa and 1 Pa;
at a concentration of 1 wt. % or less by weight of the hair styling composition, the WHA does not substantially interfere with a pH of the hair styling composition, the pH of the hair styling composition in presence said concentration of the WHA being within half-a-log of the pH of the hair styling composition in absence of the WHA; and
the WHA is regulatorily approved for cosmetic use at the concentration intended in the hair styling composition.

In some embodiments, the WHA is a polar non-electrolyte which does not substantially ionize when dissolved (e.g., in a liquid containing or consisting of water). Without wishing to be bound by any particular theory, it is believed that such lack of ionization may assist in maintaining the respective charges of the hair styling composition and the hair fibers treated thereby. The respective charging of these species can provide for a zeta potential difference and gradient favoring the driving of the PBM(s) and WHA(s) to the hair surface, from which they may penetrate into the hair, where their polymerization can favor styling as herein taught.

In some embodiments, the WHA is a polar water-soluble hygroscopic agent fulfilling at least one of the properties recited in each of features ii) to iv) as above listed concerning its hydrogen bond energy. In some embodiments, the WHA satisfying features i) to iv) or ii) to iv) further fulfills at least one of the properties recited in each of features v) to vii) as above listed concerning its solubility in water. In some embodiments, the WHA satisfying features i) to iv), ii) to iv), i) to vii) or ii) to vii) further fulfills at least one of the properties recited in each of features viii) to x) as above listed concerning its solubility in the hair styling composition or in an aqueous phase thereof. In some embodiments, the WHA satisfying features i) to iv), ii) to iv), i) to vii), ii) to vii, i) to x) or ii) to x) is liquid at room temperature (circa 25° C.), but it can alternatively be solid and further fulfills at least one of the properties recited in each of features xi) to xiii) as above listed concerning its melting temperature. In some embodiments, the WHA satisfying features i) to iv), ii) to iv), i) to vii), ii) to vii, i) to x), ii) to x), i) to xiii), or ii) to xiii) is solid at room temperature and further fulfills at least one of the properties recited in each of features xiv) to xvi) as above listed concerning its boiling temperature. In some embodiments, the WHA satisfying features i) to iv), ii) to iv), i) to vii), ii) to vii, i) to x), ii) to x), i) to xiii), ii) to xiii), i) to xvi), or ii) to xvi) is solid at room temperature and further fulfills at least one of the properties recited in each of features xvii) to xix) as above listed concerning its vapor pressure. In some embodiments, the WHA satisfying features i) to iv), ii) to iv), i) to vii), ii) to vii, i) to x), ii) to x), i) to xiii), ii) to xiii), i) to xvi), ii) to xvi), i) to xix), or ii) to xix) is solid at room temperature and further fulfills feature xx) as above listed concerning its minimal impact on the pH of the hair styling composition. Preferably, feature xxi) concerning the regulatory status of the WHA and the suitability of its concentration in the present hair styling compositions for cosmetic use, apply to any combination of features i) to xx) or ii) to xx), and in particular to the combinations specifically envisioned in the present paragraph.

When used with respect to a liquid in which certain properties of the present materials are reported (e.g., solubility or lack thereof), the term “water” may refer to pure deionized or double distilled water having a pH of about 7 and a resistivity of 18.2 megaohm·cm⁻¹ at 25° C., as customary for the applicable measurements. Yet, as shall be clear from context, water may also refer to liquids of different grades including tap water as conventionally used in hair styling methods.

In some embodiments, the hair styling composition further comprises an auxiliary polymerization agent containing at least one functional group capable of cross-polymerization with at least one of the PBM and the curing facilitator, the functional group being selected from: a hydroxyl, a carboxyl, an amine, an anhydride, an isocyanate, an isothiocyanate and a double bond.

In some embodiments, the hair styling composition further comprises at least one additive selected from a group comprising an emulsifier, a wetting agent, a thickening agent and a charge modifying agent.

In a fifth aspect of the invention, there is provided a kit for styling mammalian hair fibers, the kit comprising:
a first compartment containing at least one water-insoluble phenol-based monomer (PBM); and
a second compartment containing at least one water-soluble hygroscopic agent (WHA) and at least one of:
water;
a co-solvent; and
a pH modifying agent;
wherein the contents of the second compartment are a liquid having a pH selected to increase a penetration of at least part of the PBM(s) and WHA(s) into the hair fibers; and
wherein mixing of the compartments produces a hair styling composition as a single-phase composition or an oil-in-water emulsion, as further detailed herein and claimed in the appended claims.

In some embodiments, the at least one PBM of the first compartment is pre-polymerized prior to its placing in the kit.

In some embodiments, the hair styling composition prepared from mixing of the kit compartments is ready to use, whereas in other embodiments, the hair styling composition needs be further diluted (e.g., with tap water) by the end-user prior to mixing of the compartments and/or application on the hair fibers.

In some embodiments, at least one curing facilitator, selected from a cross-linker (suitable for condensation- and/or addition-curing) and a curing accelerator, is further comprised within the hair styling composition, in the kit, or in a method of using the same. The compositions and the methods may, in some embodiments, comprise two or more types of cross-linkers enabling in combination both addition-curing and condensation-curing of the pre-polymers. Such a curing facilitator may be placed in the first or second compartment, when it does not spontaneously (e.g., at room temperature) react with any one of the components of the first or second compartments, respectively. Alternatively, the curing facilitator may be placed in a separate third compartment to be mixed with the first and second compartments upon preparation of the hair styling composition as a single-phase composition or an oil-in-water emulsion.

Compartments of the kits (and their respective contents) are selected so as to avoid or reduce any reaction that would diminish the efficacy of the product during storage of the kit at a desirable storing temperature (e.g., not exceeding room temperature). In some embodiments, regardless of pre-polymerization of the PBM(s) or lack thereof, the first and/or third compartments are maintained in an inert environment, preferably under an inert gas, e.g., argon or nitrogen. For similar reasons, the compartments can be selected to be opaque to radiation or sealed against any factor detrimental to the stability of their contents.

In some embodiments, the first compartment of the kit further comprises at least one auxiliary polymerization agent.

In some embodiments, the kit further comprises at least one co-solvent, which may be contained in the first, second, or a separate additional compartment.

In some embodiments, the kit further comprises at least one additive selected from a group comprising: an emulsifier, a wetting agent, a thickening agent and a charge modifying agent. When the at least one additive is oil-miscible, it may be placed in the first compartment. When the at least one additive is water-miscible, it may be placed in the second compartment. The additives can also be provided in a separate additional compartment.

Additional objects, features and advantages of the disclosure will be set forth in the detailed description which follows, and in part will be readily apparent to those skilled in the art from the description or recognized by practicing the disclosure as described in the written description and claims hereof, as well as the appended drawings. Various features and sub-combinations of embodiments of the disclosure may be employed without reference to other features and sub-combinations.

### BRIEF DESCRIPTION OF THE DRAWINGS

Some embodiments of the disclosure will now be described further, by way of example, with reference to the accompanying figures, where like reference numerals or characters indicate corresponding or like components. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the disclosure. For the sake of clarity and convenience of presentation, some objects depicted in the figures are not necessarily shown to scale.

In the Figures:

FIG. 1A is an image captured by Focussed Ion Beam milling combined with Scanning Electron Microscopy (FIB-SEM), showing a cross-section of a reference untreated hair fiber, taken at a voltage of 1.20 kV;

FIG. 1B is an image captured by FIB-SEM, showing a cross-section of the same reference untreated hair fiber of FIG. 1A, but taken at a voltage of 10 kV;

FIG. 2A is an image captured by FIB-SEM at a voltage of 1.20 kV, showing a cross-section of a hair fiber treated with an oil-in-water emulsion according to one embodiment of the present invention, before any washing cycle of the hair;

FIG. 2A′ is a schematic depiction of the FIB-SEM image of FIG. 2A;

FIG. 2B is an image captured by FIB-SEM, showing the same cross-section of the same treated hair fiber of FIG. 2A before any washing cycle of the hair, the image being taken at a voltage of 10 kV;

FIG. 2B′ is a schematic depiction of the FIB-SEM image of FIG. 2B;

FIG. 3A is an image captured by FIB-SEM at a voltage of 1.20 kV, showing a cross-section of a hair fiber treated with a same oil-in-water emulsion as shown in FIG. 2A or 2B, after 16 washing cycles of the hair;

FIG. 3B is an image captured by FIB-SEM, showing the same cross-section of the same treated hair fiber after 16 washing cycles of the hair as shown in FIG. 3A, the image being taken at a voltage of 10 kV;

FIG. 4A shows a photograph of untreated curly black hair fibers;

FIG. 4B shows a photograph of curly black hair fibers treated with a hair styling composition according to one embodiment of the present invention;

FIG. 5 shows a Differential Scanning Calorimetry (DSC) series of plots of thermal analysis of hair samples, including of a reference untreated hair sample, two hair samples treated by commercial methods, and one hair sample treated by a hypothetical innocuous composition according to one embodiment of the present invention;

FIG. 6 depicts a simplified schematic diagram of a hair styling method according to an embodiment of the present teachings;

FIG. 7A shows a photograph of hair of a volunteer having virgin hair (i.e., un-colored and un-bleached), before being treated;

FIG. 7B shows a photograph of hair of the same volunteer as shown in FIG. 7A, directly after having her hair treated with a hair styling composition according to one embodiment of the present invention; and

FIG. 7C shows a photograph of hair of the same volunteer as shown in FIG. 7B, after 18 washes following its treatment with the hair styling composition.

### DETAILED DESCRIPTION

The present disclosure relates to compositions for styling hair fibers, and more particularly to curable compositions comprising a) at least one water-insoluble phenol-based monomer (PBM) capable of undergoing polymerization by any suitable reaction that creates a macromolecule (e.g., a polymer) and b) at least one material that may extend the activity of the cured polymers, so as to prolong the hair styling durability afforded by the cured polymer. The material capable of enhancing the effect of the cured polymer is a water-soluble hygroscopic agent (WHA) able to bind water molecules. Such a WHA is believed to sequester water molecules which might have otherwise detrimentally interacted with the cured polymer, with the hair keratin and/or with any other hair constituent, in a manner affecting the constrained shape enabled by the curing of the PBMs.

Without wishing to be bound by any particular theory, the Inventors have discovered that surprisingly, the WHA(s), though being water-soluble and expected to leak out of the hair fibers readily following rinsing of treated hair or with relative ease with each shampoo, vanishing after only a few ones (e.g., less than 5, less than 4, or less than 3), are providing a protective/prolonging effect with respect to the duration of the hair styling. The Inventors posit that unexpectedly the WHAs are capable of sufficiently remaining within the hair fibers, either as isolated “water-absorbing” bodies, or in interaction with the polymers cured from the present monomers, or in interaction between the cured polymers and natural constituents of the hair fibers, or in interaction with natural constituents of the hair fibers, or in any like mechanism of action or combinations thereof. This unexpected, prolonged presence of the WHAs within the hair, in turn, reduces or delays the loss in styling effect, procured by the PBMs, otherwise normally observed over time. In other words, if a composition lacking a WHA provides for a desired hair styling resisting N washing cycles, a similar hair styling composition further including a WHA provides for a desired hair styling resisting M washing cycles, M being greater than N, all other conditions (e.g., of application) being the same.

The present invention is an improvement over international patent publication No. WO 2021/224784, of the same Applicant, the contents of which are incorporated by reference for all purposes, as if fully set herein.

As used herein, the term monomer is not meant to include only a single repeat molecule, and may include short oligomers, as long as their number of repeats yield a molecular weight not exceeding 10,000 g/mol, 5,000 g/mol, or 3,000 g/mol, as deemed suitable for the ability of any molecule (e.g., PBMs, WHAs, curing facilitators, co-solvents, etc.) to penetrate hair fibers. The hair styling compositions allow the delivery of the energy curable monomers to the inner parts of the hair fibers, together with any compound that may be required for their proper polymerization while within the fibers, such compounds being miscible with the monomers at this location. The compounds miscible with the monomers and facilitating their curing can be curing facilitators and/or co-solvents. Similarly, the hair styling compositions allow the delivery within the hair fibers of the hygroscopic agent which is preferably a polar water-soluble hygroscopic agent (WHA).

The compounds which may act within the hair fibers to promote polymerization or to reduce, delay or prevent damages to the styling effect afforded by the formed polymer can be delivered in a same phase with the monomers, or in a distinct phase. Hair styling compositions according to the present teachings can thus be single-phase compositions or oil-in-water emulsions, both typically having a pH adapted to facilitate penetration of at least part of the monomers and of any other material necessary for their suitable assembly and for the maintenance of their effects as a cured polymer. The facilitating pH may act by promoting: a) a sufficient opening of the hair scales, and/or b) a sufficient charging (e.g., as measurable by zeta potential) of the hair fibers and hair styling composition; and can be either acidic, in a range of pH 1 to pH 3.5 or pH 4, or mild acidic to mild alkaline, in a range of pH 5 to pH 8, or alkaline, in a range of pH 8 to pH 11, preferably between pH 9 and pH 11. In other words, a pH is deemed to favor penetration into the hair fibers if being in ranges other than the isoelectric point of the hair, which may slightly vary between 3.5 and 5, 4 and 5, or 3.5 and 4, depending on the hair fibers and their health status.

Methods of preparing and using these hair styling compositions and kits enabling the preparation of such compositions and hair styling therewith are also described.

The principles, uses and implementations of the teachings herein may be better understood with reference to the accompanying description and figures. Upon perusal of the description and figures present herein, one skilled in the art is able to implement the disclosure without undue effort or experimentation.

Before explaining at least one embodiment in detail, it is to be understood that the disclosure is not necessarily limited in its application to the details of construction and the arrangement of the components and/or methods set forth herein. The disclosure is capable of other embodiments or of being practiced or carried out in various ways. The phraseology and terminology employed herein are for descriptive purpose and should not be regarded as limiting. For instance, while reference is often made to head hair to illustrate the advantages of the present invention, it is clear that the present teachings would similarly apply to wigs, hair extensions, or eyelashes, to name a few alternatives. Thus, providing a durable hair style may be to hair attached to a human subject, to wigs or hair extensions, and the terminology further includes, by way of example, providing a durable eyelash shape, to eyelashes.

It is to be understood that both the foregoing general description and the following detailed description, including the materials, methods and examples, are merely exemplary of the disclosure, and are intended to provide an overview or framework to understanding the nature and character of the disclosure as it is claimed, and are not intended to be necessarily limiting.

In one aspect of the present invention, there is provided a method for styling mammalian hair fibers by modifying the shape of the fibers.

In the first step of the method of the present invention, a liquid hair styling composition is applied onto individual hair fibers, the liquid composition being a single-phase composition or an oil-in-water emulsion comprising water and: i) at least one water-insoluble phenol-based monomer (PBM) and at least one water-soluble hygroscopic agent (WHA). If the hair styling composition is provided as a single phase, a sufficient amount of a suitable co-solvent is provided to ensure the miscibility of the monomer with a water portion of the liquid, the aqueous media containing the WHA and the co-solvent being further compatible for the miscibility of any other material desired for the polymerization of the monomers (e.g., optional curing facilitators and/or auxiliary polymerization agents) or for the form and applicability of the composition (e.g., a wetting agent, a thickening agent, etc.). If the hair styling composition is provided as a bi-phasic emulsion, a co-solvent, if at all present, is provided to ensure at least the miscibility of the monomer with optional curing facilitators, the monomers being in an oil phase of the emulsion and the WHA being in an aqueous phase of the emulsion. Typically, the oil phase is dispersed as minute droplets within a continuous aqueous phase, the composition therefore forming an oil-in-water emulsion, which may additionally include an emulsifier.

Before detailing particular compounds suitable for the present methods and compositions, it is stressed that beyond the above-mentioned ability of the monomers (and of any agent facilitating their polymerization) to penetrate within the hair fibers and to be miscible with one another once and/or as long as the curing is set to proceed, the materials (including those preventing a water-induced damage to the styling effect provided by the resulting cured polymers which can be found in an aqueous phase not miscible with the monomers) need more generally to be compatible with the styling compositions, their method of preparation and their method of use. By “compatible” it is meant that the monomers, the curing facilitators, the auxiliary polymerization agents, the co-solvents, the water-soluble hygroscopic agents, or any other compatible ingredient of the present compositions, do not negatively affect the efficacy of any other compound, or the ability to prepare or use the final composition. Compatibility can be chemical, physical or both and may depend on relative amount. For illustration, a curing facilitator would be compatible if having functional groups adapted to cross-link between the monomers and/or suitable to otherwise accelerate the process. A co-solvent would be compatible if having a rate of volatility slow enough for the polymerization to proceed while the relevant materials are in a same phase. Materials would be compatible if not affected by the pH of the composition, or a temperature they might be subjected to during the preparation of the composition or its use for hair styling. While not essential, all materials could be liquid at room temperature, to facilitate preparation and use, or if solid could be readily miscible with the liquid components of the composition (e.g., a WHA solid at room temperature could readily dissolve in water or any other aqueous medium). Moreover, materials liquid at room temperature are believed to provide an improved hair feel as compared to solid materials. If a material is solid at room temperature and its dissolution requires heating, its melting point should be low enough for the temperature of heating adapted to selectively enhance its dissolution, without prematurely triggering curing of heat curable monomers or otherwise affect their ability to polymerize. If necessary, a plasticizer can be included to maintain the hair styling composition, in particular the monomers and any other curable ingredients due to penetrate the hair fibers, liquid at room temperature.

Reverting to the pre-requisite of such compounds being able to penetrate within the hair fibers, typically following suitable opening of the hair scales, without wishing to be bound by any particular theory, it is believed that smaller molecules may more easily migrate into the fibers than larger ones. While the physical size of molecules may depend on additional factors (such as special conformation and “compactness”, or lack thereof), the molecular weight of a compound may assist estimating its ability to penetrate the fibers. In some embodiments, the materials due to polymerize within the hair fibers (e.g., the monomers and cross-linkers) or due to facilitate such polymerization (e.g., the auxiliary polymerization agents, the co-solvents and curing accelerators) or due to reduce, delay or prevent damages to the formed polymer or its effect (e.g., the WHAs) have an average molecular weight (MW) of no more than 10,000 g/mol, no more than 5,000 g/mol, no more than 3,000 g/mol, no more than 2,500 g/mol, no more than 2,000 g/mol, no more than 1,500 g/mol, or no more than 1,000 g/mol.

The molecular weight of molecules having a known chemical formula can be calculated based on the molecular mass of its constituting atoms, in which case the average molecular weight is simply the molecular weight assigned to the specific molecule. For compounds formed of unknown or diverse chemical formulae, such as polymers, the average molecular weight of the population of related molecules can be provided by the supplier of the material, or independently determined by standard methods, such as high pressure liquid chromatography (HPLC), size-exclusion chromatography, light scattering, gel permeation chromatography (GPC), or matrix-assisted laser desorption/ionization time-of-flight mass spectroscopy MALDI-TOF MS, and some of these methods are described in ASTM D4001 or ISO 16014-3. Average molecular weight can then be estimated by number or by weight, both being encompassed herein.

In one embodiment, the at least one PBM is of formula I:

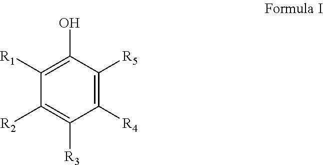

wherein
R₁, R₂, R₃ and R₅ are each independently a hydrogen atom, a hydroxyl, a linear, cyclic or branched, substituted or unsubstituted, C₁-C₂₀ alkyl, C₁-C₆ alkoxy, C₁-C₆ allyl, C₁-C₈ aromatic ester (e.g., C₁-C₈ phenyl ester), or C₁-C₈ non-aromatic ester (e.g., C₁-C₈ glycol ester); and
R₄ is a hydrogen atom, provided that R₁, R₂, R₃ and R₅ are not all hydrogen atoms, a hydroxyl, or a saturated or unsaturated C_{X}H_{Y} alkyl, wherein X is an integer equal to or less than 15, and Y is equal to 2X+1-n, n being selected from 0, 2, 4 and 6.

In some embodiments, R₁, R₂, R₃ and R₅ are each independently a hydrogen atom, hydroxyl, methyl, 2-propenyl, phenyl acetate, phenyl carboxylate, ethylene glycol monoacetate, ethylene glycol monocarboxylate, or methoxy. In other embodiments, R₄ is a hydrogen atom (provided that R₁, R₂, R₃ and R₅ are not all hydrogen atoms), hydroxyl, or a C₁₅H₃₁₋ₙ alkyl, wherein n is selected from 0, 2, 4 and 6.

In embodiments, wherein the composition includes at least one PBM of Formula I, wherein R₄ is hydroxyl and R₁, R₂, R₃ and R₅ are all hydrogen atoms, the monomer namely being benzene-1,3-diol, also known as resorcinol, then the composition may need to further include at least a second PBM of Formula I, the second PBM being other than resorcinol, and optionally a curing facilitator.

In some embodiments, the at least one PBM is selected from any of a following formulae II to V:

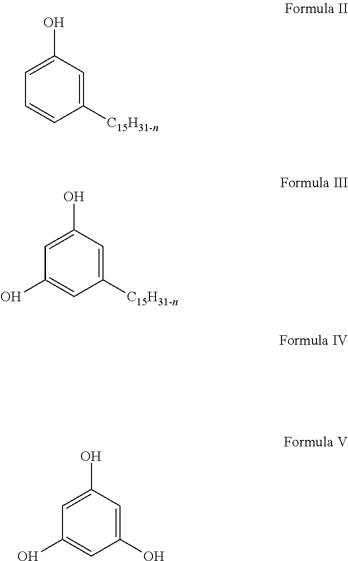

The C₁₅H₃₁₋ₙ side-chain of the PBM is a hydrocarbon (alkyl) substituent of varying degrees of unsaturation, namely can be a saturated (n=0), monoene (n=2), diene (n=4), and triene (n=6) hydrocarbon side chain.

In some embodiments, the compound of Formula II which constitutes at least one of the PBM of the present composition is a cardanol derivative. Such derivative can be selected from the group consisting of 3-pentadecylphenol (n=0), 3-[pentadec-8-enyl]phenol (n=2), 3-[penta-deca-8,11-dienyl]phenol (n=4), 3-[pentadeca-8,11,14-trienyl]phenol (n=6), and conformers thereof.

In other embodiments, the compound of Formula III which constitutes at least one of the PBM of the present composition is a cardol derivative. Such derivative can be selected from the group consisting of 5-pentadecylbenzene-1,3-diol (n=0), 5-[pentadec-8-enyl]benzene-1,3-diol (n=2), 4-[pentadeca-8,11-dienyl]benzene-1,3-diol (n=4), 5-[pentadeca-8,11-dienyl]-benzene-1,3-diol (n=4), 5-[pentadeca-9,12-dienyl]-benzene-1,3-diol (n=4), 5-[pentadeca-8,11,14-trienyl]benzene-1,3-diol (n=6), and conformers thereof.

In yet other embodiments, the compound of Formula IV which constitutes at least one of the PBM of the present composition is a 2-methyl cardol derivative. Such derivative can be selected from the group consisting of 2-methyl-5-pentadecylbenzene-1,3-diol (n=0), 2-methyl-5-[pentadec-8-enyl]benzene-1,3-diol (n=2), 2-methyl-5-[pentadeca-8,11-dienyl]benzene-1,3-diol (n=4), 2-methyl-5-[pentadeca-8,11,14-trienyl]benzene-1,3-diol (n=6), and conformers thereof.

In some embodiments, the at least one PBM of the present composition is cashew nut shell liquid (CNSL) or a component thereof.

CNSL occurs as a dark, viscous and oily liquid in the shell of the cashew nut, and it is obtained as a by-product during the industrial processing of the nut. The components of CNSL are the phenolic compounds of Formulae II-IV described above, wherein the R₄ side-chains is of varying degrees of non-conjugated unsaturation at a position or positions selected from at least one of 8^{th}, 11^{th} or 14^{th} carbon of the hydrocarbon side-chain, as depicted below:

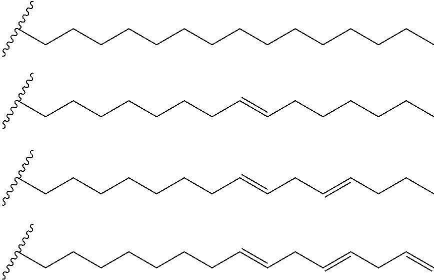 [SMILES: *CCCCCCCCCCCCCCC ; *CCCCCCC/C=C/CCCCCC ; *CCCCCCC/C=C/C/C=C/CCC ; *CCCCCCC/C=C/C/C=C/CC=C]

Natural CNSL also contains anacardic acid, represented by Formula VI:

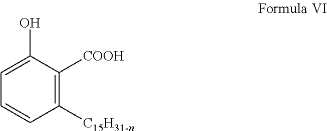

wherein the C₁₅H₃₁₋ₙ side-chain is as described above for the other components of CNSL. The amount of anacardic acid in naturally occurring CNSL is between 60 and 70 wt. %. However, technical or commercial-grade CNSL contains less than 1 wt. % anacardic acid, since it decarboxylates during the CNSL processing, and converts mainly to cardanol (Formula II). In particular embodiments, the CNSL used in the present invention contains less than 0.5 wt. %, less than 0.3 wt. %, less than 0.2 wt. % or less than 0.1 wt. % anacardic acid.

The saturated and unsaturated derivatives of each one of the CNSL constituents can be present in varying amounts. For example, the cardanol within the CNSL can be composed of 60 wt. % of the monoene derivative, 10 wt. % of the diene derivative and 30 wt. % of the triene derivative. Measurements of the amounts of these derivatives can be done using methods such as molecular distillation, Thin Layer Chromatography (TLC)/Gas-Liquid Chromatography (GLC), TLC-mass spectrometry etc.

In some embodiments, the at least one PBM is of formula VII:

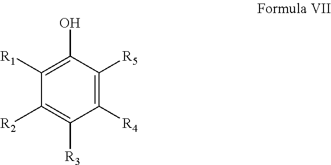

wherein:
at least one of R₁, R₂, and R₃ is a carboxylate substituent formed of a linear, branched or cyclic, substituted or unsubstituted, C₁-C₈ aromatic ester or C₁-C₈ non-aromatic ester, any of R₁, R₂, and R₃ not being a carboxylate (also referred to as a non-carboxylate R₁, R₂, or R₃) being a hydrogen atom or a hydroxyl group; and
ii) R₄ and R₅ are each independently a hydrogen atom or a hydroxyl group.

PBMs of formula VII having one hydroxyl group and one carboxylate attached to the aromatic ring can be considered derivatives of hydroxybenzoic acid, the two groups being in ortho, meta, or para position one with respect to the other.

In some embodiments, the at least one PBM of the hair styling composition is an ortho-hydroxybenzoic acid derivative, wherein the carboxylate substituent is R₁, such PBMs being known as 2-hydroxybenzoates or salicylates, which may be alkyl- or aryl-substituted salicylates. Such salicylates can be selected from a group comprising amyl salicylate, benzyl salicylate, 2-ethylhexyl salicylate, 4-tert-butylphenyl salicylate, cyclohexyl salicylate, methyl salicylate, hexyl salicylate, octyl salicylate, phenyl salicylate, salicin, and salsalate. In some embodiments, the at least one PBM of the hair styling composition is a meta-hydroxybenzoic acid derivative, wherein the carboxylate substituent is R₂, the PBM being selected from a group comprising methyl 3-hydroxybenzoate and phenyl 3-hydroxybenzoate. In some embodiments, the at least one PBM of the hair styling composition is a para-hydroxybenzoic acid derivative, wherein the carboxylate substituent is R₃, the PBM being selected from a group comprising benzyl 4-hydroxybenzoate, butyl 4-hydroxybenzoate, ethyl 4-hydroxybenzoate, heptyl 4-hydroxybenzoate, methyl 4-hydroxybenzoate, phenyl 4-hydroxybenzoate, isopropyl 4-hydroxybenzoate and N-propyl 4-hydroxybenzoate.

While the afore-said PBMs have been named by a nomenclature suitable for the presence of a single hydroxyl group on the aromatic ring of formula VII, this should not be construed as limiting and, in some embodiments, the hydroxybenzoic ring of the PBM can be further substituted by one or more hydroxyl groups, the relative positioning on the ring of the two or more hydroxyl groups with respect to the carboxylate substituent being selected from 2,3-dihydroxy-benzoates; 2,4-dihydroxy-benzoates; 2,5-dihydroxy-benzoates; 2,6-dihydroxy-benzoates; 3,4-dihydroxy-benzoates; 3,5-dihydroxy-benzoates; 2,3,4-trihydroxy-benzoates; 2,4,6-trihydroxy-benzoates; and 3,4,5-trihydroxy-benzoates; the carboxylate group being deemed in position 1 on the ring.

Furthermore, the carboxylate group formed of a linear, branched or cyclic C₁-C₈ aromatic or non-aromatic ester can be further substituted by a hydroxyl or an amine group along the side chain.

In particular embodiments, the at least one PBM of the present hair styling composition is a PBM of formula VII selected from a group comprising phenyl 2-hydroxybenzoate (or phenyl salicylate), benzyl salicylate, phenyl 3-hydroxybenzoate, phenyl 4-hydroxybenzoate, hexyl salicylate, 2-hydroxyethyl salicylate, 2-ethylhexyl salicylate, phenyl 2,3-dihydroxybenzoate, phenyl 2,4-dihydroxybenzoate, phenyl 2,5-dihydroxybenzoate, phenyl 2,3,4-trihydroxybenzoate, and phenyl 3,4,5-trihydroxybenzoate.

The hydroxyl (—OH) group(s) of the PBM, along with the varying degrees of unsaturation in the side-chains attached to the benzene aromatic ring, if other than a hydroxyl group or saturated hydrocarbon, make the PBM a highly polymerizable substance, capable of a variety of polymerization reactions (e.g., via condensation or addition). Without wishing to be bound by theory, it is believed that the PBM is capable of polymerization by condensation of its hydroxyl groups with other condensation-polymerizable groups, whereas the unsaturation of suitable side-chains can be the basis for addition polymerization, under appropriate conditions.

The PBMs previously described and further detailed herein are typically oily in nature, i.e., substantially not miscible in water, and thus, in absence of suitable amounts of appropriate co-solvents, are present in the oil phase of an oil-in-water emulsion. In some embodiments, the residual solubility of the PBMs (or of any other material deemed water-insoluble) is of 5 wt. % or less, 4 wt. % or less, 3 wt. % or less, 2 wt. % or less, 1 wt. % or less or 0.5 wt. % or less, with respect to the weight of pure water, and more appropriately with respect to the weight of the aqueous environment wherein they are to be disposed at the pH of the liquid. In other words, one weight part or less of material would dissolve in twenty weight parts of the liquid (e.g., less than 5 g of material per 100 g of water, the total amount of the mix being of 105 g and the wt. % of the material in the entire composition being of about 4.76%). Solubility can be assessed by the naked eye, the soluble composition (e.g., a single-phase composition) being typically clear (not turbid) at room temperature. This matter can alternatively be quantified by measuring the refractive index of the solution, comparing it to a calibration curve with known amounts of PBMs in water.

While water-insoluble PBMs according to the present teachings typically form hair styling compositions being oil in water emulsions, it is possible, in presence of suitable amounts of appropriate co-solvents (e.g., above 30 wt. %) to alternatively form a single-phase composition.

It is believed that the water insolubility of the compounds polymerizable within the hair fibers is pivotal to their long-lasting styling effect. Water-soluble saccharides (e.g., sucrose) that have been suggested as prospective cross-linkers for keratin, when previously modified to bear aldehyde groups allowing Shiff base formation with pending amine groups of the hair protein, were shown to be too hydrophilic/polar to provide for a wash-resistant straightening effect. For example, hair fibers treated with sucrose tetra-aldehyde as described by Patil N. V. et al. in “Natural ‘Green’ Sugar-Based Treatment for Hair Styling”, Fibers, vol. 10(2), p13-26, 2022, could not remain fully straighten after four shampoos only, despite being maintained in a humidity-controlled environment.

In some embodiments, in order to enhance the polymerization, the hair styling composition (e.g., single phase or oil-in-water emulsion) adapted to the present hair styling method further comprises, in addition to the at least one PBM: ii) at least one curing facilitator, selected from a cross-linker and a curing accelerator. Cross-linkers refer to compounds that actively participate in the curing process, and are integrated in the resulting polymer network, while curing accelerators may alternatively, or additionally, catalyze or activate the curing (e.g., by lowering the polymerization temperature or increasing its rate). Curing facilitators should preferably be oil miscible to be in a same phase as the oily monomers during their polymerization within the hair fibers. Yet, if curing accelerators are used after the application of a hair styling composition to the hair, the curing accelerators to be used in such a step can be water-soluble, assuming that the accelerating solution is aqueous.

In some embodiments, the cross-linkers can react with the monomers via a condensation-curing mechanism and may be referred to as “condensation-curable cross-linkers”. In other embodiments, the cross-linkers can react with the monomers via an addition-curing mechanism and may be referred to as “addition-curable cross-linkers”. In some embodiments, a same curing facilitator can act both as a cross-linker (incorporating the polymeric network) and as a curing accelerator (catalyzing its formation). Regardless of the type of monomers and curing facilitators that may cross-link to form within the hair fiber a network able to constrain the fibers in a desired modified shape, the resulting polymer internally formed can also be referred to as a synthetic skeleton. This term is not meant to imply that the monomers are necessarily artificial (not naturally occurring), but that the resulting polymer is synthesized in situ, and not naturally occurring within hair fibers. Simply presented, the extraneous polymer is able to “lock” the hair fibers in the desired shape, overcoming the innate force of the fibers otherwise allowing them to have or regain their natural shape. This “mechanical” metaphor is not intended to rule out any other or additional (e.g., chemical) mechanism of action of the polymers, enabling them to maintain any desired styling effect or shape. For instance, the polymers may alternatively or additionally act as a water-barrier, preventing, reducing or delaying the migration of water molecules from the external environment to the innermost keratin protein. Water molecules undesirably reaching hair constituents styled by the present method may restore hydrogen bonds within such proteins to an extent allowing the hair fibers to gradually revert to their native shape.

In some embodiments, the cross-linkers suitable for the hair styling compositions and methods of the present invention have two or more cross-linking functions, the presence of only two cross-linking functions leading to chain extension of the polymers, a process which may additionally, or alternatively, occur in absence of a cross-linker if the PBMs include such linking functions in their chemical formulae. When a polymeric network having a relatively high cross-linking density is desired, and cross-linkers are included in the composition to that effect, they advantageously have three or more cross-linking functions to increase the density of the three-dimensional network formed therewith. In some embodiments, the cross-linkers are multi-functional, having 4 or more, 5 or more, 6 or more, 7 or more, or 8 or more cross-linking functions, such functions typically not exceeding a presence of 10 per molecule of cross-linker. Additionally, or alternatively, a relatively higher cross-linking density can be obtained by using a relatively higher concentration of cross-linkers (or a higher ratio of cross-linkers to PBMs). Polymers formed by curing of the PBMs within the hair fibers with a relatively high cross-linking density are expected to form stronger skeletons for the hair styled therewith than counterparts having a relatively lower cross-linking density. Yet, the present Inventors have discovered that in some circumstances polymers formed with a relatively low cross-linking density can also be suitable. This is the case in particular when the hair fibers are damaged, for instance as a result of their health status or of having been subjected to a conventional procedure deleterious to the hair, such as bleaching or coloring. Damaged hair fibers can display discontinuities in their outer surfaces, allowing for more water to penetrate the hair shafts as compared to healthy hair fibers. When the hair fibers are exposed to high temperatures (e.g., during styling with a hot iron, or drying with hot air), the residual water which can be present within the hair cortex may undergo explosive evaporation, further enlarging the defects of the damaged hair fibers or forming new micro-pores accelerating future permeation of water, the proliferation of such voids with each elevated heating significantly detracting from the hair integrity, possibly leading to hair breakage.

Without wishing to be bound by theory, it is believed that polymers formed with a relatively low cross-linking density behave in a thermoplastic manner, namely can reversibly become softer and malleable upon heating, while being sufficiently rigid upon cooling and at ambient temperatures to maintain a desired style to the treated hair. It is believed that this relative “flowability” of polymers having a relatively low cross-linking density allows them, upon heating of the hair fibers, to block or seal the pores or voids that may be present or have formed, especially in damaged hair. This “sealing effect” is expected to reduce water re-entry into the hair over time, thus decreasing the likelihood and/or extent of explosive evaporation of trapped water upon subsequent heating. Such reduction of water re-entry can be desirable for both damaged and undamaged hair, and hence, compositions forming polymers having a thermoplastic behavior by having a relatively low cross-linking density may be applied to both hair forms.

Hence, in some embodiments, when a polymeric network having a relatively low cross-linking density is desired, cross-linkers can be selected to have a relatively low number of cross-linking functions and/or be present in the composition at a relatively low concentration (or at a low ratio of cross-linkers to PBMs).

Other features readily appreciated by a skilled person may promote a relatively low or conversely a relatively high cross-linking density of a polymeric network formed by curable monomers as herein taught. For instance, relatively shorter cross-linkers (e.g., having a relatively low MW) may form polymers with denser cross-linking/tighter 3D networks, than relatively longer cross-linkers (e.g., having a relatively high MW) which may form looser networks. It is stressed that no single feature of a cross-linker could alone determine if the hair styling composition prepared therewith will tend to have, or not, a relatively low cross-linking density/thermoplastic behavior once polymerized. Still, a relatively low concentration, of a relatively long cross-linker having a relatively low amount of cross-linking functions can be expected to favor the formation of a cured polymer having a relatively lower cross-linking density than one prepared using a relatively high concentration, of a relatively short cross-linker having a relatively high amount of cross-linking functions.

As readily appreciated by a person skilled in the art of polymerization facilitated by cross-linkers, such compounds are typically present in an amount corresponding at least to a stochiometric reaction between the cross-linkable groups of the monomers and the corresponding reactive groups of the cross-linkers. Such minimal amount might already provide for an excess of cross-linkers, if some of the cross-linkable groups of the monomers and growing oligomers are hindered, in particular as curing proceeds towards the formation of more complex polymers. Nevertheless, in some embodiments, and in particular when cross-linkers may react with one another in addition to their ability to react with the monomers, it might be desired to include such curing facilitators in excess of their mere stochiometric concentration.

Suitable condensation-curable cross-linkers can be selected from reactive silanes having at least two silanol groups and a molecular weight of at most 1,000 g/mol, such as aminopropyltriethoxysilane (e.g., Dynasylan® AMEO), 3-isocyanatopropyltriethoxysilane, 3-aminopropyl(diethoxy)-methylsilane, methyltriethoxy-silane, or N-[3-(trimethoxysilyl)-propyl]ethylenediamine; mixtures of reactive silanes and amino-silanes (e.g., Evonik Dynasylan® SIVO 210); polybasic acids, such as succinic acid, adipic acid or citric acid; polyols, such as castor oil; polyamines, such as hexamethylenediamine or hexamethylene-tetramine (optionally combined with a dialkyl maleate, e.g., dimethyl maleate, diethyl maleate, or dibutyl maleate, their reaction product, potentially via Michael reaction, producing an active cross-linker that can react with the monomers of the present invention under the conditions taught herein); mono- and di-glycidyls, such as (3-glycidyloxypropyl)-trimethoxysilane or poly(ethylene glycol)diglycidyl ether; di-isocyanates, such as isophorone diisocyanate or 4,4′-methylenebis(cyclohexyl isocyanate); allylic compounds, such as allyl hexanoate or 1-methyl-4-(prop-1-en-2-yl)cyclohex-1-ene (limonene); and polyphenols, such as tannic acid. In particular embodiments, the condensation-curable cross-linker is aminopropyltriethoxysilane.

Multi-functional cross-linkers can be silsesquioxanes having organic glycidyl or methacrylate groups attached thereto. Such hybrid molecules contain an inner inorganic cage core, wherein organic groups are bound to said core. There may be as many as eight groups, increasing the cross-linking ability, and providing higher density to the cross-linked polymeric network. Such multi-functional cross-linkers include Glycidyl POSS® Cage Mixture EP0409 or Glycidyl Methacryl POSS® Cage Mixture MA0735, commercially available from Hybrid Plastics, USA. In a particular embodiment, such a hybrid cross-linker is used in the hair styling composition together with the cross-linker aminopropyltriethoxysilane.

Advantageously, but not necessarily, cross-linkers may additionally serve to modify the pH of the composition, facilitating the opening of the cuticle scales of hair fibers to which compositions including them are applied, and allow the PBMs, or part thereof, to penetrate the hair shaft.

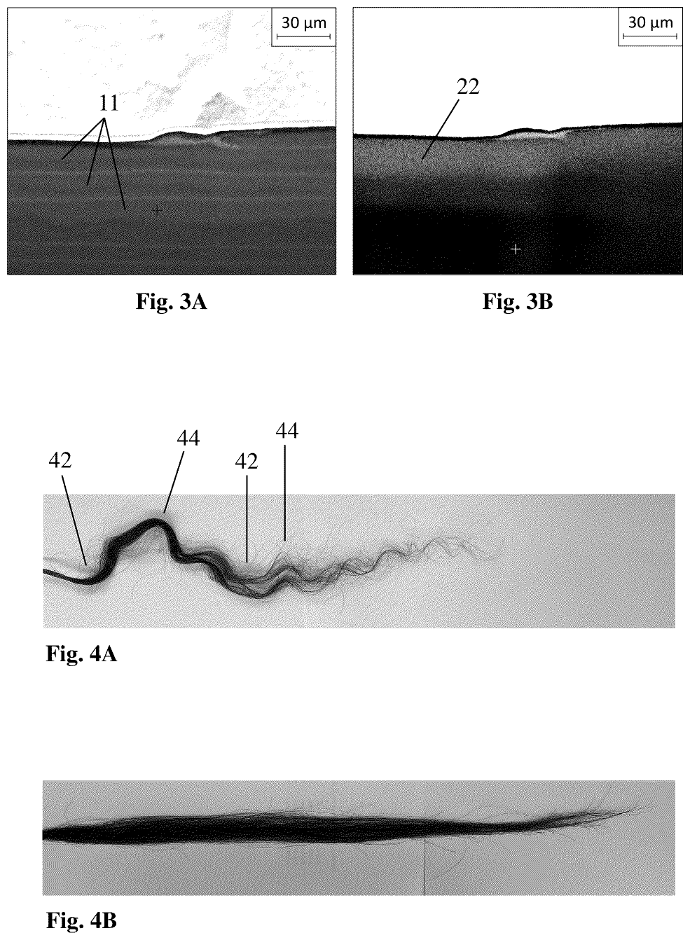

Without wishing to be bound by any particular theory, it is believed that the PBMs according to the present teachings are molecules sufficiently small (e.g., having a MW of 10,000 g/mol or less) to at least partially penetrate the fiber shaft where they may subsequently polymerize upon application of energy (e.g., thermal or electromagnetic, as suitable to induce polymerization of the monomers). Penetration of the PBMs into the hair fiber can be observed and monitored by microscopic methods, such as FIB-SEM (e.g., FIGS. 2B and 3B, to be addressed further below). When polymerization is effected while the hair fibers are in a desired modified shape, the resulting phenol-based oligomers (PBOs) and phenol-based polymers (PBPs), may maintain the fiber in the modified shape or delay the ability of the fiber to regain its native (un-modified) shape. Such steps shall be described in more details in following sections.

Reverting to the compositions that may be applied to individual fibers as a first step of the present hair styling method, when present, the cross-linkers, regardless of any effect they may additionally provide, may undergo at least partial hydrolysis, e.g., with water, prior to their combination with the PBMs. Alternatively, hydrolysis facilitators can be used to induce the hydrolysis following the combination of the cross-linkers with the PBMs. Suitable facilitators of such hydrolysis can be acids having (or providing to the composition) a pH between 4 and 6, such as salicylic acid and lactic acid, acetic acid, formic acid, citric acid, oxalic acid, uric acid, malic acid, tartaric acid, azelaic acid or propionic acid. The hydrolysis facilitators can be present in the composition being applied on the hair fibers and/or can be later spread thereon. Either way, partial hydrolysis of suitable cross-linkers is expected to enhance the activity of the cross-linkers, facilitating the condensation of PBMs leading to their polymerization. Hydrolysis facilitators can be viewed as one type of curing accelerators.

In some embodiments, the curing accelerators suitable for the hair styling compositions comprising PBMs, and methods of the present invention using the same, are suitable for condensation-polymerization, and can be selected from metal complexes (e.g., having as metal: Co, Mn, Ce, Fe, Al, Zn, Zr, Se or Cu), including for instance metal carboxylates such as acetyl acetonates or naphthenates; metal complexes with alkoxides, such as aluminum tri-sec-butoxide; metal soaps such as aluminium stearate and magnesium stearate; metal salen complexes such as N,N′-bis-(salicylidene)ethylenediamine complex with Fe or Mn; strong acids such as p-toluene-sulfonic acid, sulfuric acid, phosphoric acid or sulfosuccinic acid; and strong bases such as NaOH, KOH, NH₄OH.

It is noted in this context that while compounds have been for simplicity categorized according to their main role, such functions are not necessarily exclusive of others. For illustration, a curing accelerator (e.g., aluminum tri-sec-butoxide), typically used as a curing catalyst, may bear cross-linkable groups, such that the curing accelerator could also serve as a cross-linker, being incorporated into the formed polymeric network. In another example, dibutyl maleate, serving as an auxiliary polymerization agent, may also serve as a co-solvent, enhancing the miscibility of the PBM with the water in the aqueous phase.

In some embodiments, the PBMs of the present invention may further contain at least one addition-curable group, such as conjugated or non-conjugated double bonds, allowing the monomers to undergo both condensation-polymerization, occurring via the hydroxyl groups of the PBM, as well as addition-polymerization. For example, when the PBM is CNSL, its non-conjugated unsaturated alkyl side-chain at the R₄ position allows such polymerization by addition-curing under suitable conditions. Conditions suitable for addition-curing may include using within the composition a curing accelerator to open the double bond(s) of the side-chain, forming a radical, thus initiating the addition-polymerization. Alternatively, or additionally, the cross-linker may itself contain addition-polymerizable groups, the activation of which resulting in radical formation. The activated groups on the cross-linkers can react with activated groups on the PBM, or activated molecules of a same type may react one with another. Such addition-polymerizable groups that can be present in cross-linkers may be methacrylate groups (e.g., as existing on a silsesquioxane cage core, such as in the commercially available Glycidyl Methacryl POSS® Cage Mixture MA0735). Curing accelerators suitable for addition-polymerization include organic peroxides such as benzoyl peroxide, tert-butyl peroxy benzoate, di-tert-butyl peroxide, ortho- and para-methyl and 2,4-dichloro derivatives of dibenzoyl peroxide, dicumyl peroxide, alkyl peroxides (e.g., lauroyl peroxide, and 2-butanone peroxide), ketone peroxide and diacyl peroxide.

In some embodiments, when the PBMs and/or the cross-linkers contain at least one double bond (which renders the cross-linkers suitable for addition-curing with the PBMs), and especially at least two double bonds (e.g., short dienes), conjugated or non-conjugated, their exposure to atmospheric oxygen may induce an autoxidation reaction, resulting in a formation of the radical, allowing the polymerization or cross-linking to proceed by addition mechanism optionally in absence of a dedicated curing accelerator.

In some embodiments, cross-linkers suitable for addition-curing are straight, branched or cyclic alkene compounds including up to fifteen carbon atoms and containing a number of double bonds allowing for the formation of at least two radicals upon opening of the double bond(s). For instance, the alkene may contain at least two double bonds if positioned within the alkene chain (e.g., short fatty oils or short monoterpenes, such as myrcene (C₁₀H₁₆), geraniol, (C₁₀H₁₈O), carvone (C₁₀H₁₄O) and farnesene (C₁₅H₂₄)) or at least one double bond positioned at the terminus of the alkene chain. Short alkenes cross-linkers with double bonds at both terminus of the alkene chain (e.g., 1,5-hexadiene or 1,5-hexadiene-3,4-diol) are therefore also suitable.

Additional cross-linkers, having terminal double bonds at both ends of the chain include diallyl ethers (e.g., di(ethylene glycol), divinyl ether or 2,2-bis(allyloxymethyl)-1-butanol); diallyl sulfides; diallyl esters (e.g., diallyl adipate); acrylates (e.g., ethylene glycol diacrylate, ethylene glycol dimethacrylate, dipropylene glycol diacrylate, trimethylolpropane triacrylate and trimethylolpropane trimethacrylate); diallyl acetals (e.g., 3,9-divinyl-2,4,8,10-tetra-oxaspiro[5.5]undecane); triallyl cyanurate; and triallyl isocyanurate. Cross-linkers suitable for addition-curing of PBMs also include substituted or unsubstituted vinyl aromatic compounds (e.g., styrene or vinyl toluene); vinyl esters (e.g., vinyl acetate, vinyl benzoate, vinyl stearate or vinyl cinnamate); and vinyl alcohols (e.g., 10-undecen-1-ol). If a blend of cross-linkers is used, at least one of the cross-linkers needs be able to provide two radicals, another cross-linker optionally providing only one radical upon double bond opening.

Polymerization of the PBMs by addition-curing, either alone or in combination with additional ingredients of the present hair styling compositions, can be monitored by standard methods. By way of example, the iodine value of a composition (measured as gram of iodine per 100 grams of material) is expected to decrease as double bonds open to cross-link with other monomers or suitable ingredients. Hence, the formation of a synthetic polymer in an inner part of the hair fibers with any particular composition of the invention can be followed by determining the iodine value of the composition prior to its application and curing, as compared to the iodine value of a material extracted from the hair fibers following penetration and curing therein. Materials, including the synthetic inner polymer, can be extracted from hair fibers by diffusion (e.g., by dipping the hair samples in a suitable extracting liquid, such as water and/or IPA, at 40-70° C. for two to twelve hours) and concentrated to yield a sample adapted for the testing method. Iodine values can be determined by standard methods, such as described in ASTM D-1959.

While the compositions and methods according to the present teachings can be applied and implemented on hair fibers separated from a living subject (e.g., on a fur or on a wig), they are typically intended for application on hair of living mammalian subjects, in particular for use on human scalps. Therefore, while a number of cross-linkers, curing accelerators or other agents and additives as detailed hereinbelow may be used in compositions able to satisfactorily modify the shape of hair fibers, all such ingredients, as well as the PBMs, shall preferably be cosmetically acceptable. Ingredients, compositions or formulations made therefrom, are deemed “cosmetically acceptable” if suitable for use in contact with keratinous fibers, in particular human hair, without undue toxicity, instability, allergic response, and the like. Some ingredients may be “cosmetically acceptable” if present at relatively low concentration according to relevant regulations.

When the intended hair styling compositions are single-phase compositions, they are achieved when the PBMs are dissolved in a continuous aqueous phase containing at least one water-soluble hygroscopic agent and a suitable co-solvent. When the intended hair styling compositions are oil-in-water emulsions, they are achieved when the PBMs are emulsified and dispersed as oil droplets in a continuous aqueous phase, which also contains a water-soluble hygroscopic agent, and may optionally further include a suitable co-solvent. Curing facilitators, when present, should be miscible with the monomers while in the hair fibers, regardless of the phase from which they may be delivered to the hair cortex.

In some embodiments, the aqueous phase of the curable hair styling composition has a pH suitable a) to provide adequate charging to the hair fibers and the composition including inter alia the PBMs, b) to provide a suitable solubility of a compound in a medium (or on the contrary a lack thereof), and/or c) to provide suitable opening of the hair scales to facilitate penetration. While acidic pH (e.g., in a range of about 1-3.5) may also enable such effects, in some embodiments, the aqueous phase of the curable hair styling composition has an alkaline pH. Electing one non-neutral pH over another may depend on the chemical nature of the monomers and curing facilitators, some intrinsically contributing to an acidic or a basic pH, or being more potent at one pH over the other.

It is noted that, in some embodiments, the WHAs of the present compositions do not substantially interfere with the pH of the composition when added at low concentrations (e.g., 1 wt. % or less by weight of the composition). At such concentrations, the pH of the hair styling composition is typically similar in presence or absence of the WHA(s), such compounds increasing the pH or decreasing the pH by half-a-log or less. For illustration, if a hair styling composition has a pH of 7.5 without the WHA(s), the addition of up to 1 wt. % of the hygroscopic agent might adjust the pH to a value in a range of pH 7 to pH 8, a change in pH being in some embodiments of 0.4-log or less, 0.3-log or less, 0.2-log or less, or 0.1-log or less. Reverting to the illustration, a composition having a pH of 7.5 without WHA(s) may respectively have a pH in the range of 7.1-7.9, 7.2-7.8, 7.3-7.7, or 7.4-7.6, in presence of the WHA(s) at such low concentrations. This, however, is not essential, as some WHAs may favorably contribute to achieve a pH desired for the composition (e.g., adapted to promote hair scales opening and/or facilitate hair penetration) even at low concentrations, while others may have such a pH modulating effect at the concentration they are present.

While the pH of the hair styling compositions of the present invention can be adjusted to have any desired non-neutral pH to inter alia lift the hair scales to facilitate penetration of the monomers, such mechanism does not rule out the existence of additional ways of introducing monomers within the fiber cortex. For instance, the monomers and agents required for their polymerization or for the protection of the resulting polymer (or of its effect) may additionally be polar enough to diffuse through the hair scales, whether or not sufficiently opened for direct migration between the hair environment and its cortex.

Regardless of the form of the styling composition, and without being bound by theory, it is believed that an alkaline pH contributes inter alia to the opening of the cuticle scales by charging the surface of the hair fibers (due to chargeable groups, generally present on the fibers, e.g., carboxyl groups), thus allowing a better penetration inter alia of the monomers into the hair shaft. The alkaline pH may also contribute to the charging of the hair styling composition, increasing the zeta potential difference (Δξ) between the hair and the composition, a resulting higher gradient between the two facilitating the migration of the composition constituents towards the hair fibers for better contact.

In some embodiments, the hair styling composition (e.g., oil-in-water emulsion) has a pH of least 7, at least 8, at least 8.5, at least 9, at least 9.5, or at least 10. Typically, the pH of the composition does not exceed pH 11. In particular embodiments, the pH of the composition is between 7 and 9, between 7.5 and 9, between 8 and 10.5, between 9 and 10.5, or between 9.5 and 10.5.

Such an alkaline pH of a hair styling composition can be achieved by dispersing or dissolving the oil phase in which the PBMs reside with an aqueous phase at a suitable pH (e.g., to respectively form an emulsion or a single phase). The pH of the aqueous phase can be adjusted by using any suitable pH modifying agent at any concentration adapted to maintain the desired pH. Such agents include bases, such as ammonium hydroxide, sodium hydroxide, lithium hydroxide or potassium hydroxide. The pH modifying agents may also be amines, such as monoethanol-amine, diethanolamine, triethanolamine, dimethylethanolamine, diethyl-ethanolamine, morpholine, 2-amino-2-methyl-1-propanol, cocamide monoethanol-amine, aminomethyl propanol or oleyl amine. Alternatively, or additionally, other components of the hair styling composition, which are basic in nature, may provide or contribute to the alkaline pH of the composition (e.g., emulsion). For instance, the cross-linkers commercialized as Dynasylan® AMEO and Dynasylan® SIVO 210 are having such an effect in view of their amine groups.

Conversely, an acidic pH of 4.5 or less, 4 or less, or 3 or less may also contribute to the opening of the hair scales. Typically, the pH of a hair styling composition having such acidic pH is at least 1, at least 1.5, or at least 2, and generally between 1 and 4, between 1 and 3, between 1.5 and 3.5, between 2 and 4, or between 2.5 and 3.5. Such an acidic pH may be obtained using acids as pH modifying agents, which can be selected from acetic acid, perchloric acid, and sulfuric acid, to name a few. Alternatively, or additionally, other components of the hair styling composition, which are acidic in nature, may provide or contribute to the acidic pH of the composition (e.g., emulsion). For instance, the cross-linkers known as triethoxysilylpropylmaleamic acid and trihydroxysilylethylphenyl sulfonic acid are having such an effect in view of their respective acidic groups.

Since, as above exemplified, a number of compounds present in the composition may contribute to any of its particular property or function, whether dedicated for that purpose as primary role or inherently contributing to achieve it, the property sought for the composition is typically monitored at equilibrium. For illustration, if it is desired that a hair styling composition according to the present teachings has a pH, a polarity, a charge, or any other property of interest, within a particular range, such property can be arbitrarily determined 12 hours after having prepared the composition (even if a similar value could have been obtained upon completion of the composition preparation).

The aqueous phase, either in the oil-in-water emulsions or the continuous phase in which the PBMs are dissolved to produce the single-phase compositions, further includes at least one water-soluble hygroscopic agent (WHA). Such a hygroscopic agent, being dissolved within the aqueous phase, is believed to penetrate into the hair fibers together with the constituents of the hair styling composition enabling polymerization, and to settle in the gaps of the cross-linked PBMs network formed within the hair fibers. Upon elimination of water (e.g., during the application of thermal energy), the hygroscopic agent may crystallize, resulting in the formed polymeric network now containing crystals of the hygroscopic agent either intertwined therein or forming separate individual crystalline bodies. The crystallized state of the hygroscopic agent is believed to increase its water-absorption ability, thus enhancing its capacity to sequester and eliminate any water molecules penetrating the hair fibers (e.g., originating from environmental air humidity), and consequently, allowing for a long-lasting styling of the hair. Conversely, a crystallized WHA may serve as an internal reservoir of water molecules which may also desorb them under suitable conditions, for instance in a method of restyling or de-styling hair fibers previously treated and styled with the present compositions.

Hygroscopic agents suitable for the purpose of the present invention are soluble in water, i.e., having a solubility in pure deionized water at a pH of about 7.0 of more than 5 wt. %, and more typically, of more than 6 wt. %, more than 7 wt. %, more than 8 wt. %, more than 9 wt. %, or more than 10 wt. %, by weight of the water. In some embodiments, the WHAs are highly soluble with a solubility of 15 wt. % or more, 20 wt. % or more, or 30 wt. % or more, by weight of the deionized water. In some embodiments, the WHAs have similar solubilities with respect to the weight of the aqueous environment wherein they are to be disposed at the pH of the liquid, and for illustration, a WHA should not only have a solubility of more than 5 wt. % by weight of pure water, but also by weight of the hair styling composition (if a single aqueous phase) or in an aqueous phase of the composition (if an emulsion). Unless otherwise stated, all values refer to measurements made at room temperature under atmospheric pressure.

In some embodiments, the WHAs may be highly soluble in water, with a solubility of up to 150 wt. %, up to 125 wt. %, up to 100 wt. %, or up to 75 wt. %, by weight of water. In other words, up to 150 weight parts, up to 125 parts, up to 100 parts, or up to 75 parts of WHA would dissolve in 100 weight parts of water. It is stressed that the solubility of the WHA by weight of a liquid, is not to be assimilated with the concentration of the WHA in the liquid containing it. For illustration, assuming the WHA and the water are the sole constituents of a mix, then the concentration of a WHA having a solubility of up to 150 wt. % by weight of pure water could be of up to 60 wt. %, per weight of the aqueous mix, this relative concentration of the WHA further decreasing as other constituents (e.g., PBMs) are added to form a complete hair styling composition. The solubility might be the same or slightly lower in the aqueous phases containing all water miscible materials but the WHA, the WHAs having, in some embodiments, a solubility of up to 140 wt. %, up to 110 wt. %, up to 80 wt. %, or up to 50 wt. %, by weight of the composition or of an aqueous phase thereof.

While the hygroscopic agents used in the present compositions can be liquid at a room temperature, they are advantageously solid to further increase their residency within the hair fibers. Thus, in some embodiments, the at least one WHA has a melting temperature Tₘ higher than about 25° C. In particular embodiments, the Tₘ of the WHA is higher than the body temperature, or higher than external temperatures in extreme conditions, as it is undesirable that the WHA liquifies within the hair fibers that are in contact with the scalp, possibly leaching out of the fibers. Hence, the WHA may preferably have a Tₘ of 37° C. or more, 40° C. or more, 45° C. or more, or 50° C. or more. In some embodiments, the melting temperature Tₘ is lower than about 250° C., lower than about 200° C., lower than about 180° C., or lower than about 160° C.

Additionally, the WHA(s) can be selected to have a boiling temperature T_{b} greater than the boiling temperature of water, so that the WHAs would not readily evaporate as the hair fibers are treated to remove water (e.g., the hair being dried). In some embodiments, the at least one WHA has a T_{b} of 100° C. or more, 120° C. or more, or 140° C. or more. In some embodiments, the WHA has a T_{b} of 300° C. or less, 250° C. or less, or 225° C. or less. The temperatures characterizing a material (e.g., Tₘ, T_{b}, etc.) are typically provided by its manufacturer, but can be readily determined by standard methods, using for illustration Differential Thermal Analysis (DTA), Thermogravimetric Analysis (TGA) or Differential Scanning Calorimetry (DSC), such as described in ASTM E794-06, or ASTM 3418.

For similar reasons (e.g., increasing the likelihood of a prolonged residency of the WHAs within the hair fibers), the WHA(s) can be selected to have a vapor pressure lower than the vapor pressure of water (i.e., <2.3 kPa). In some embodiments, the WHA has a vapor pressure of 1.0 kPa or less, 0.5 kPa or less, 0.1 kPa or less, 10 Pascal (Pa) or less, or 1 Pa or less, as measured at 25° C. In some embodiments, the WHA has a vapor pressure of 1 milliPascal (mPa) or more, 10 mPa or more, or 50 mPa or more, as measured at 25° C. The vapor pressure of a material is typically provided by its manufacturer, but can be readily determined by standard methods, using for illustration DTA or DSC, as described for instance in ASTM E1194, ASTM D2879, or ASTM E1782.

To favor the hygroscopic agents in their competition with other compounds for the binding of water molecules, a suitable WHA preferably has a hydrogen bond energy (or hydrogen bonding energy) with water molecules that is greater than the hydrogen bond energy of water molecules among themselves (in pure water). In some embodiments, the at least one WHA has a hydrogen bond energy with water molecule of at least 21 kJ/mol, at least 22.5 kJ/mol, at least 25 kJ/mol, or at least 27.5 kJ/mol. In some embodiments, the at least one WHA has a hydrogen bond energy with water of at most 40 kJ/mol, at most 35 kJ/mol, or at most 32.5 kJ/mol. The hydrogen bonding energy of a material can be available from literature or estimated by known computer simulations according to empirical or semi-empirical approaches, for instance by the Density Functional Theory (DFT) calculations. Experimental studies indicating the formation of hydrogen bonds and the relative strength of bonding in various hydrogen-bonded complexes typically rely on crystallography and spectroscopy, such as infra-red (IR), nuclear magnetic resonance (NMR), microwave, electronic and Raman spectroscopy. For illustration, the hydrogen bond energy between an amine group (as can be found in a WHA) and water has been reported to be of about 29 kJ/mol, whereas the hydrogen bond energy of water molecules in pure water is of about 21 kJ/mol.

It is desirable that the hygroscopic agent does not substantially affect or modify the charge of the composition, hence, a WHA that is non-ionic or non-electrolyte is preferred. Yet, an ionic hygroscopic agent may be used, as long as present in an amount such that the zeta potential of the hair styling composition is sufficiently distinct from the zeta potential of the hair, so as to promote migration of the composition to the surface of the hair fibers and its retentions thereon. The effect of delta zeta potential on hair styling, a relatively higher absolute value being expected to enable a relatively greater penetration inter alia of the PBMs and WHAs, and a relatively prolonged styling effect is detailed further below.

It is further desired that the hygroscopic agent used in the present compositions (as any other material deemed suitable) has substantially no negative impact on the stability of the dispersion, such has no negative effect on the size of the emulsion droplets and/or on their size distribution, which may lead to a collapse of the emulsion (e.g., phase separation). The composition, if an emulsion, may have oil droplets not exceeding a few micrometers (e.g., having a D90≤20 μm and/or a D50≤10 μm, 5 μm, 2 μm, or 1 μm, parameters such as D10, D50, and D90 being measurable by Diffractive Light Scattering (DLS)).

In some embodiments, the water-soluble hygroscopic agent is selected from a group consisting of: amides (e.g., carboxamides, including aliphatic amides and amino acid amides); monosaccharides (e.g., glucose, fructose, galactose or mannose); disaccharides (e.g., sucrose or lactose); and combinations thereof.

In one embodiment, the WHA is a carboxamide having the general formula RC(═O)NR′R″, wherein R, R′, and R″ each independently represent an organic group or a hydrogen atom. In some embodiments, R can include a second carboxamide group. For illustration, methanamide (also referred to as formamide) and urea are carboxamides wherein R is respectively H or NH₂, and R′ and R″ are hydrogen atoms in both cases. R, R′, and R″ are typically relatively short molecular structures, for instance short linear, branched, or cyclic, substituted or unsubstituted, saturated alkyls having from 1 to 6 carbon atoms. Carboxamides wherein R is a short C₁-C₆ alkyl as aforesaid and R′═R″═H, include a) ethanamide (also referred to as acetamide) (R═CH₃), propaneamide (R═CH₂CH₃) and butanamide (R═CH₂CH₂CH₃), for illustration of short alkyls which can be branched if having a sufficient amount of carbon atoms; b) cyclopropane carboxamide, cyclobutane carboxamide, cyclopentane carboxamide and cyclohexane carboxamide, for illustration of short cyclic alkyls; and c) ethanediamide (also known as oxamide), propanediamide (also known as malonamide), butanediamide, pentanediamide and hexanediamide, for illustration of diamides. Carboxamides having, like urea, more than one amine group can be related to amino acid, and as such are often referred to as amino acid amides. This group of WHA compounds includes alanine amide, asparagine amide, glutamine amide, glycine amide, and proline amide. In a particular embodiment, the WHA is urea.

The single-phase compositions and the oil-in-water emulsions typically differ from one another by the relative amounts of water and co-solvents each may contain, thus each type will be separately discussed below. It should be noted that there might be overlap in the ranges of concentrations appropriate for each type of composition, as the relative amounts of water and co-solvents suitable for a particular type of composition also depends on the monomers, the curing facilitators, the auxiliary polymerization agents, the WHAs, or any other additive, as well as their respective amounts.

In some embodiments, the concentration of water in the single-phase composition is at least 2 wt. %, at least 5 wt. %, at least 10 wt. %, at least 15 wt. %, or at least 20 wt. % by weight of the single-phase composition. In some embodiments, the concentration of the water is at most 80 wt. %, at most 60 wt. %, at most 40 wt. %, at most 35 wt. %, or at most 30 wt. % by weight of the single-phase composition. In particular embodiments, the concentration of the water is between 2 and 80 wt. %, between 2 and 60 wt. %, between 2 and 20 wt. %, between 2 and 15 wt. %, between 10 and 40 wt. %, between 10 and 30 wt. %, or between 15 and 40 wt. % by weight of the single-phase composition.

In some embodiments, the concentration of water in the oil-in-water emulsion is, at least 40 wt. %, at least 45 wt. %, or at least 50 wt. % by weight of the oil-in-water emulsion. In some embodiments, the concentration of the water is at most 70 wt. %, at most 65 wt. %, or at most 60 wt. % by weight of the oil-in-water emulsion. In particular embodiments, the concentration of the water is between 40 and 70 wt. %, between 40 and 65 wt. %, or between 45 and 65 wt. % by weight of the oil-in-water emulsion.

In some embodiments, the concentration of the water-soluble hygroscopic agent in the oil-in-water emulsion is at least 10 wt. %, at least 12.5 wt. %, at least 15 wt. %, at least 17.5 wt. %, or at least 20 wt. %, by weight of the oil-in-water emulsion. In some embodiments, the concentration of the WHA in the oil-in-water emulsion is at most 50 wt. %, at most 48 wt. %, at most 46 wt. %, at most 44 wt. %, at most 42 wt. %, at most 40 wt. %, at most 38 wt. %, at most 36 wt. %, or at most 34 wt. % by weight of the oil-in-water emulsion. In other embodiments, the concentration of the WHA in the oil-in-water emulsion is between 10 wt. % and 50 wt. %, between 12.5 wt. % and 48 wt. %, between 15 wt. % and 46 wt. %, between 17 wt. % and 44 wt. %, or between 20 wt. % and 42 wt. %.

Water may not be the sole “liquid carrier” of the present compositions, and in some embodiments, the hair styling compositions can further contain at least one co-solvent. The at least one co-solvent can be selected from C₁-C₁₀ alcohols having at least one hydroxyl group, such as methanol, ethyl alcohol, isopropyl alcohol, 2-methyl-2-propanol, sec-butyl alcohol, t-butyl alcohol, propylene glycol, 1-pentanol, 1,2-pentanediol, 2-hexanediol, benzyl alcohol or dimethyl isosorbide; water-miscible ethers such as di(propylene glycol) methyl ether, diethylene glycol monoethyl ether, dioxane, dioxolane, or 1-methoxy-2-propanol; aprotic solvents such as ketones (e.g., methyl ethyl ketone, acetone), dimethyl sulfoxide, acetonitrile, n-methyl pyrrolidone, di-methyl carbonate or dimethylformamide; esters, such as C₁₂₋₁₅ alkyl benzoate; and mineral or vegetal oils, such as isoparaffinic fluids, olive oil, coconut oil or sunflower oil. In particular embodiments, the co-solvent is isopropyl alcohol. Without wishing to be bound by any particular theory, it is believed that an oily co-solvent (e.g., C₁₂₋₁₅ alkyl benzoate) may also contribute to the hydrophobicity of the final composition.

As readily appreciated by the skilled persons, some of these co-solvents can indifferently be mixed with the PBMs of the oil phase, with the aqueous phase, or in parts with both, during the preparation of an emulsion, where the phases are distinct, or during the preparation of a single phase, where the oil phase is dissolved in the aqueous-co-solvent phase. Therefore, when referring in the following to a combined concentration of the co-solvents, a number of situations are encompassed: a) a single co-solvent is used and mixed either with the PBMs or with the aqueous phase; b) a single co-solvent is used and mixed with both the PBMs and the aqueous phase; and c) two or more co-solvents are used mixed with at least one of the PBMs and the aqueous phase. Without wishing to be bound by any particular theory, co-solvents are believed to improve the surface tension of the oil phase so as to facilitate penetration inter alia of the PBMs, and/or to increase the miscibility cross-linkers, when present, within the PBMs, and/or to increase the miscibility of the PBMs within the aqueous phase to form a single-phase composition.

In some embodiments, the combined concentration of the co-solvents in the single-phase composition is at least 20 wt. %, at least 30 wt. %, at least 40 wt. %, or at least 50 wt. % by weight of the single-phase composition. The maximal amount of co-solvents may depend on the PBMs being selected, as well as on the presence of any additional ingredients. In any event, the concentration of co-solvents is such that the composition is in the form of a single-phase composition. In some embodiments, the combined concentration of the co-solvents is at most 80 wt. %, at most 75 wt. %, or at most 70 wt. % by weight of the single-phase composition. In particular embodiments, the combined concentration of the co-solvents is between 20 and 70 wt. %, between 30 and 70 wt. %, or between 35 and 65 wt. % by weight of the single-phase composition.

In some embodiments, the combined concentration of the co-solvents in the oil-in-water emulsion is at least 1 wt. %, at least 3 wt. %, at least 5 wt. %, or at least 7 wt. % by weight of the oil-in-water emulsion. The maximal amount of co-solvents may depend on the PBMs being selected, as well as on the presence of any additional ingredients. In any event, the concentration of co-solvents is such that the composition is in the form of an emulsion. In some embodiments, the combined concentration of the co-solvents is at most 20 wt. %, at most 18 wt. %, or at most 15 wt. % by weight of the oil-in-water emulsion. In particular embodiments, the combined concentration of the co-solvents is between 1 and 20 wt. %, between 5 and 18 wt. %, or between 7 and 15 wt. % by weight of the oil-in-water emulsion.

The single-phase compositions and oil-in-water emulsions can be prepared by any suitable method. For instance, the present compositions can be manufactured by mixing a first blend including the PBM(s), hence including a predominant portion of the oil phase, with a second liquid, including a predominant portion of the aqueous phase in which the WHA(s) would be dissolved. These distinct sub-compositions, respectively forming a “PBMs compartment” and an “aqueous compartment”, which include any desired additive, are each said to include a predominant portion of any of the two phases, as it cannot be ruled out that some of the compounds of an oil-in-water emulsion may actually partly migrate between the two phases. For instance, considering the polymerizable sub-composition, the PBMs may be insignificantly miscible in water and/or prepared in presence of a co-solvent (or any other component of the emulsion) exhibiting some miscibility with water, which upon mixing with the predominantly aqueous sub-composition may merge in part with the aqueous phase. When upon mixing of the two phases, one dissolves in the other, a single-phase composition is obtained instead of an emulsion.

If the oil-in-water emulsion is prepared by mixing a PBMs compartment with an aqueous compartment including the WHAs, each may comprise an amount of respective ingredient suitable to achieve desired concentration in the final oil-in-water emulsion, upon mixing of the two compartments in set ratios. By way of illustration, in some embodiments, the concentration of the combination of all PBMs (if more than one) in the PBMs compartment is at least 2 wt. %, at least 4 wt. %, or at least 6 wt. % by weight of the PBMs compartment. In some embodiments, the concentration of the PBMs is at most 40 wt. %, at most 35 wt. %, at most 30 wt. %, at most 25 wt. %, at most 20 wt. %, at most 15 wt. %, at most 13 wt. %, or at most 12 wt. % by weight of the PBMs compartment. In particular embodiments, the concentration of the PBMs is between 2 and 40 wt. %, between 2 and 35 wt. %, between 2 and 30 wt. %, between 2 and 25 wt. %, between 2 and 20 wt. %, between 2 and 15 wt. %, between 4 and 13 wt. %, or between 6 and 12 wt. % by weight of the PBMs compartment.

As single-phase compositions and oil-in-water emulsions according to the present teachings can be prepared by any additional suitable method, other than by dissolving or emulsifying a mixture of a PBMs compartment and of an aqueous compartment including the WHAs, the concentration of the PBMs is alternatively provided by weight of the total/final composition (e.g., the single phase or the emulsion).

In some embodiments, the combined concentration of the PBMs (if more than one) in the hair styling composition (e.g., oil-in-water emulsion) is at least 0.1 wt. %, at least 0.15 wt. %, at least 0.2 wt. %, or at least 0.25 wt. % by total weight of the composition. In some embodiments, the concentration of the PBMs is at most 5 wt. %, at most 3 wt. %, or at most 2 wt. % by weight of the hair styling composition. In particular embodiments, the concentration of the PBMs is between 0.1 and 5 wt. %, between 0.15 and 3 wt. %, or between 0.2 and 2 wt. % by weight of the hair styling composition.

In some embodiments, the PBM is maintained in an inert atmosphere, such as under argon or nitrogen, in order to reduce or eliminate any environmental factors (e.g., oxygen) that could induce premature and undesirable polymerization.

In some embodiments, the combined concentration of the cross-linkers present in the hair styling composition (if more than one) is at most 5 wt. %, at most 2.5 wt. %, or at most 2 wt. % by weight of the total composition (e.g., oil-in-water emulsion). In some embodiments, the combined concentration of the cross-linkers is at least 0.001 wt. %, at least 0.005 wt. %, at least 0.01 wt. %, at least 0.05 wt. %, at least 0.1 wt. %, or at least 0.2 wt. % by weight of the total composition. In particular embodiments, the cross-linkers are present at a combined concentration between 0.001 and 5 wt. %, between 0.005 and 5 wt. %, between 0.01 and 5 wt. %, between 0.05 and 5 wt. %, between 0.01 and 2.5 wt. %, between 0.05 and 2 wt. %, between 0.1 and 2.5 wt. %, or between 0.2 and 2 wt. % by weight of the total composition. When considering the weight per weight ratio between the PBM(s) and their cross-linkers, this ratio can be between 1:15 and 10:1, between 1:15 and 7.5:1, between 1:15 and 5:1, between 1:10 and 2.5:1, between, or 1:5 and 5:1.

When compositions for forming a polymeric network having a relatively low cross-linking density are desired, so as to obtain a polymeric skeleton having a thermoplastic behavior, a relatively low concentration of cross-linkers can be utilized. In such cases, the cross-linkers can be present at a combined concentration between 0.001 wt. % and 0.5 wt. %, between 0.05 wt. % and 0.3 wt. %, or between 0.07 wt. % and 0.2 wt. % by weight of the total composition. When considering the weight per weight ratio between the PBM(s) and their cross-linkers, the ratios adapted for a relatively low cross-linking density can be between 10:1 and 2.5:1, or between 7.5:1 and 2.5:1.

If the curing process involves thermal energy, the cross-linkers are preferably selected to provide curing at a temperature elevated relatively to ambient temperature and/or at a rate sufficiently slow at room temperature, to prevent or reduce spontaneous curing during storage and/or application of the hair styling composition. To be feasible for use on living subjects, the curing temperature of a suitable cross-linker need not be too high (e.g., the hair fibers being between 50° C. and 60° C.), and both the curing temperature and curing rate of the cross-linkers can be selected to provide curing under reasonable conditions.

In some embodiments, the combined concentration of the curing accelerators (if more than one) is of at most 50 wt. %, at most 45 wt. %, or at most 40 wt. %, at most 35 wt. %, at most 30 wt. %, at most 25 wt. %, at most 20 wt. %, at most 15 wt. %, at most 10 wt. % or at most 5 wt. % by weight of the or PBM(s), the curing accelerators optionally being present at least 0.01 wt. % of the PBM(s). When considering the amount of the curing accelerators by weight of the total hair styling composition (e.g., oil-in-water emulsion), they are generally present in very low concentrations. In some embodiments, the combined concentration of the curing accelerators is of at most 5 wt. %, at most 3 wt. % or at most 2 wt. % by weight of the hair styling composition, the curing accelerators optionally being present at least 0.001 wt. % of the hair styling composition.

When peroxides are used as curing accelerators for addition-polymerization, their amount should be carefully considered in view of their ability to bleach the hair. Therefore, the amount of peroxides should be high enough to activate the polymerization, and low enough to avoid significantly bleaching the hair.

In some embodiments, the concentration of the curing facilitators (i.e., the combined concentration of cross-linkers and curing accelerators, whether used for addition or condensation polymerization) present in the hair styling composition is between 0.001 wt. % and 15 wt. %, between 0.001 wt. % and 10 wt. %, between 0.001 wt. % and 5 wt. %, between 0.05 wt. % and 15 wt. %, between 0.1 wt. % and 10 wt. %, or between 0.5 wt. % and 5 wt. % of the total hair styling composition.

In some embodiments, the single-phase composition or the oil-in-water emulsion may further contain at least one additive, adapted to enhance one or more properties of the hair styling composition. The additive can, for instance, be an auxiliary polymerization agent, an emulsifier, a wetting agent, a thickening agent, a charge modifying agent, or any other such ingredients traditionally found in hair styling compositions (e.g., fragrances).

In some embodiments, an auxiliary polymerization agent may be added to enhance and facilitate the polymer formation. Such auxiliary polymeric agents bear at least one functional group which, together with the polymerizable groups of the PBM or with the functional group(s) of the cross-linker or of suitable curing accelerator, increases the concentration of any functional groups that are available for cross-linking. A higher concentration of the functional groups contained within the auxiliary polymerization agent is believed to contribute to a higher degree of cross-linking facilitation. In view of the presence of at least one functional group, auxiliary polymerization agents may bind to the growing polymeric network. Preferably, the density of functional groups in the auxiliary polymerization agent should be high enough to allow using auxiliary polymerization agent having a molecular weight below 10,000 g/mol, 5,000 g/mol, or 3,000 g/mol, such a size not hampering its ability to penetrate into the hair shaft.

The functional groups contained within the auxiliary polymerization agent may be hydroxyl groups (—OH), carboxyl groups (—COOH), amine groups (—NH₂), or carbonyl groups (C═O). Suitable auxiliary polymerization agents may also bear functional groups such as anhydrides, isocyanates and isothiocyanates, which are capable of reaction with e.g., amine cross-linkers. Other suitable auxiliary polymerization agents may bear groups that can be further functionalized by other reactants present in the composition, such as double bonds, which can be opened (e.g., by an amine cross-linker, or alternatively in a Michael addition reaction or even a PBM “activated” to contain reactive radicals).

Exemplary auxiliary polymerization agents can be selected from: shellac, rosin gum, alkyl- or aryl-substituted maleates (e.g., dimethyl maleate and dibutyl maleate), oily diesters such as sebacates (e.g., bis(2-ethylhexyl) sebacate), fatty oils having alkene chains of sixteen carbon atoms or more, including terpenes and terpenoids (e.g., squalene and lycopene), fatty amines, (e.g., oleyl amine) and non-conjugated unsaturated fatty acids, such as arachidonic acid, linoleic acid and linolenic acids, conjugated fatty acids, such as retinoic acid, eleostearic acid, licanic acid and punicic acid, and triglycerides of such fatty acids containing conjugated or non-conjugated double bonds, such as pomegranate seed oil, chia seed oil, perilla seed oil, raspberry seed oil and kiwi seed oil. Alkenes that may serve as auxiliary polymerization agents are distinguished from alkenes that may serve as cross-linkers, by having a higher number of carbon atoms (e.g., 13 or more) and possibly a higher number of double bonds per molecule (e.g., 3 or more). Furthermore, auxiliary polymerization agents having unsaturated alkene chains can be characterized by an iodine number of 100 g iodine or more per 100 g agent, such value typically not exceeding 400.

Auxiliary polymerization agents having a thermoplastic behavior, such as shellac, may assist in the formation of a polymeric network having a thermoplastic behavior, whereas auxiliary polymerization agents lacking a thermoplastic behavior, such as fatty oils, may assist in the formation of a polymeric network having a relatively higher cross-linking density.

In some embodiments, the auxiliary polymerization agents used for the purposes of the present invention are hydrophobic, which, beyond their cross-linking enhancement within the hair fiber, might also assist in protecting the hair against moisture penetration.

In a particular embodiment, the auxiliary polymerization agent is shellac, a natural bioadhesive resin, collected from the secretion of an insect, which has a number of synthetic chemical equivalents. Generally, purified wax free shellacs have an average molecular weight between about 600 and 1,000 g/mol, and though there are controversies about their true structure, being a mixture of various components, they are known to contain repeating units of hydroxyl and carboxyl functional groups, together with olefinic and aldehyde function. Shellacs may be supplied with variable acid number of up to 150 mg KOH/g, the acid number being typically in the range of 65-90 mg KOH/g, and hydroxyl values generally between 180 and 420 mg KOH/g. The acid number of shellac is typically provided by its manufacturer, and can also be determined by conventional methods, such as an acid-base titration, wherein a known amount of shellac is titrated with potassium hydroxide (KOH) base, for instance, according to procedures described in ASTM D664, the acid number being expressed as milligram of KOH per gram of shellac.

In some embodiments, the combined concentration of the auxiliary polymerization agents (if more than one) is between 0.01 wt. % and 2 wt. %, between 0.05 wt. % and 2 wt. %, between 0.1 wt. % and 1.7 wt. %, or between 0.1 and 1.5 wt. % by weight of the hair styling composition.

The hair styling composition, if an oil-in-water emulsion, may further contain an emulsifier, so as to facilitate the formation of the emulsion and/or to prolong its stability. In some embodiments, the emulsifier is a non-ionic emulsifier, preferably having a hydrophile-lipophile balance (HLB) value between 2 to 20, between 7 to 18, between 10 to 18, between 12 to 18, between 12 to 17, between 12 to 16, between 12 to 15, or between 13 to 16 on a Griffin scale. Suitable emulsifiers can be water-soluble (e.g., having an HLB value between 8 and 20), such as polysorbates (often commercialized as Tweens), ester derivatives of sorbitan (often commercialized as Spans), acrylic copolymers (e.g., commercially available as Synthalen® W2000), and combinations thereof, or oil-soluble, such as lecithin and oleic acid (e.g., having an HLB value between 2 and 8). It is to be noted, that some constituents of the hair styling compositions selected for other functions may also serve as emulsifiers. Such an example is linoleic acid, generally used as an auxiliary polymerization agent, which may also serve as an emulsifier due to its polar head and fatty chain.

In order to facilitate a penetration of the PBMs into the hair fibers, the composition should be able to properly spread over the fibers to permit adequate contact. Adequate coating of the fibers by the composition during its application is expected to favor penetration, believed to be by capillary effect, of the monomers into the hair to form the synthetic polymer able to constrain the desired shape. Proper wetting of a surface can theoretically be improved by tuning the surface tension of the hair styling composition measured in milliNewton per meter (mN/m) to be lower than the surface energy of the fibers. Such properties can be determined by standard methods, and for instance according to procedures described in ASTM D1331-14, Method C.

Virgin hair fibers, which have not been previously treated by any kind of hair modifying treatment, typically have a surface energy of about 25-28 mN/m, whereas damaged hairs generally have a higher surface energy, chemically bleached hair fibers, for instance, being in the range of 31-47 mN/m. Among the many differences between damaged and undamaged hairs, the increased presence of naturally occurring fatty acids on undamaged hairs is believed to contribute to their relatively lower surface energy. In view of the above ranges, it can be assumed that when working with a composition having a surface tension of less than 25 mN/m, suitable wetting would be observed on all hair types. It was surprisingly found that hair styling compositions having a surface tension that is too low, do not provide the expected results as far as monomer penetration is concerned. The Inventors have discovered that, counterintuitively, compositions having a surface tension relatively higher than deemed theoretically appropriate are more suitable for the purpose of the present invention. Without wishing to be bound by theory, the absence of fatty acids within the hair shaft is believed to increase the surface energy perceived within the hair to be sufficiently higher than that measurable on the outer surface of the hair to require selection of a particular range of surface tensions for compositions intended to penetrate the hair shaft.

In some embodiments, the compositions of the present invention have a surface tension between 25 and 60 mN/m, between 25 and 55 mN/m, between 25 and 50 mN/m,

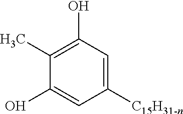

between 25 and 45 mN/m, or between 25 and 40 mN/m. between 25 and 35 mN/m, or between 30 and 40 mN/m.

The compositions of the present invention which are suitable for virgin hair, are also appropriate for previously treated hair fibers. However, in some embodiments, the styling compositions may display a surface tension adapted to sufficiently coat damaged hairs, while not being satisfactory enough for virgin hair fibers.

Wetting agents can be added to the composition, at any suitable concentration allowing to decrease its surface tension to be within any of the afore-described suitable ranges. Exemplary wetting agents can be silicone-based, fluorine-based, carbon-based or amine-alcohols. Silicone-based wetting agents can be silicone acrylates (such as SIU 100 by Miwon Specialty Chemical). Fluorinated wetting agents can be perfluorosulfonic acids (such as perfluorooctanesulfonic acid) or perfluorocarboxylic acids (such as the perfluorooctanoic acid). Carbon-based wetting agents can be ethoxylated amines and/or fatty acid amide (e.g., cocamide diethanolamine), fatty alcohol ethoxylates (e.g., octaethylene glycol monododecyl ether), fatty acid esters of sorbitol (e.g., sorbitan monolaurate), polysorbates and alkyl polyglucosides (e.g., lauryl glucoside). Amine-functionalized silicones can also be used as wetting agents (such as amo-dimethicone or bis-aminopropyl dimethicone), as well as alkanolamines (such as 2-amino-1-butanol and 2-amino-2-methyl-1-propanol). Wetting agents, if added, are typically present in the hair styling composition (e.g., oil-in-water emulsion) at a concentration of at least 0.001 wt. %, at least 0.01 wt. % or at least 0.1 wt. %; at most 1.5 wt. %, at most 1.4 wt. % or at most 1.3 wt. %; and optionally between 0.001 and 1.5 wt. %, between 0.01 and 1.4 wt. % or between 0.1 and 1.3 wt. % by weight of the composition.

Alternatively, or additionally, some of the components of the hair styling composition present therein to serve a different function may contribute to the surface tension of the hair styling composition. For instance, the cross-linker aminopropyltriethoxysilane (e.g., Dynasylan® AMEO) may reduce the surface tension of the composition, whereas linoleic acid which can be used as an auxiliary polymerization agent and as an emulsifier may increase it. The surface tension of the hair styling composition may accordingly be adjusted by selecting suitable concentration(s) of such components. Co-solvents may also contribute to the wetting ability of the composition towards hair fibers, in addition to contributing by their chemical formula and relative concentration to the type of hair styling composition that may be formed.

In some embodiments, a thickening agent can be added to provide a desired viscosity, generally to the aqueous phase of the oil-in-water emulsion or aqueous compartment. The viscosity should be sufficiently low to allow easy application of the composition to the hair so as to satisfactorily coat all individual fibers, but high enough to remain on the hair fibers for sufficient time and prevent dripping. A relatively low viscosity may also facilitate penetration of the PBMs into the hairs by diffusion and/or capillarity. Exemplary thickening agents can be hyaluronic acid, poly(acrylamide-co-diallyl-dimethyl-ammonium chloride) copolymer (Poly-quaternium 7, e.g., by Dow Chemicals), quaternized hydroxyethyl cellulose (Poly-quaternium 10, e.g., by Dow Chemicals), hydroxypropyl methylcellulose, etc. Thickening agents, if added, are typically at a concentration of at least 0.1 wt. %; at most 10 wt. %; and optionally between 0.5 wt. % and 5 wt. % by weight of the aqueous phase or single-phase.

In order to facilitate the migration and/or retention of the PBMs to the surface of the hair fibers, which in turn may increase their permeation therein, there should preferably be a difference between the zeta potential of the composition and the hair. For example, the zeta potential of the hair styling composition at its pH (or ξ_{c}) should preferably be more negative or more positive than a zeta potential of the mammalian hair fibers (or (h) at the same pH. In some cases, the ingredients used in the composition may provide, in addition to any other function, sufficient charging of the composition to achieve such a gradient of zeta potential values. For instance, pH modifying agents, wetting agents and/or amine-based cross-linkers may contribute to suitable charging of the oil-in water emulsion. In some embodiments, an agent dedicated to this effect, referred to as a charge modifying agent, can be added to the composition. For illustration, a water-insoluble, non-reactive amino-silicone oils may be added to the oil phase of the emulsion to modulate its zeta potential.

In some embodiments, the difference between the zeta potential of the composition ξ_{c} and the zeta potential of the hair fibers (h to be treated thereby, also termed the zeta differential or delta zeta potential (Δξ_{c-h}) is in absolute terms at least 5 mV, at least 10 mV, at least 15 mV, at least 20 mV, at least 25 mV, at least 30 mV, or at least 40 mV. In some embodiments, Δξ_{c-h} absolute value is within a range of 5 to 80 mV, 10 to 80 mV, 10 to 70 mV, 10 to 60 mV, 15 to 80 mV, 15 to 70 mV, 15 to 60 mV, 20 to 80 mV, 20 to 70 mV, 20 to 60 mV, 25 to 80 mV, 25 to 70 mV, 25 to 60 mV, 30 to 80 mV, 30 to 70 mV, 30 to 60 mV, 35 to 80 mV, 35 to 70 mV, or 35 to 60 mV. Such values are preferable to set an initial charge gradient driving inter alia the PBM(s) (e.g., as droplets) towards the hair fibers for their penetration therein together with the WHA(s). Understandingly, such gradient decreases over time, as the materials of the compositions initially accumulates on the hair outer surface modifying its zeta potential. This process is self-terminating, the migration from the composition to the hair ceasing once the gradient becomes too low (e.g., when the delta zeta potential becomes lower than 5 mV). Zeta potential can be determined by standard methods using any equipment suitable for the measurement of charge of dispersed particles.

The composition may also comprise any other additive customary to cosmetic compositions, such as preservatives, antioxidants, bactericides, fungicides, chelating agents, vitamins and fragrances, or customary to hair styling compositions, such as hair detangling agents and hair conditioning agents, the nature and concentration of which need not be further detailed herein.

The composition may also comprise any other additive customary to the form in which the hair styling composition is to be applied, such as propellants if the composition is to be sprayed, the nature and concentration of which need not be further detailed herein.

The mixing and/or emulsification of the aforesaid materials can be performed by any method known in the art. While manual shaking may suffice, various equipment, such as a vortex, an overhead stirrer, a magnetic stirrer, an ultrasonic disperser, a high shear homogenizer, a sonicator and a planetary centrifugal mill, to name a few, can be used, typically providing more uniform compositions, for instance more homogenous populations of oil droplets in the aqueous phase of an oil-in-water emulsion.

In some embodiments, the hair styling composition can be prepared by mixing or emulsifying the contents of a PBM compartment and an aqueous compartment including the WHAs, this combination being performed soon after each of the respective parts are ready. However, in alternative embodiments, the mixing of the two compartments can be deferred. In particular when the composition comprises PBM(s) and at least one curing facilitator (e.g., a cross-linker) prone to separate into distinct phases in a complete final composition, it may be desired to allow pre-polymerization of such materials in a same polymerizable compartment. In some embodiments, the pre-polymerization step is performed on a sole mixture of PBM(s) and curing facilitators, and not on the entire contents of a PBM compartment if due to include additional materials that may adversely affect the process or simply delay it. In other embodiments, pre-polymerization is performed on the PBM(s) alone, prior to their combination with the curing facilitators or any other component of the PBM compartment. Such pre-polymerization can be referred to as “self pre-polymerization”. Without wishing to be bound by theory, when the PBM(s) contain an unsaturated side-chain, such as CNSL, such self pre-polymerization is believed to occur by the opening of the double bond(s) under suitable conditions (e.g., elevated temperatures), forming radicals that are available for polymerization with other CNSL molecules, via addition-polymerization.

Such pre-polymerization, if needed and whether or not in presence of curing facilitators, should have a long enough duration to prevent the separation of the monomers and the curing facilitators into distinct phases upon mixing with additives of the PBM compartment and/or with the contents of an aqueous compartment to an extent significantly delaying polymerization within the hair fibers following application of the mixed composition. But the pre-polymerization should be short enough so that the oligomers that may form in this process (whether of cross-linkers or monomers by themselves or of cross-linkers and monomers ones with the others) remain sufficiently small to penetrate within the hair fibers following application of the composition. It is believed that the pre-polymerization results in the formation of oligomers (regardless of composition) at the expense of the relevant building blocks (e.g., monomers and/or cross-linkers) present in the pre-polymerized compartment. This process can be monitored by a viscosity of the pre-polymerized mixture of monomers and curing facilitators increasing with time.

In some embodiments, the viscosity of the pre-polymerized PBM compartment (optionally containing the at least one curing facilitator and/or auxiliary polymerization agent) is between 50 and 1,000 millipascal-second (mPa·s), between 70 and 800 mPa·s, between 100 and 600 mPa·s, between 200 and 400 mPa·s, or between 300 and 400 mPa·s, as measured at 25° C. and a shear rate of about 200 sec⁻¹.

The pre-polymerization step can be performed at ambient conditions, such as at room temperature, but it can be further accelerated by any mean adapted to induce and/or enhance polymerization, for instance by heating of the mixture. The pre-polymerization step can be performed in an inert atmosphere, such as under argon or nitrogen, in order to reduce or eliminate any environmental factors that could interfere with the pre-polymerization reaction (e.g., oxygen). The conditions for pre-polymerization, if performed, can depend on the type of PBM, as well as on the selected cross-linker, and on the relative proportions of all participating compounds.

In some embodiments, pre-polymerization can be performed at a temperature of at least 20° C., at least 30° C., at least 40° C., at least 60° C., or at least 100° C. In other embodiments, the pre-polymerization, when performed, is executed at a temperature of at most 200° C., at most 180° C., at most 170° C., or at most 150° C. In further embodiments, the pre-polymerization, can be performed at a temperature between 20° C. and 200° C., between 50° C. and 200° C., between 20° C. and 150° C., between 30° C. and 150° C., between 40° C. and 150° C., between 80° C. and 150° C., between 50° C. and 140° C., or between 110° C. and 150° C. The pre-polymerization may be performed at relatively lower temperatures in the afore-said range, such as between 20° C. and 60° C., between 25° C. and 60° C., between 30° C. and 60° C., or between 40° C. and 60° C., or at relatively higher temperatures, such as between 100° C. and 150° C. or between 150° C. and 200° C.

The duration of pre-polymerization may depend upon the temperature at which such a step is performed, as well as on the nature of the compounds participating in the reaction and their relative proportions. In some embodiments, the pre-polymerization is performed for a duration of at least 5 minutes, at least 10 minutes, at least 20 minutes, at least 30 minutes, at least 40 minutes, at least 50 minutes, at least 60 minutes, at least 120 minutes or at least 180 minutes. Typically, the duration of pre-polymerization does not exceed 24 hours, 18 hours or 12 hours, when performed at relatively mild temperature, but can be shortened if performed at relatively higher temperatures (e.g., between 150° C. and 200° C.) which may require less than 8 hours, less than 5 hours or less than 4 hours. In some embodiments, the pre-polymerization is performed for a duration between 5 and 60 minutes, between 10 and 50 minutes, or between 15 and 40 minutes and at a temperature of between 50° C. and 200° C., between 80° C. and 150° C., or between 110° C. and 150° C.

Following pre-polymerization, additives can optionally be added to the pre-polymerized compartment, and/or an aqueous compartment can be combined therewith to form the hair styling composition.

The hair styling composition (e.g., oil-in-water emulsion) can be readily applied following its preparation or within a time period during which it remains suitably stable and potent. For instance, if an emulsion, the composition can be applied as long as the oil droplets are within their desired size range (e.g., of no more than a few micrometers, typically less than 10 μm), provided that the PBMs have not fully polymerized in vitro. More generally, the hair styling composition can be used as long as a sufficient amount of PBMs is available to at least partially penetrate the hair fiber, so as to polymerize therein. In some embodiments, the single-phase composition or the emulsion is applied to the hair fibers within at most 30 minutes from its dissolution or emulsification, or within at most 20 minutes, at most 10 minutes, or at most 5 minutes.

In some embodiments, prior to applying the hair styling composition, either as a single-phase composition or as an oil-in-water emulsion, the hair fibers may be pre-treated.

A common pre-treatment that may be performed prior to applying the hair styling composition is a cleaning pre-treatment, wherein any residual materials that may be present on the hair, such as hair products, dirt or grease, are removed to clean the hair fibers. This can be done by applying any suitable cleaning products, such as sodium lauryl sulfate, this washing being followed by the rinsing of the hair fibers with excess water.

Another pre-treatment, which may follow cleaning or be performed independently, is a drying pre-treatment, wherein rinsing water or residual moisture can be removed from the hair. This removal of water molecules from the hair fibers, typically achieved by heating of the hair, is believed to break hydrogen bonds that may have formed either on the cuticle scales' surface and/or within the hair shaft.

As used herein in the specification, unless clear from context or otherwise stated, the term “residual moisture”, with regards to the hair fibers, refers to water that is present either on the outer surface of the cuticle scales, between and/or below the scales (i.e., in the cortex or medulla), originating from the hair being exposed to humidity (e.g., to ambient humidity or as a result of hair wetting). Understandably, complete removal of residual moisture is very difficult to realize, as the hair is always exposed to ambient humidity which is rarely null. Nevertheless, low levels of residual moisture are achievable, or can be temporarily achieved by applying energy, mainly thermal (i.e., heat), to the hair. Heat sufficient to achieve minor levels of residual moisture can be applied to the hair by any conventional method, e.g., using a hair dryer or a flat or curling iron for enough time. Regardless of the method employed to reduce the amount of water molecules in the hair, such a step can alternatively be referred to as a drying treatment or step.

When considering hair having at least a wavy appearance, one can readily visually assess that enough hydrogen bonds are broken by a drying pre-treatment, as sufficient drying results in a transient relaxation of the waves, the hair fibers being eventually completely flattened at the end of such a step, if so desired. Alternatively, as is the case for straight hair, the duration of a drying pre-treatment can be arbitrarily set as a function of the drying device being used and the temperature it may apply to the hair fibers. For instance, flat or curling irons which may directly apply by heat conduction temperatures of about 200° C. to the hair can achieve sufficient breakage of hydrogen bonds within a few minutes, whereas conventional hair dryers which depending on the distance from the hair they are used may apply relatively lower temperatures by heat convection, could require relatively longer drying duration. Typically, drying the hair fibers can be performed by heating areas of the hair fibers up to a temperature of at least 40° C., at least 50° C., at least 70° C., at least 80° C., or at least 100° C. for no more than 5 seconds at a time, such drying treatment taking up to 5 minutes for hair swatches when the heating proceeds from one end of the swatch to the other.

In some embodiments, the residual moisture level following such a drying treatment (if performed) and/or prior to application of the present compositions is at most 5 wt. %, at most 4 wt. %, at most 3 wt. %, at most 2 wt. % or at most 1 wt. % by weight of the hair fibers. Such amount can be determined by standard methods, using, for instance, thermogravimetric analysis, or near infrared technologies, such as opto-thermal transient emission radiometry.

Alternatively, or additionally, the heating that may inter alia contribute to the cleavage of hydrogen bonds within the keratin polymer and/or within the materials of the hair styling composition having penetrated the hair fibers, is the one a) optionally applied during the application of the composition (e.g., the composition being heated prior to its application); b) optionally applied during the incubation of the composition on the hair fibers; and/or c) applied during the styling of the hair fibers following the application of the composition. Regardless of its effect on hydrogen bonds, if any, the heating promotes the diffusion rate of the monomers/oligomers and/or the curing of the polymer within the hair fibers.

A third possible pre-treatment, which may follow cleaning and/or drying, or be performed independently, involves the application of a pre-treating composition intended to remain on the hair fibers during the performance of the hair styling method. The hair pre-treating composition can protect the hair fibers during the application of the hair styling composition, in particular during the application of heat, it can facilitate the performance of steps of the present methods, and/or it may enhance the properties of the hair styling compositions.

The hair pre-treating compositions should not undermine the effects sought by the present compositions and methods, and for instance should not interfere with the cuticles opening, with the migration of the styling composition towards the hair surface, with the penetration of the styling composition into the hair shaft, with the polymerization of the PBM(s) or sought activity of the WHA(s), and any like effect.

Typically, the hair pre-treating composition consists of an oil which can be applied to the hair fibers so as to form an ultra-thin oily layer on the surface of the fibers prior to their treatment with the hair styling composition.

In some embodiments, the oil used for this pre-treatment step or hair pre-treating composition has a solubility in water of 5 wt. % or less, 4 wt. % or less, 3 wt. % or less, 2 wt. % or less, or 1 wt. % or less, by weight of the water, as measured at a temperature of 25° C.

Factors rendering a hair pre-treating composition suitable for the present methods share similarities with some of the properties already described for the sake of the hair styling compositions and will only be mentioned briefly.

First, the hair pre-treating composition, which can be referred to as a pre-treating oil, should properly wet the hair fibers. For that purpose, the pre-treating composition or the oil therein should have a surface tension that is lower than the hair fiber surface energy. In some embodiments, the pre-treating composition or the oil have a surface of 35 mN/m or less, 30 mN/m or less, or 25 mN/m or less.

Secondly, in order to remain on the hair fibers and not evaporate during the application of energy, if so desired, the hair pre-treating composition or the oil therein should be essentially non-volatile during the process. Accordingly, in some embodiments, the pre-treatment oil has a vapor pressure of less than 40 Pa, less than 35 Pa, or less than 30 Pa. In other embodiments, the oil has a vapor pressure of more than 0.1 Pa, more than 0.2 Pa, or more than 0.5 Pa. In some embodiments, the pre-treatment oil has a vapor pressure between 0.1 Pa and 40 Pa, between 0.2 Pa and 35 Pa, or between 0.5 Pa and 30 Pa. The oil vapor pressure is measured at a temperature of 25° C.

In order to facilitate the coating of the hair fibers with a desired hair pre-treating composition, electrostatic attraction between the two can be promoted, the hair pre-treating composition (e.g., the oil pre-treatment) preferably having a zeta potential (ξₒ) that is sufficiently different from the hair fibers zeta potential (ξh). The (o of the pre-treating composition should also be sufficiently different from the zeta potential of the styling composition (ξ_{c}), in order to allow attraction inter alia of the PBMs to the oil pre-treatment layer formed on the hair fibers. Accordingly, in some embodiments, the delta zeta potential between the pre-treating composition and the hair (Δξₒ₋ₕ) and the delta zeta potential between the styling and the pre-treating compositions (Δξ_{c-o}), in absolute terms, are at least 5 mV, at least 10 mV, at least 15 mV, at least 20 mV, at least 25 mV, at least 30 mV, or at least 40 mV, all in absolute values. In some embodiments, Δξₒ₋ₕ and Δξ_{c-o} absolute values are within a range of 5 to 80 mV, 10 to 80 mV, 10 to 70 mV, 10 to 60 mV, 15 to 80 mV, 15 to 70 mV, 15 to 60 mV, 20 to 80 mV, 20 to 70 mV, 20 to 60 mV, 25 to 80 mV, 25 to 70 mV, 25 to 60 mV, 30 to 80 mV, 30 to 70 mV, 30 to 60 mV, 35 to 80 mV, 35 to 70 mV, or 35 to 60 mV.

As it is preferred that the materials penetrating the hair shaft in priority are those participating in or promoting the in situ polymerization of the PBM(s), it can be beneficial to select the hair pre-treating composition to be substantially unable to penetrate into the hair. Hence, in some embodiments, the pre-treatment oil has a hair-penetrating ability as measured by weight gain of up to 5 wt. %, up to 4 wt. %, up to 3 wt. %, up to 2 wt. %, or up to 1 wt. % by weight of the hair fibers. Regardless of their ability to penetrate, or not, into the hair fibers, hair pre-treating compositions must not adversely interfere with the sought activity of the hair styling composition (e.g., prevent its polymerization, as can be tested in vitro).

While hair pre-treating compositions may provide a variety of beneficial effects, in some embodiments of the present Invention, one such benefit results from selecting a pre-treating oil being incompatible with the hair styling composition from a miscibility standpoint. For example, the oil can have a miscibility of 5 wt. % or less, 4 wt. % or less, 3 wt. % or less, 2 wt. % or less, or 1 wt. % or less, by weight of the hair styling composition, as measured at a temperature of 25° C. In such a case, it is believed that the hair pre-treatment composition forms a thin oily layer on the hair surface upon which excess hydrophobic droplets of hair styling composition may bead. Thus, while the thin layer of pre-treatment allows for the penetration of the components of the styling composition, its presence facilitates removing the part of the hair styling composition that did not penetrate the hair fibers. Excess styling composition may be removed along with the layer of the pre-treatment oil e.g., by washing the hair or wiping off. In such a case, a pre-treatment oil may improve the appearance, the feel, and/or the combability of the hair fibers at an earlier stage as compared to hair fibers treated with a same hair styling composition in absence of the pre-treating oil.

In some embodiments, the pre-treatment composition is an oil selected from silicone oils.

Whether any one of the afore-described optional pre-treatment steps was previously performed or not, the hair styling composition (e.g., oil-in-water emulsion) is applied to the hair fibers, and maintained on the hair typically for a period of at least 5 minutes, allowing the cuticle scales to swell and open, and thus granting to at least part of the PBMs, the WHAs and the curing facilitators, if present, access into the hair shaft. To facilitate penetration into the hair cortex, the molecules participating in or facilitating the internal polymerization or protecting a resulting effect of the polymer (e.g., PBMs, curing facilitators, WHAs, co-solvents) preferably have a molecular diameter of less than 2 nm, less than 1.8 nm or less than 1.6 nm. The Inventors posit that once within the shaft, the monomers can bond to at least part of the broken hydrogen bonds in the hair fibers, preventing them from re-forming in their prior native state upon exposure to water. The PBMs may additionally, or alternatively, polymerize without being bonded to the previously broken hydrogen bonds. Regardless of the mechanism of action, polymers resulting from the curing of the monomers having impregnated the hair fibers are able to constrain the hair fibers in their new shape. It is believed that the cured composition of the invention prevents water (either ambient or applied during wetting) from accessing the hair or diminishes its access, thereby reducing or delaying the ability of hydrogen bonds to form again, deferring the ability of the hair to revert to its native shape. The WHAs may further contribute to water sequestration, or any other interaction furthering the desired effect of the polymer with respect to styling. Regardless of the mechanism of action of the WHAs with respect to the present hair styling method, to the polymer and to the duration of its effect on styling, the WHAs are for convenience said to provide a protective effect or a prolonging effect. While for simplicity the method is described in terms of breakage of hydrogen bonds and subsequent blockage of the broken bonds by attachment to PBMs or other ingredients that may thereafter polymerize or interact with hair constituents, this is not meant to rule out any additional rationale underlying the observed styling effect.

Sufficient time is provided for the monomers to impregnate the hair fibers and ensure their bonding e.g., to at least part of the broken hydrogen bonds in the hair fibers. In some embodiments, the composition is allowed to remain in contact or is maintained applied on the hair fibers for a period of at least 10 minutes, at least 20 minutes, at least 30 minutes, at least 35 minutes, at least 40 minutes, at least 45 minutes, or at least 50 minutes. In some embodiments, the time period during which the composition remains applied on the hair fibers, alternatively referred to as the incubation time, is of at most 12 hours, at most 10 hours, at most 5 hours, at most 2 hours, or at most 1 hour. In particular embodiments, the composition is maintained on the hair fibers for a period of time between 5 minutes and 30 minutes, 10 minutes and 60 minutes, 30 minutes and 12 hours, between 30 minutes and 5 hours, between 40 minutes and 2 hours, or between 50 minutes and 2 hours. It is to be noted that conventional straightening methods may sometimes require longer period of times, some requiring 3-4 hours, or even 6-8 hours of application.

The composition can remain applied on the hair fibers at an ambient temperature, but this step can alternatively be performed at an elevated temperature of at least about 30° C., or at least about 40° C. In some embodiments, the temperature at which the composition can remain in contact with the hair fibers is of at most about 60° C., at most about 55° C. or at most about 50° C. In particular embodiments, the liquid composition is maintained on the hair fibers in a temperature range between 15° C. and 23° C., between 23° C. and 60° C., between 25° C. and 55° C., or between 25° C. and 50° C.

After said period of time, allowing for sufficient penetration of at least part of the PBMs and WHAs of the composition within the individual hair fibers, the monomers are subsequently at least partially cured, optionally in the presence of the curing facilitators, by application of energy, so as to effect at least partial polymerization.

Upon polymerization of the PBMs, as can be more readily assessed within the liquid composition than within the hair fibers, the resulting polymer develops increasing glass transition temperature (Tg). In some embodiments, upon complete curing, the resulting PBP has a Tg of at least 50° C., at least 100° C., at least 150° C., or at least 200° C. Such Tg allows the polymerized PBMs to remain intact under hot weather conditions, when washing the hair with hot water (around 45° C.), or even when being in an environment of elevated temperature, such as in a sauna (around 70° C.). As the synthetic polymer having formed within the hair fiber, thanks to its Tg, remains unaffected by such conditions or treatments, so is the modified shape of the hair achieved using the compositions and methods according to the present teachings.

In some embodiments, the energy allowing for at least partial curing of the composition (hence styling of the hair fibers) is a thermal energy, applied at a temperature of at least about 80° C., at least about 100° C., at least about 120° C. or at least about 140° C. In some embodiments, the heating temperature is at most 220° C., or at most 200° C. In particular embodiments, the temperature applied to achieve at least partial curing is in a range between 80° C. and 220° C., between 100° C. and 220° C., between 120° C. and 220° C., or between 140° C. and 200° C. It should be appreciated that the temperature provided by a heating device in order to at least partially cure the monomers is generally higher than the temperature perceived by the hair fibers. While given a long enough residence time (period during which the hair segment is exposed to the heat), the temperature of the hair fiber could eventually reach the temperature of heating, this is not generally the case and the temperature of the hair fibers at which curing may take place is typically of at least about 45° C., at least about 50° C., at least about 55° C., or at least about 60° C. In order to prevent irreversible damage to the hair fibers, the temperature of the hair fibers during the at least partial curing step is desirably of no more than 180° C., no more than 140° C., or no more than 100° C. The at least partial curing can be effected while styling the hair into the desired shape, e.g., by a hair dryer, or a flat or curling iron, so as to modify the native shape. This step, during which the hair fibers are mechanically constrained in a dynamic or static way to modify their shape (e.g., being pulled over a comb or brush, rolled on a roller, or contacted by a styling iron), can therefore alternatively be referred to as the styling step.

The time needed to reach at least partial curing at such temperatures is generally brief. Typically, an area of individual hair fibers perceiving a temperature of 100° C. or more may locally provide the partial polymerization of PBMs therein within a few seconds, whereas hair fibers reaching a lower temperature of about 50° C. may require up to a few minutes (e.g., five minutes). The duration of time hair should be subjected to heating, hence should be perceiving a particular temperature adapted for curing, may depend on the shape of the hair to be modified and the new shape to be formed. A relatively mild modification may require less time than a relatively more dramatic change of shape.

A duration of time during which hair fibers should be at a suitable temperature can be independently tested in vitro by subjecting the oil phase of the composition due to be dissolved or emulsified to a temperature intended for the hair treatment, measuring the time it takes for the liquid phase to start solidifying (i.e., curing). When considering a mammalian subject, the amount of time allocated for the partial curing step (in other words, for the styling of the hair per se) would depend inter alia on the type of hair, its density on the scalp and its length, as well as on the device used to deliver the heat and its degree. Hence, on the level of an entire hair scalp, partial curing may take a few minutes, but generally no more than an hour. Such considerations apply to any other treatment of the hair fibers, the duration of time provided herein generally referring to periods suitable to any amount of hair fibers that can be simultaneously treated. If an entire hair scalp is to be treated step-wisely by repeating a same treatment for different batches of hair fibers, then the duration of treatment for the entire scalp may amount to the sum of durations due for the actual number of individual repeats of simultaneous treatments. For illustration, if five minutes are required to simultaneously treat a first batch of hair fibers, and an entire hair scalp is constituted of four such batches, then the treatment will be completed within about 20 minutes.

Prior to the at least partial curing, excesses of the liquid composition are optionally removed from the outer surface of the hair fibers by rinsing the fibers with a rinsing liquid, so as to prevent formation of a thick coating on the surface of the hair fibers, and thus avoiding a tacky and coarse feel to the hair. The removal of such coating can be further facilitated by the application of a suitable pre-treatment composition, such as an oil pre-treatment, as previously described. Rinsed fibers may also display improved heat transfer, accelerating partial curing therein.

Alternatively, or additionally, following the application of the hair styling composition and its incubation on the hair fibers, and optionally following rinsing, but prior to hair styling, a second composition consisting of curing facilitators can be applied to the hair fibers impregnated with the PBMs. The composition that may be used in this optional step can be referred to as a curing composition. It may contain the same curing facilitators, selected from cross-linkers and curing accelerators previously described for the hair styling composition, and typically the curing composition consists of curing accelerators. In contrast with the hair styling composition, the curing facilitators (e.g., the curing accelerators) can be present in the curing composition in excess amount (e.g., at 5 wt. %) allowing the application of the curing composition to the hair fibers to be relatively brief (e.g., between 5 and 15 minutes, or less). The curing composition may additionally serve to rinse the fibers in addition to or instead of a rinsing solution.

Following the at least partial curing, sufficient to achieve the desired modified shape, the hair fibers may optionally undergo further curing by application of further energy, preferably heat, to ensure additional curing of the composition. The further energy can be applied by the use of the above-mentioned styling instruments, e.g., hair dryer, or styling iron. In some embodiments, the further curing can be performed at temperatures as described for the at least partial curing of the third step, typically for a duration of time significantly longer than for partial curing. For instance, if hair fibers treated with a composition enabling at least partial curing with a specific styling device at a predetermined temperature within 20 minutes (as established by the fibers of the entire scalp displaying the desired modified shape), then an optional additional heating step favoring further curing would be performed for at least 40 minutes at least under the same conditions. Whereas partial curing is achieved while modifying the shape of the fibers, the step referred to herein as further curing is applied once the hair fibers are in the desired modified shape, so that concurrent mechanical constraining of the fibers to adopt the desired shape is no longer necessary. While further curing is expected to increase the extent of polymerization of the PBMs within the hair fibers, it is not anticipated to achieve full curing (e.g., following which, polymerization can no longer take place).

In some embodiments, after heat curing (e.g., achieved during the styling step and the optional further curing), the hair fibers can be maintained, unwashed, to reduce exposure to water, allowing curing to further proceed, if applicable. The period during which washing of the hair fibers can be avoided may depend on the type of hair, the composition applied thereto, the procedure used to modify the native shape, the temperature, the relative humidity, the desired modified shape and the desired duration of said modification. Typically, assuming the hair fibers are maintained at room temperature at a relative humidity of about 40-60RH %, washing of the hair may take place at least 18 hours after the termination of the at least partial curing (e.g., styling including mechanical constraint) or optional further curing step (e.g., heating without mechanical constraint). In some cases, washing can be deferred for at least 24 hours, for at least 36 hours, or for at least 48 hours. Usually, washing of hair styled according to the present method takes place within at most a week from styling. Hair styled according to the invention can be washed with any shampoo, not being restricted to the use of a particular one to avoid ruining the styling effect, as often necessary for conventional methods. Nevertheless, regular shampoos can be improved by including curing facilitators.

Advantageously, hair treated by the present hair styling compositions and the according methods is not only relieved of ongoing particular care, but the present teachings can also be suitable for hair fibers that have previously undergone other hair treatments (e.g., bleaching, coloring, styling, etc.). Such conventional treatments generally damage the hair, inflicting structural changes, e.g., physical and/or chemical, that might hamper subsequent hair treatment, such as styling by traditional methods (e.g., organic or Japanese). For instance, bleached hair might not be effectively straightened by the Japanese method due to the bleaching chemicals affecting hair components necessary for this method. In contrast, the compositions of the present invention are able to effectively style hair fibers regardless of any previous hair treatment they might have undergone.

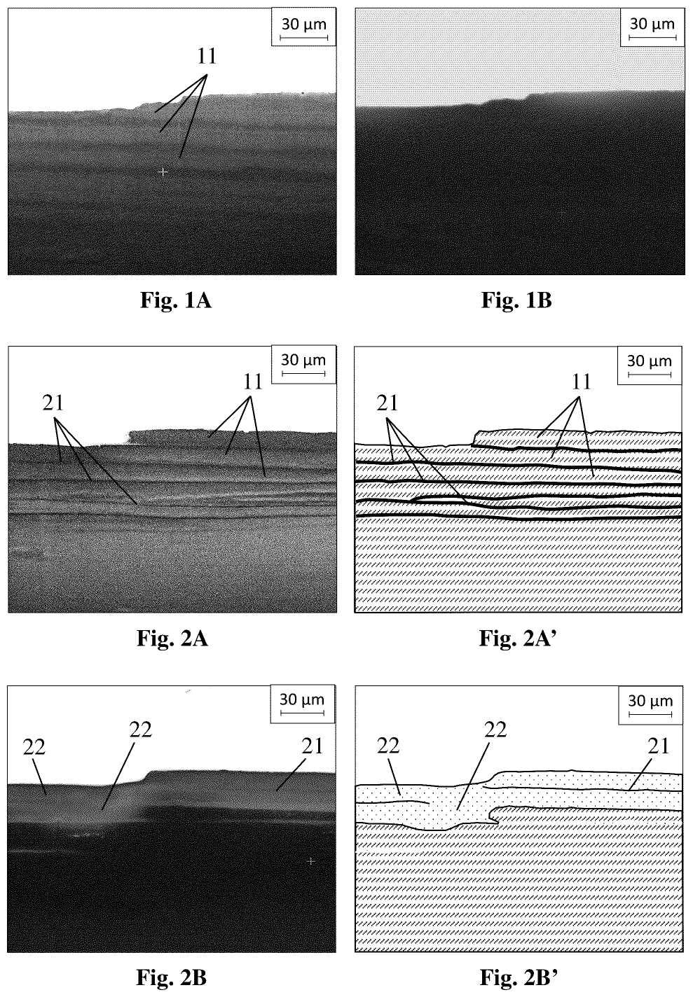

FIGS. 1A and 1B show FIB-SEM images of a hair fiber washed with tap water containing 5% sodium lauryl sulfate to remove any residual materials adhered to the hair and yield a better visualization of the cuticle scales of a reference untreated hair fiber. FIG. 1A, showing cuticle scales 11 layered one on top of the other, was captured by a scanning electron microscope (SEM) and by focused ion beam (FIB) measurements, performed on a cross-section of a hair fiber, using Zeiss Crossbeam 340 microscope. The cross section was performed with ionized gallium bombarding the sample at 30 kV and 100 pA, at an angle of 540 from the SEM column, its image was taken at a magnification of x20K, at a voltage of 1.20 kV, and at a working distance of 5 mm, with the SEM column and an in-lens detector. FIG. 1B is another FIB-SEM image of the same hair fiber, but at a working distance of 4.9 mm and a voltage of 10 kV (cuticle scales are not visible at this voltage).

FIGS. 2A and 2B show FIB-SEM images, captured as described above, of a hair fiber treated with an oil-in-water emulsion of the present invention (specifically, emulsion PU6, prepared as described in Example 3), immediately after styling of the hair fibers as described in step 4 of Example 5, i.e., before step 5 of washing the hair fibers. The images were captured at two different voltages, the lower one enhancing the visibility of the cuticles and their profile, whereas the higher voltage enhanced the visibility of the composition. As the images were taken on a same cross-section, they can be “superimposed” one on the other to combine the information gathered at each voltage. FIG. 2A was taken at a voltage of 1.20 kV and a working distance of 5 mm, and the cuticles 11 can be seen layered one on top of the other, separated by dark lines 21 possibly indicative of cuticle-cuticle cell membrane complex (CMC). The cured hair styling composition cannot be seen at this voltage. To better illustrate the hair structure, a schematic depiction of FIG. 2A is provided in FIG. 2A′. FIG. 2B presents an image of the same hair fiber but captured at a voltage of 10 kV and a working distance of 4.9 mm, whereby the cuticle outlines fade out but the cured composition becomes visible as bright layers 22, which can be seen as having penetrated through a few layers of cuticles into the hair fiber. FIG. 2B′ is a schematic depiction of FIG. 2B, wherein the cured composition is shown as sparsely dotted white areas within the hair fiber (itself, presented as a slashed area).

The methods of the present invention provide for durable hair styling, which keeps the hair fibers in the desired shape even after the hair is exposed to moisture—whether to water originating from the atmosphere humidity or following wetting or washing of the hair. The hair styling can be maintained for long periods of time, wherein the styled shape is not affected in a significantly detectable manner even after 5 shampoo washes or more. As shall be demonstrated with the working examples, in some embodiments the hair styling composition and method according to the present teachings provide long lasting modification of the hair shape, as evidenced by the ability of the treated hair to withstand 10 or more shampoo washes, 20 or more shampoo washes, 30 or more shampoo washes, 40 or more shampoo washes, or 50 or more shampoo washes.

FIGS. 3A and 3B are FIB-SEM images of a hair fiber treated by application of emulsion PU6, straightened and thereafter washed for 16 washing cycles, the straightening and washing procedures being respectively described in Examples 4 and 5. The images were captured as described above, wherein FIG. 3A shows a cross-section taken at a voltage of 1.20 kV and a working distance of 4.7 mm, and FIG. 3B shows the same cross-section of the styled hair fiber, the image being taken at a voltage of 10 kV and a working distance of 4.6 mm. FIGS. 3A and 3B demonstrate the presence of the cured composition within the hair fiber, even after 16 washing cycles.

While it cannot be ruled out that part of this “wash resistance” results from residual disseminated coating on the fibers' outer surfaces, the Inventors posit that as such an external coating tends to wear out relatively rapidly with washes, and the ability to style hair according to the present teachings can be attributed predominantly to the internal polymerization of the PBMs. It is to be noted that this transient scattered coating is relatively thin, usually not exceeding an initial thickness of 1 μm, often being less than 0.5 μm thick, which in itself distinguishes hair fibers treated according to the present teachings from conventional styling methods relying on continuous external coatings of a few microns to constrain the fibers in a desirable shape. Without wishing to be bound by theory, it is believed that this transient thin coating of the hair fibers may temporarily protect the inner shaft so that the monomers having penetrated therein can further their curing, strengthening their polymerization, thus extending the hair styling durability. As exemplified hereinbelow, the styling of hair according to the present method is maintained in absence of the transient coat, the avoidance of which can be facilitated by application of an oil pre-treatment to the hair fibers.

As used herein, a composition providing for a modified shape able to resist 5 to 9 shampoo washes can be referred to as having a short-term styling effect. A composition providing for a wash resistance of 10-49 shampoo cycles is said to provide for a semi-permanent styling, whereas compositions providing wash resistance to more than 50 shampoos can be said to provide permanent styling.

The rapid absence of a continuous external coat (insignificant for the present long lasting styling effects) is deemed advantageous, as methods relying on such peripheral constricting structures to durably maintain a straightened hair shape have often been found detrimental to hair health and natural look.

FIG. 4A shows a natural, untreated curly black hair tuft, in which twists (e.g., dips 42 and peaks 44) in the hair fibers are clearly detectable. A sample of similar hair, treated with a hair styling composition described in the present invention as PU6 and straightened with a flat iron, is shown for comparison in FIG. 4B. As can be seen, the treated hair fibers show a drastic decrease in the number of twists as compared to the untreated reference.

While the present compositions and methods are particularly beneficial for long lasting hair styling, for which the alternatives are typically deleterious to the hair and often to the health, they may additionally or alternatively be used for short-term hair styling, the hair fibers regaining their native original shape following 2 to 4 shampoo washes.

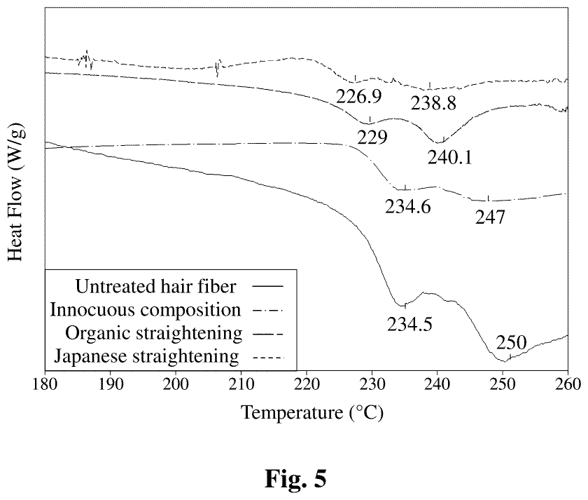

FIG. 5 depicts the results of a DSC study showing that traditional hair straightening methods are damaging to the hair and illustrating the impact expected from an innocuous hair styling method, as anticipated for the hair styling compositions of the present invention. As can be seen in the figure, the curve of a sample of hair fibers as would appear if treated with a composition of the present invention can be comparable to the curve of untreated, native hair sample, indicating no significant structural changes, hence damage to the hair. In contrast, the DSC curves of commercial hair straightening methods (organic and Japanese) show substantial changes from the native hair sample curve, indicating structural changes, which are to be expected when using such drastic hair styling methods. The DSC study is further detailed in Example 11 below.

Advantageously, hair fibers treated by the compositions according to the present teachings are expected to display at least one endotherm temperature within 4° C., within 3° C., within 2° C., or within 1° C. from similar untreated fibers, as measured by thermal analysis.

The non-damaging effect of the present compositions to hair fibers treated therewith can be confirmed or alternatively established by tensile testing, wherein various mechanical parameters can be compared between treated and untreated hair fibers, as described in Example 12 below. While fibers styled using conventional organic straightening are expected to show inferior mechanical properties compared to untreated fibers, fibers treated according to the present invention may display behavior similar or even superior to untreated fibers of similar nature. Without wishing to be bound by any particular theory, such improved properties, or at least absence of significant deterioration, are believed to stem from the presence of a polymerized version of the PBMs within the inner parts of the hair fibers.

One mechanical parameter, where hair fibers treated by the present invention are expected to be at least as good as untreated hairs relates to the pressure (or force per cross-sectional area) required to break the hair, or break stress, measured at the break point in a strain-stress curve. A second mechanical parameter is hair toughness, which estimates the amount of energy the hair can absorb before breaking (i.e., the area under the strain-stress curve). Elastic modulus is another mechanical parameter, indicating the hair fibers' resistance to elastic deformation, where fibers treated by the present methods are expected to be at least comparable to untreated hair.

In some embodiments, the hair fibers treated by the compositions according to the present teachings, when measured by tensile properties analysis, display at least one of:
a break stress of at least 5%, at least 10%, at least 20% or at least 25% greater than the break stress of similar untreated fibers; and
a toughness of 95% or more, 100% or more, 105% or more, 110% or more, 115% or more, or 120% or more of similar untreated hair fibers.

The methods of the present invention are suitable for any desired hair style and shape, such as straightening, curling, or rendering an intermediate shape, wherein the hair is relaxed to a form less wavy than its natural unmodified shape.

Advantageously, the present compositions allow restyling without necessitating application of a new composition. Hence, following a single pass of the method, embodiments of which have been described above, the method serving to modify the shape of the hair fibers from a native shape to a first modified shape, the hair fibers can be reshaped to a second modified shape. This can be achieved by bringing the hair fibers to a temperature above the Tg or softening temperature of the polymer formed during the first shaping, hence affording what may be referred to as “at least partial softening”. During and/or following such a step of at least partial softening, the hair fibers are formed in a desired second shape. The polymer is then allowed to regain a constraining structure adapted to retain the second shape, by allowing the temperature to decrease below its Tg or softening temperature while the hairs are maintained in the desired shape. The temperature can alternatively be actively lowered, for instance by blowing cool air on the hair. The second modified shape can be the same or different than the first modified shape. While this innovative restyling method has been described with respect to the softening of the polymer having previously penetrated within the fibers, it is believed that the heating applied to achieve such softening may additionally serve to decrease the water content. As previously explained, the elimination of residual water may, in turn, affect hydrogen bonding, enhancing the effect of the polymer having reformed upon cessation of its softening.

Advantageously, the present compositions allow “de-styling” when desired, by which it is meant that the hair fibers treated according to the present invention can regain their original shape without waiting for the effect of styling to vanish with time or for the regrowth of naturally shaped hair fibers. This can be achieved by subjecting the previously styled hair fibers to a temperature above the Tg or softening temperature of the polymer in the presence of water for a sufficient amount of time for the temperature to soften the polymer, and the water to penetrate the fibers. Without wishing to be bound by theory, it is believed that that such de-styling treatment could result in the softening of the polymer, thus possibly allowing a certain degree of cleavage of bonds that the polymer may have formed with moieties of the hair fibers prone to form hydrogen bonding. The presence of water during the de-styling treatment enables penetration of such molecules into the hair, resulting in the reformation of at least part of the hydrogen bonds naturally occurring in the untreated hair. Depending on the extent of reformation of the original hydrogen bonds of the hair fibers, and the form the polymer may adopt upon cooling back to a lower temperature no longer supporting its softening, the de-styling can be partial or complete, the hair accordingly returning less or more closely to its original shape. The de-styling process is believed to only affect the shape of the polymers remaining within the hair shaft, therefore, following de-styling, the hair fibers can, if desired, undergo an additional styling treatment, as previously described for restyling.

The Tg or softening temperature of the synthetic polymer within the hair fibers can be empirically assessed, for example in vitro. A sample of the hair to be restyled or de-styled can be collected from the hair scalp to be treated by such methods and placed in the intended re-/de-styling liquid (e.g., water). At this stage, the hair fibers of the sample have a particular modified shape. Temperature can be gradually raised and the ability of such temperature to relax the shape monitored. A temperature is deemed suitable for the at least partial softening of the polymer when the hair fibers lose their modified shape and revert towards their native shape. A suitable temperature may also depend on the duration of the sample incubation. Alternatively, the Tg of a phenol-based polymer (PBP) formed by polymerizing in vitro the phenol-based monomers (PBMs) according to the method previously described can be determined by standard thermal analysis methods, e.g., DSC, such as described in ASTM E1356. In some embodiments, the Tg or softening temperature of the polymer is at least 40° C., at least 50° C., or at least 60° C., such softening temperature generally not exceeding 80° C. The duration of time the hair fibers should be subjected to such temperatures to achieve restyling or de-styling can be similarly determined. Typically, such treatments last at least 5 minutes, at least 10 minutes, at least 20 minutes, at least 30 minutes, at least 40 minutes, at least 50 minutes, or at least 60 minutes, generally not exceeding 4 hours or 3 hours, relatively higher temperatures requiring relatively shorter softening times. The hair styling compositions can be sold with guidance concerning the temperature and time needed to effect restyling or de-styling if desired.

Advantageously, the present compositions and methods are suitable for the styling of growing hair. The synthetic polymer formed by a first application of the hair styling composition is expected to be located in the segments of the hair fibers available above scalp at the time of application of the monomers. With time and hair growth, such segments are to be found more and more distal from the scalp, while the newly grown hair segments adjacent to the scalp would be devoid of such inner styling skeleton. It is believed that hair styling compositions applied at a later time following such hair growth would probably act mainly on the newly grown segments, the earlier treated segments being already “occupied” by previously formed synthetic polymer and crystallized WHAs. However, since as explained the existing polymer can permit restyling or de-styling of the fibers, it may functionally merge with a polymer that would be newly formed in the new segments, providing a “styling continuity” along the entire fiber, preexisting and newly grown.

The present invention further provides a liquid composition for styling mammalian hair fibers, wherein the liquid composition is a curable single-phase composition comprising:
at least one PBM, as herein described;
at least one WHA, as herein described;
water; and
one or more co-solvents;
the liquid composition having a pH adapted to facilitate the penetration of at least part of the PBM(s) and the WHA(s) within the hair fibers.

The present invention further provides a liquid composition for styling mammalian hair fibers, wherein the liquid composition is a curable oil-in-water emulsion comprising:
an oil phase containing at least one PBM, as herein described; and
an aqueous phase containing at least one WHA, as herein described, and water, the water and/or the aqueous phase being at a pH adapted to facilitate the penetration of at least part of the PBM(s) and the WHA(s) within the hair fibers;
each of the oil phase and the aqueous phase optionally further comprising one or more co-solvents; and
the oil phase being dispersed within the aqueous phase.

In some embodiments, the single-phase composition or the oil-in-water emulsion optionally further contains at least one curing facilitator selected from a cross-linker and a curing accelerator, as described above and further detailed herein.

In some embodiments, the liquid hair styling composition (e.g., oil-in-water emulsion) optionally further contains at least one additive, selected from a group comprising an emulsifier, a wetting agent, a thickening agent, an auxiliary polymerization agent and a charge modifying agent, as described above and further detailed herein.

Advantageously, the hair styling compositions according to the present teachings are devoid of known carcinogenic compounds. For instance, in some embodiments, the hair styling composition contains permissible trace amounts of such compounds, which depending on jurisdiction can be less than 0.5 wt. % formaldehyde, less than 0.2 wt. % formaldehyde, less than 0.1 wt. % formaldehyde, or even below permissible regulatory levels of less than 0.05 wt. % formaldehyde, less than 0.01 wt. % formaldehyde, less than 0.005 wt. % formaldehyde, less than 0.001 wt. % formaldehyde, or no formaldehyde, by weight of the composition. The same limited concentrations apply to products that may produce or act as formaldehyde (e.g., glyoxylic acid and its derivatives, or any other formaldehyde-releaser), to glutaraldehyde and to products that may produce or act as glutaraldehyde (e.g., 2-alkoxy-3,4-dihydropyran). These deleterious compounds, including their respective precursors or substituted forms (also termed formaldehyde-producing compounds or -releasers), such as Quaternium-15 (including for instance Dowicil™ 200; Dowicil™ 75; Dowicil™ 100; Dowco™ 184; Dowicide™ Q produced by Dow Chemical Company); imidazolidinyl urea (such as Germall™ 115 Ashland); diazolidinyl urea (such as Germall™ II); bromonitropropane diol (Bronopol); polyoxymethylene urea; 1,2-dimethylol-5,6-dimethyl (DMDM) hydantoin (traded as Glydant); tris(hydroxymethyl) nitromethane (Tris Nitro); tris(N-hydroxyethyl) hexahydrotriazine (Grotan® BK); and sodium hydroxymethylglycinate), can be referred to herein, individually and collectively, as small reactive aldehyde(s) (SRA(s)).

As appreciated by persons skilled in organic chemistry, SRA molecules need not be aldehyde per se and can be of additional chemical families as long as being able to form (e.g., by hydrolysis, degradation, reaction, and the like) deleterious aldehydes including formaldehyde and glutaraldehyde. Such formation can be triggered by conditions often encountered in hair styling, such as upon application of heat. Some of such precursors can entirely convert into formaldehyde or glutaraldehyde, one molecule of SRA yielding, optionally via intermediate products, one or more molecules of formaldehyde under ideal conditions, which may be extreme, whereas other precursors may convert only in part. Heximinium salts are one example of the latter.

In any event, assuming the SRA compounds are other than formaldehyde or glutaraldehyde, their weight amount in the composition would exceed the final weight amount of formaldehyde or glutaraldehyde that can be formed thereby. In particular embodiments, the hair styling composition contains less than 0.5 wt. % SRA, less than 0.2 wt. % SRA, less than 0.1 wt. % SRA, less than 0.05 wt. % SRA, less than 0.01 wt. % SRA, less than 0.005 wt. % SRA, less than 0.001 wt. % SRA, or no SRA, by weight of the composition. As can be appreciated, the hair styling composition will be deemed to be essentially free of SRA molecules if containing or producing during the hair styling method (e.g., upon heating of the composition) undetectable levels of formaldehyde.

As formaldehyde reacts with hair proteins, its substantial absence from the present hair styling compositions results in a corresponding absence of its reaction products in the treated hair fibers. Reaction products of formaldehyde depend on the amino acid it is reacting with, and, by way of example, reaction with cysteine yields thiazolidine and hemithioacetal, reaction with homocysteine yields thiazinane and hemithioacetal, reaction with threonin yields oxozolidine, and reaction with homoserine yields 1,3-oxazinane. Such reaction products can be detected in hair fibers by standard methods, including by NMR.

Thus, mammalian hair fibers styled according to the present methods, or with the present compositions, can be characterized by containing less than 0.2 wt. %, less than 0.1 wt. %, less than 0.05 wt. %, less than 0.01 wt. %, less than 0.005 wt. %, less than 0.001 wt. %, or being significantly devoid of reaction products between formaldehyde and amino acids. In some embodiments, the mammalian hair fibers treated according to the present teachings contain undetectable levels of at least one of thiazolidine, hemithioacetal, thiazinane, oxozolidine, and 1,3-oxazinane, as can be measured by NMR. As cysteine may account for up to 18% of the amino acid repeats of normal human keratin protein, the absence of thiazolidine and/or hemithioacetal in the hair fibers might be the most significant marker(s) for the corresponding absence of formaldehyde and formaldehyde forming products in the composition previously used to treat the hair.

In some embodiments, the hair styling composition is substantially devoid of amino acids, peptides and/or proteins. Proteins absent from the present compositions can be naturally occurring proteins, such as keratin and collagen, or synthetic and/or modified (e.g., hydrolyzed) forms thereof, and the lacking peptides may be smaller fragments of such proteins. For simplicity, such peptides may be named according to the larger protein they may be part of, and for instance can be referred to as keratin-related peptides or collagen-related peptides, when considering the proteins most frequently used in hair treatment.

The compositions according to the present invention are substantially devoid of such substances, if amino acids, peptides or proteins, and in particular keratin, collagen and their related peptides, constitute no more than 1 wt. % of the composition, their respective concentration being preferably of no more than 0.5 wt. %, of no more than 0.1 wt. %, or of no more than 0.05 wt. % by weight of the hair styling composition. In some embodiments, such substances are substantially absent (e.g., at about 0 wt. %) from the composition, accordingly. The presence or absence of such biomolecules can be determined by standard methods, for example by matrix-assisted laser desorption/ionization (MALDI) and related techniques, including for instance with a time-of-flight mass spectrometer (MALDI-TOF).

Thus, mammalian hair fibers styled according to the present methods, or with the present compositions, can be additionally or alternatively characterized by being significantly devoid of peptides and proteins, other than naturally formed ones. If the hair fibers were treated by a conventional method using naturally occurring proteins or related peptide fragments thereof, then hair fibers styled according to the present methods can in contrast be characterized by being significantly devoid of peptides of proteins naturally occurring in the hair fibers.

The compositions according to the present invention and mammalian hair fibers styled therewith can be additionally or alternatively characterized by the presence of WHA in the composition or within the hair fibers, which can be determined by any method suitable for the WHA being considered.

When detection of a material within the hair fibers is sought, the hair fibers are typically thoroughly washed and rinsed (e.g., at least ten times) with a cleaning product devoid of the material under consideration to ensure that any level detected following extraction stems only from the inner part of the hair fiber. For illustration, when the material to be detected is the WHA, and the one used in the present hair styling compositions or methods is urea, the cleaning products used to wash the hair fibers would be devoid of urea, and following sufficient extraction (e.g., of up to 12 hours in a suitable liquid, such as water, preferably at elevated temperature, such as 70° C.), the presence of urea in extracts obtained from samples of treated hair fibers can be detected by Electrochemical Impedimetric Spectroscopy (EIS), X-Ray Diffraction (XRD), or by standard laboratory methods, commonly used for medicinal purposes.

Identification of a hair styling compositions of the present invention can be also performed by detecting functional groups characteristic of the essential components of the composition or in extracts from hair styled fibers, such functional groups such as phenol, which can be detected by any method known in the art, such as Fourier Transform Infrared Spectroscopy (FTIR).

In summary, mammalian hair fibers comprising in their inner part at least partially cured PBMs of the present invention, forming a synthetic polymer within the fiber, can be characterized by at least one of the following features:
having less than 0.2 wt. % of a reaction product of formaldehyde and amino acids, the reaction product being selected from a group comprising thiazolidine, hemithioacetal, thiazinane, oxozolidine, and 1,3-oxazinane thiazolidine, by weight of the hair fibers;
displaying at least one endotherm temperature within 4° C., within 3° C., within 2° C., or within 1° C. from untreated hair fibers, as measured by thermal analysis such as DSC;
having a break stress of at least 5%, at least 10%, at least 20% or at least 25% greater than the break stress of similar untreated fibers, as measured by tensile analysis;
having a toughness of 95% or more, 100% or more, 105% or more, 110% or more, 115% or more, or 120% or more of similar untreated hair fibers, as measured by tensile analysis;
having less than 0.2 wt. %, less than 0.1 wt. %, less than 0.05 wt. %, less than 0.01 wt. %, less than 0.005 wt. %, or less than 0.001 wt. % of small reactive aldehydes (SRA) selected from: formaldehyde, formaldehyde-forming chemicals, glutaraldehyde, and glutaraldehyde-forming chemicals, by weight of the hair fibers; and
displaying a signal indicative of the presence of a WHA, such as an Electrochemical Impedimetric Spectroscopy (EIS) signal indicative of the presence of urea.

In one embodiment, the mammalian hair fibers fulfill at least feature i) as above listed; at least feature ii) as above listed at least feature iii) as above listed; at least feature iv) as above listed; at least feature v) as above listed; or at least feature vi) as above listed. In one embodiment, the mammalian hair fibers fulfill at least features i) and ii) as above listed; at least features i) and iii) as above listed; at least features i) and iv) as above listed; at least features i) and v) as above listed; at least features i) and vi) as above listed; at least features iii) and iv) as above listed; at least features i), iii) and iv) as above listed; at least the features i), ii), iii), and iv) as above listed; at least the features i), ii), iii), iv) and v) as above listed; or at least the features i), ii), iii), iv), v) and vi) as above listed.

The present invention also provides a kit for styling mammalian hair fibers, the kit comprising:
a first compartment containing at least one PBM; and
a second compartment containing at least one WHA and at least one of:
   1) water;
   2) a co-solvent; and
   3) a pH modifying agent;
wherein the contents of the second compartment are a liquid having a pH selected to increase the penetration of at least part of the monomer into the hair fibers;
wherein the mixing of said compartments' contents produces the hair styling composition (e.g., single-phase or oil-in-water emulsion) described above and further detailed herein.

In some embodiments, the components of the kit are packaged and kept in the various compartments under an inert environment, preferably under an inert gas, e.g., argon or nitrogen, and/or under any other suitable conditions preventing or reducing during the storage of the kit adverse reactions that may diminish efficacy of the composition. For instance, the kit should be stored at temperatures that would not induce polymerization, such as below 30° C., below 27° C. or below 25° C.

In some embodiments, the at least one PBM is pre-polymerized prior to its placing in the kit's first compartment.

The kit may further comprise at least one curing facilitator, being a condensation-curable cross-linker or an addition-curable cross-linker. The curing facilitator may also be a curing accelerator as described above, used to facilitate the polymerization. The curing facilitator (being a cross-linker or a curing accelerator) may be placed in the first or second compartment, depending on its reactivity with any one of the components of these compartments. For example, polyamines cross-linkers do not react with the PBM at room temperature, and therefore can be contained in the first compartment. Alternatively, if the curing facilitator tends to spontaneously react with any one of the components, it may be placed in a separate additional compartment. A reactive silane cross-linker is such an example, where its placing in the same compartment as the PBM, would result in their reaction, even at room temperature, and therefore it will be placed separately in the kit.

The kit may optionally further contain at least one of a co-solvent, an emulsifier, a wetting agent, a thickening agent, an auxiliary polymerization agent and a charge modifying agent, as previously detailed, which can be included in any one of the compartments described above, or in separate additional compartments. When considering the placement of such additives, oil-soluble components are preferably placed in compartments containing mostly oily components (e.g., the first compartment), and water-soluble components are preferably placed in compartments containing mostly aqueous components (e.g., the second compartment).

The kit typically includes a leaflet guiding the end-user on the manner of mixing the various compartments, the order of which may depend on the nature of the ingredients and/or the contents of the respective compartments. Generally, the proposed method of mixing and application shall enable the preparation of an effective and safe composition, to be applied within a time period suitable for its potency and intended use. For instance, if a third compartment containing a silane derivative as a curing facilitator is included in the kit, the leaflet may indicate first mixing of the curing facilitator with the PBMs, then adding the contents of the aqueous compartment. Conversely, if a curing facilitator is present but is not a silane derivative, it may be included in the first compartment rendering the need for a separate third compartment superfluous.

In some embodiments, the ingredients of the various compartments are mixed, as may be instructed in such a leaflet, prior to the application of the final hair styling composition on the hair fibers. In such a case, the obtained composition may be used immediately, or maintained, un-applied, for up to 3 hours, up to 2.5 hours, up to 2 hours, up to 1.5 hours or up to 1 hour, prior to its application on the hair fibers.

Similarly, different timing and duration for application of the oil-in-water emulsion may conceivably be suggested depending on the desired duration of styling. For instance, if a short-term styling is desired, the composition may be applied relatively later and/or for a shorter period of time than when a longer lasting styling is desired.

### EXAMPLES

### Materials

The materials used in the following examples are listed in Table 1 below. The reported properties were retrieved or estimated from the product data sheets provided by the respective suppliers. Unless otherwise stated, all materials were purchased at highest available purity level. N/A indicates that information is not available.

TABLE 1

| | | | | |
|---|---|---|---|---|
| | Product | MW | | |
| Chemical Name | Name | [g/mol] | Supplier | CAS No. |

Phenol-Based Monomers (PBM)

| | | | | |
|---|---|---|---|---|
| Phenyl 2- | Phenyl | 214.22 | Sigma- | 118-55-8 |
| hydroxybenzoate | salicylate | | Aldrich, | |
| | (PS) | | USA | |
| 2-Ethylhexyl 2- | 2-Ethylhexyl | 250.33 | Sigma- | 118-60-5 |
| hydroxybenzoate | salicylate | | Aldrich, | |
| | (EHS) | | USA | |

Cross-Linkers

| | | | | |
|---|---|---|---|---|
| 3-aminopropyl- | Dynasylan ® | 221.4 | Evonik, | 919-30-2 |
| triethoxysilane | AMEO | | Germany | |
| | (AMEO) | | | |
| (C₇H₁₁O₂)₈ | Glycidyl | 1337.88 | Hybrid | 68611-45-0 |
| (SiO_{1.5})₈ | POSS ® Cage | | Plastics, | |
| | Mixture | | USA | |
| | EP0409 | | | |
| (C₆H₁₁O₂)₈ | Glycidyl | 1433.97 | | 160185-24-0 |
| (SiO_{1.5})₈ | Methacryl | | | |
| | POSS ® Cage | | | |
| | Mixture | | | |
| | MA0735 | | | |

Co-Solvents

| | | | | |
|---|---|---|---|---|
| Isopropyl alcohol | Isopropyl | 60.1 | Sigma- | 67-63-0 |
| | alcohol | | Aldrich, | |
| | (IPA) | | USA | |
| 2-Butanol | 2-Butanol | 74.1 | Sigma- | 78-92-2 |
| | | | Aldrich, | |
| | | | USA | |

Auxiliary Polymerization Agents

| | | | | |
|---|---|---|---|---|
| Dewaxed shellac | Dewaxed | N/A | Shellac.net | N/A |
| (beige) | shellac | | | |
| Dibutyl maleate | DBM | 228.28 | Sigma- | 105-76-0 |
| | | | Aldrich, | |
| | | | USA | |
| Oleyl amine | Oleyl amine | 267.49 | Acros, | 112-90-3 |
| | | | Belgium | |
| α-Linolenic acid | α-Linolenic | 278.4 | Antai | 463-40-1 |
| | acid | | Chemicals, | |
| | | | China | |

Additives

| | | | | | |
|---|---|---|---|---|---|
| Accelerators | Benzoyl | BPO | 242.23 | Sigma- | 94-36-0 |
| | peroxide | | | Aldrich, | |
| | | | | USA | |
| | Aluminum | Al(O-sec- | 246.32 | Sigma- | 2269-22-9 |
| | tri-sec- | Bu)₃ | | Aldrich, | |
| | butoxide | | | USA | |
| Lubricant | Cetyl | Silwax ® | N/A | Siltech, | 191044-49-2 |
| | dimethicone | B116 | | | |
| | | Silwax ® | | Canada | |
| | | H416 | | | |
| Water-soluble | Carbamide | Urea | 60.06 | Acros, | 57-13-6 |
| hygroscopic | | | | Belgium | |
| agent | | | | | |
| Thickeners | Acrylates/ | Carbopol ® | N/A | Lubrizol, | N/A |
| | C₁₀-C₃₀ | Ultrez 20 | | USA | |
| | alkyl | polymer | | | |
| | acrylate | | | | |
| | crosspolymer | | | | |
| | Guar gum | Jaguar ® | N/A | Solvay, | 39421-75-5 |
| | derivative | HP-105 | | Belgium | |
| | Mixture of | Sepiplus ™ S | N/A | Seppic, | N/A |
| | hydroxyethyl | | | France | |
| | acrylate/ | | | | |
| | sodium | | | | |
| | acryloyl | | | | |
| | dimethyl | | | | |
| | taurate | | | | |
| | copolymer | | | | |
| | and poly- | | | | |
| | isobutene | | | | |
| | and PEG-7 | | | | |
| | trimethylol- | | | | |
| | propane | | | | |
| | coconut | | | | |
| | ether | | | | |

pH modifying agents

| | | | | |
|---|---|---|---|---|
| Ammonium | NH₄OH | 35.05 | Sigma- | 1336-21-6 |
| hydroxide | | | Aldrich, | |
| | | | USA | |

Pre-treatment oils

| | | | | |
|---|---|---|---|---|
| Distearmonium/ | Silquat ®J2-2B | N/A | Siltech, | 1423679-19-9 |
| diethonium chloride | | | Canada | 57-55-6 |
| PG dimethicone | | | | |
| PEG-8 | Silamine ® | N/A | Siltech, | 182700-78-3 |
| amodimethicone | C-300 | | Canada | |
| Cetyl dimethicone | Silwax ®J1016 | N/A | Siltech, | 191044-49-2 |
| | | | Canada | |
| Organomodified | Silube ® CO | N/A | Siltech, | N/A |
| polysiloxane | Di-10 | | Canada | |
| Organomodified | Silube ® | N/A | Siltech, | 1978330-08-3 |
| polydimethylsiloxane | TMP D219 | | Canada | |

Additional agents

| | | | | |
|---|---|---|---|---|
| Sodium lauryl | SLS | 313.53 | Sigma- | 110863-24-6 |
| sulfate | | | Aldrich, | |
| | | | USA | |
| Molecular sieves | Molecular | N/A | Sigma- | 70955-01-0 |
| | sieves 4 Å, | | Aldrich, | |
| | 1.6 mm beads | | USA | |
| Cobalt chloride | CoCl₂ | 129.84 | Sigma- | 7646-79-9 |
| | | | Aldrich, | |
| | | | USA | |
| Nickel chloride | NiCl₂•6H₂O | 237.69 | Alfa Aesar, | 7791-20-0 |
| hexahydrate | | | USA | |

In the following examples, for conciseness, the material may be referred to by the acronyms indicated in the above table. For instance, AMEO may be used to refer to Dynasylan® AMEO and IPA to refer to isopropyl alcohol.

### Equipment

Confocal laser microscope: Lext 5000 (Olympus, Japan)
Differential Scanning Calorimeter: DSC Q2000 (TA Instruments, USA)
Flat iron: Babyliss® I-Pro 235 Intense protect
Gas chromatographer GC-MS: GCD G1800A (HP, USA)
Hair dryer: Itamar superturbo Parlux 4600 (Parlux®, Italy)

### Ovens:

Drying oven, TZ-150L (Zhengzhou Brother Furnace Co., China)
Muffle oven: 30/1300 (Snol, Germany)
Potentiostat: VSP modular 5 channels potentiostat (BioLogic, France)
Sonicator: DC-1500H (MRC Lab, Israel)
Stirring hot plate: C-MAG HS 7 control (IKA, Germany)
Tensile tester: MTT157 (Dia-Stron, United-Kingdom)
Thermo-rheometer: Thermo Scientific (Germany) Haake Mars III, with a C60/1° spindle, a gap of 0.052 mm
Vortex mixer: Vortex-Genie 2 (Scientific Industries, USA)
Zeta potential: Malvern Zetasizer NanoS (Malvern Instruments, UK)

### Example 1: Preparation of Stock Solutions

### I. PS/DBM Stock

Into a 20 ml cup, 5 g phenyl salicylate (PS) and 5 g dibutyl maleate (DBM) were placed, and the cup was heated using a hair dryer for 30 seconds until complete dissolution. The obtained PS/DBM stock (having a 1:1 weight ratio) was mixed with molecular sieves 4A to reduce any reactions of the stock with environmental humidity, and was maintained for at least 3 days, to ensure the reduction and preferably the elimination of any residual moisture, before further use of the stock.

II. PS/DBM/POSS(EP0409) stock

Into a 20 ml cup, 0.05 g of Glycidyl POSS® Cage Mixture EP0409 were placed, and combined, while stirring at room temperature, with 1.95 g of the PS/DBM stock previously prepared, to obtain a homogeneous solution, containing 2.5 wt. % of POSS® EP0409, 48.75 wt. % of PS and 48.75 wt. % of DBM.

III. PS/DBM/POSS(MA0735) stock

Into a 20 ml cup, 0.1 g of Glycidyl POSS® Cage Mixture MA0735 were placed, and combined, while stirring at room temperature, with 1.9 g of the PS/DBM stock previously prepared, to obtain a homogeneous solution, containing 5 wt. % of POSS® MA0735, 47.5 wt. % of PS and 47.5 wt. % of DBM.

### IV. Shellac Stocks

Into a 20 ml cup, 2 g shellac and 8 g oleyl amine were placed. The cup was placed on a hot plate equipped with a magnetic stirrer, and the mixture was maintained, while stirring, at 160° C. for 40 minutes, whereby a homogenous stock of 20 wt. % shellac was obtained (accordingly referred to as “20% shellac stock”). Another shellac stock was similarly prepared by mixing 3 g shellac with 6 g oleyl amine, containing 33.3 wt. % shellac and accordingly referred to as “33.3% shellac stock”.

### V. Al(O-Sec-Bu 3 Stock

Into a 20 ml cup, 8 g of aluminum tri-sec-butoxide (Al(O-sec-Bu)₃) were combined at room temperature with 2 g of 2-butanol, while stirring for about 5 minutes, to obtain a homogeneous solution.

### VI. PS/DBM/POSS(MA0735)/BPO Stock

Into a 20 ml cup, 0.05 g of Glycidyl POSS® Cage Mixture MA0735 and 0.05 g of benzoyl peroxide (BPO) were placed, and combined at room temperature, while stirring, with 1.9 g of the PS/DBM stock previously prepared, to obtain a homogeneous solution.

### VII. PS/EHS Stock

Into a 20 ml cup, 25 g of phenyl salicylate (PS) and 25 g of 2-ethylhexyl salicylate (EHS) were placed, and the cup contents were heated using a hair dryer for 60 seconds until complete dissolution.

### VIII. Thickener Stocks:

Into a 500 ml cup, equipped with a magnetic stirrer, 0.6 g of Carbopol® Ultrez-20 and 299.4 g of a solution of 40 wt. % urea in water were placed. The contents of the cup were stirred at room temperature for 16 hours until a clear and uniform gel was obtained, and then the pH was adjusted to 8.5 using ammonium hydroxide.

Into a 500 ml cup, maintained under an overhead stirrer set to 800 RPM, 295.5 g of a solution of 40 wt. % urea in water were placed. 3 g Jaguar® HP-105 were gradually added for about 10 min, followed by the addition of 1.5 g of Sepiplus™ S. The contents of the cup were stirred at room temperature for 15 minutes until complete dissolution, and then the pH was adjusted to 8.5 using ammonium hydroxide.

Both thickener stocks (i) and (ii) showed shear thinning behavior, as verified by thermo-rheological analysis, wherein the compositions' viscosities were reduced as the shear rate increased. The thickener stocks were placed in closed containers until further use.

Except for the thickener stocks, the stock solutions prepared in the present example were used, once dried with the molecular sieves, to prepare the oil-in-water emulsions, as described in the following examples.

### Example 2: Oil-In-Water Emulsions Containing a PBM and a WHA

### PBM Compartment

Into a 20 ml vial, 1 g of the PS/DBM stock was placed and combined with 1 g AMEO and 0.5 g of the 20% shellac stock, wherein after the addition of each component, the vial contents were mixed by vortex for about 10 seconds. The obtained mixture was transferred to a 20 ml cup equipped with a magnetic stirrer and stirred for about 80 minutes at room temperature, allowing for the pre-polymerization of a PBM phase.

0.4 g of the pre-polymerized PBM phase was then placed in another 20 ml cup, combined with 0.4 g IPA, and mixed by vortex to obtain a PBM mixture (also termed the PBM compartment).

The ability of a hair styling composition according to the present teachings to be suitably curable, based on the identity of its constituents, their concentrations and relative proportions, can be assessed in vitro at this early stage. A sample of the pre-polymerized PBM phase was allowed to self-level on a microscope glass slide in contact with a hot plate at a temperature of 160° C. Mixtures allowing for the formation of a continuous dry film within 2-3 minutes were considered suitable candidates for further studies and this method enables the screening of numerous compositions ahead of testing on hair fibers.

### Aqueous Compartment:

An aqueous solution containing urea as the water-soluble hygroscopic agent was prepared. In a 100 ml plastic cup, 16 g of a solution of 40 wt. % urea in water having a pH of 10 (separately adjusted using ammonium hydroxide) were placed, 2 g IPA were added, and the obtained aqueous mixture (also termed the aqueous compartment) was mixed by hand for about 10 seconds.

### Oil-In-Water Emulsion:

The contents of the vial containing the PBM mixture were added to the cup containing the aqueous mixture, and vigorously mixed together by hand for about 10 seconds until an emulsion was obtained (“milky” appearance), the emulsion is referred to as PBM-Urea 1 composition, or PU1. A comparative oil-in-water emulsion lacking urea (PBM-No Urea 1 or PNU1) was prepared similarly as PU1, wherein the 16 g of a 40% aqueous urea solution were replaced by 16 g of water having a pH adjusted to 10.

Other oil-in-water emulsions were similarly prepared, containing different components, additives and amounts thereof in each of the two compartments:
PU2, containing POSS® EP0409, was prepared using the PS/DBM/POSS(EP0409) stock, prepared in Example 1, instead of the PS/DBM stock described above;
PU3, containing POSS® MA0735, was prepared using the PS/DBM/POSS(MA0735) stock, prepared in Example 1, instead of the PS/DBM stock described above; and
PU4, containing POSS® MA0735 and benzoyl peroxide, was prepared using the PS/DBM/POSS(MA0735)/BPO stock, prepared in Example 1, instead of the PS/DBM stock described above. The benzoyl peroxide serves to enhance the activation of the acrylate groups in the POSS® MA0735 cross-linker.

These compositions are reported in Table 2, the values reported in the table correspond to the concentration of each component in wt. % by total weight of the emulsion, except for the values in the Pre-polymerized PBM phase, which correspond to the weight percentage of such components in that particular mixture. As the values were rounded up to the closest two digits number, their sum may not exactly add up to 100 wt. %.

TABLE 2

| | Composition | | | | |
|---|---|---|---|---|---|
| Component | PU1 | PU2 | PU3 | PU4 | PNU1 |

Pre-polymerized PBMs phase

| | | | | | |
|---|---|---|---|---|---|
| Phenyl salicylate | 20 | 19.5 | 19 | 19 | 20 |
| Dibutyl maleate | 20 | 19.5 | 19 | 19 | 20 |
| POSS ® EP0409 | | 1 | | | |
| POSS ® MA0735 | | | 2 | 1 | |
| Benzoyl peroxide | | | | 1 | |
| AMEO | 40 | 40 | 40 | 40 | 40 |
| Shellac | 4 | 4 | 4 | 4 | 4 |
| Oleyl amine | 16 | 16 | 16 | 16 | 16 |

PBM compartment

| | | | | | |
|---|---|---|---|---|---|
| Phenyl salicylate | 0.43 | 0.41 | 0.40 | 0.40 | 0.43 |
| Dibutyl maleate | 0.43 | 0.41 | 0.40 | 0.40 | 0.43 |
| POSS ® EP0409 | | 0.02 | | | |
| POSS ® MA0735 | | | 0.04 | 0.02 | |
| Benzoyl peroxide | | | | 0.02 | |
| AMEO | 0.85 | 0.85 | 0.85 | 0.85 | 0.85 |
| Shellac | 0.09 | 0.09 | 0.09 | 0.09 | 0.09 |
| Oleyl amine | 0.34 | 0.34 | 0.34 | 0.34 | 0.34 |
| IPA | 2.13 | 2.13 | 2.13 | 2.13 | 2.13 |

Aqueous compartment

| | | | | | |
|---|---|---|---|---|---|
| Deionized water | 51.06 | 51.06 | 51.06 | 51.06 | 85.11 |
| (pH 10) | | | | | |
| Urea | 34.04 | 34.04 | 34.04 | 34.04 | — |
| IPA | 10.64 | 10.64 | 10.64 | 10.64 | 10.64 |

The presence of aldehydes, and specifically formaldehyde, can be checked in the compositions by gas chromatography-mass spectrometry (GC-MS), according to standard methods (NIOSH 2539 for aldehydes in general and NIOSH 2541 specifically for formaldehyde). A sample of PU1 composition was maintained at 220° C. for 1 hour, allowing at least partial curing of the PBM in addition to the vaporization of the sample volatile constituents, and tested for the presence or formation of formaldehyde. The concentration measured by the detector (Hewlett-Packard GCD, Model G1800A) was found to be below 1 ppm (i.e., less than 0.0001 wt. %), confirming that the hair styling composition is substantially devoid of such SRAs.

### Example 3: Oil-In-Water Emulsions Containing a PBM, a WHA and a Curing Accelerator

Oil-in-water emulsions PU5-PU14 were prepared as described in Example 2, using the 20% or 33.3% shellac stocks and Al(O-sec-Bu)₃ as a curing accelerator, which was added to the PBM phase for the pre-polymerization of the PBM. The pre-polymerization was performed by stirring the PBM phase for about 80 minutes at room temperature.

Comparative oil-in-water emulsions, based on PU5 and PU6, were prepared, in the absence of urea as previously done for PNU1, and are accordingly referred to as PNU5 and PNU6.

These compositions are reported in Tables 3A and 3B, the values reported in the tables correspond to the concentration of each component in wt. % by total weight of the emulsion, except for the values in the Pre-polymerized PBM phase, which correspond to the weight percentage of such components in that particular mixture.

TABLE 3A

Composition

| | | | | |
|---|---|---|---|---|
| Component | PU5 | PU6 | PNU5 | PNU6 |

Pre-polymerized PBM phase

| | | | | |
|---|---|---|---|---|
| Phenyl salicylate | 18 | 17.55 | 18 | 17.55 |
| Dibutyl maleate | 18 | 17.55 | 18 | 17.55 |
| POSS ® EP0409 | | 0.9 | | 0.9 |
| AMEO | 36 | 36 | 36 | 36 |
| Shellac | 3.6 | 3.6 | 3.6 | 3.6 |
| Oleyl amine | 14.4 | 14.4 | 14.4 | 14.4 |
| Al(O-sec-Bu)₃ | 7.2 | 7.2 | 7.2 | 7.2 |
| 2-butanol | 1.8 | 1.8 | 1.8 | 1.8 |

PBM compartment

| | | | | |
|---|---|---|---|---|
| Phenyl salicylate | 0.39 | 0.38 | 0.39 | 0.38 |
| Dibutyl maleate | 0.39 | 0.38 | 0.39 | 0.38 |
| POSS ® EP0409 | | 0.02 | | 0.02 |
| AMEO | 0.77 | 0.77 | 0.77 | 0.77 |
| Shellac | 0.08 | 0.08 | 0.08 | 0.08 |
| Oleyl amine | 0.31 | 0.31 | 0.31 | 0.31 |
| Al(O-sec-Bu)₃ | 0.15 | 0.15 | 0.15 | 0.15 |
| 2-butanol | 0.04 | 0.04 | 0.04 | 0.04 |
| IPA | 2.13 | 2.13 | 2.13 | 2.13 |

Aqueous compartment

| | | | | |
|---|---|---|---|---|
| Deionized water | 51.06 | 51.06 | 85.11 | 85.11 |
| (pH 10) | | | | |
| Urea | 34.04 | 34.04 | — | — |
| IPA | 10.64 | 10.64 | 10.64 | 10.64 |

TABLE 3B

| | Composition | | | | | | | |
|---|---|---|---|---|---|---|---|---|
| Component | PU7 | PU8 | PU9 | PU10 | PU11 | PU12 | PU13 | [symbol] |

Pre-polymerized PBM phase

| | | | | | | | | |
|---|---|---|---|---|---|---|---|---|
| Phenyl | 17.37 | 19.1 | 14 | 17.75 | 19.1 | 35.7 | 36.25 | [symbol] |
| salicylate | | | | | | | | |
| Dibutyl | 17.37 | 19.1 | 14 | 17.75 | 19.1 | 35.7 | 36.25 | |
| maleate | | | | | | | | [symbol] |
| 2- | | | | | | | | |
| ethylhexyl | | | | | | | | |
| salicylate | | | | | | | | |
| POSS ® | 0.89 | 1 | | 0.9 | 1 | | | |
| EP0409 | | | | | | | | |
| AMEO | 34.75 | 39.2 | 58 | 36.4 | 39.2 | | | |
| Shellac | 3.56 | 3.9 | 3 | 3.6 | 3.9 | 7.2 | 7.5 | |
| Oleyl amine | 14.26 | 7.85 | 5 | 14.5 | 7.85 | 14.3 | 15 | |
| Al(O-sec- | 7.13 | 7.85 | 5 | 7.3 | 7.85 | 5.7 | 4 | |
| Bu)₃ | | | | | | | | |
| 2-butanol | 1.78 | 2 | 1 | 1.8 | 2 | 1.4 | 1 | |
| Silwax ® | 1 | | | | | | | |
| B116 or | | | | | | | | |
| H416 | | | | | | | | |

PBM compartment

| | | | | | | | |
|---|---|---|---|---|---|---|---|
| Phenyl | 0.37 | 0.41 | 0.35 | 0.43 | 0.47 | 0.87 | 0.88 |
| salicylate | | | | | | | |
| Dibutyl | 0.37 | 0.41 | 0.35 | 0.43 | 0.47 | 0.87 | 0.88 |
| maleate | | | | | | | |
| 2- | | | | | | | |
| ethylhexyl | | | | | | | |
| salicylate | | | | | | | |
| POSS ® | 0.02 | 0.02 | | 0.02 | 0.02 | | |
| EP0409 | | | | | | | |
| AMEO | 0.77 | 0.83 | 1.41 | 0.89 | 0.95 | | |
| Shellac | 0.08 | 0.08 | 0.06 | 0.09 | 0.1 | 0.17 | 0.19 |
| Oleyl amine | 0.31 | 0.17 | 0.12 | 0.35 | 0.19 | 0.35 | 0.37 |
| Al(O-sec- | 0.15 | 0.17 | 0.11 | 0.18 | 0.19 | 0.14 | 0.10 |
| Bu)₃ | | | | | | | |
| 2-butanol | 0.04 | 0.04 | 0.03 | 0.04 | 0.05 | 0.04 | 0.02 |
| Silwax | 0.02 | | | | | | |
| B116 or | | | | | | | |
| H416 | | | | | | | |
| IPA | 2.13 | 2.13 | | | | | |

Aqueous compartment

| | | | | | | | | |
|---|---|---|---|---|---|---|---|---|
| Deionized | 51.06 | 51.06 | 58.54 | 58.55 | 58.54 | 58.54 | 58.54 | [symbol] |
| water | | | | | | | | |
| (pH 10) | | | | | | | | |
| Urea | 34.04 | 34.04 | 39.02 | 39.02 | 39.02 | 39.02 | 39.02 | [symbol] |
| IPA | 10.64 | 10.64 | | | | | | |

[symbol]

indicates data missing or illegible when filed

Noticeably, compositions PU12, PU13 and PU14 lack a dedicated cross-linker (such as AMEO, contained in the other compositions presented in the above-tables), and the curing accelerator Al(O-sec-Bu)₃ is believed to serve also as a cross-linker in these compositions.

Compositions having a relatively low amounts of cross-linkers or curing accelerators when also acting as a cross-linker, are believed to form polymers behaving in a thermoplastic manner. In order to ascertain the thermoplasticity of the compositions, two drops of each one of PU12, PU13 and PU14 (about 0.05 g) were placed, using a pipette, on a glass slide, and spread by pressing a second slide to the first, whereby a thin layer having an even thickness was formed. The second slides were removed and the glass slides bearing each of the composition layer were placed on a hot plate, heated to a temperature of 160° C., for a period of 5 min, whereby these compositions became tacky to the touch. The glass slides were then removed from the hot plate, allowing the compositions to cool down to room temperature, which resulted in their solidifying. The process was repeated by heating the glass slides to a temperature of 60-70° C., whereby the compositions became tacky again, and re-solidified upon their cooling down once removed from the hot plate again. This behavior is consistent with thermoplastic compositions and is believed to indicate the formation of low cross-linking density polymeric networks.

The preparation of compositions PU12 and PU13 was repeated, wherein the pH of the deionized water in the aqueous compartment was varied between 7.5 and 10, to assess which pH may provide optimal respective charging of the hair fibers and emulsion droplets. For that purpose, the aqueous compartment was prepared as described in Example 2, with the difference that urea was combined with water of neutral pH, and the obtained solution was maintained overnight at room temperature to allow the pH to reach equilibrium. Ammonium hydroxide was then added to adjust the pH as desired (to pH values of 7.5, 8.0, 8.5, 9.0 and 10.0).

Additional compositions were prepared, the PBM phase undergoing an accelerated pre-polymerization by heating the tested mixtures in an oil bath to a temperature of 140-150° C. for about 20 minutes. The progress of the pre-polymerization was monitored by periodically checking the viscosity of the mixtures, wherein about 2 g were sampled and analyzed in a thermo-rheometer at a temperature of 25° C. and a shear rate of about 200 sec⁻¹, until the PBM phase reached a viscosity of about 330 mPa·s. At this viscosity, the PBM mixtures were considered still sufficiently un-polymerized to enable their later penetration in the hair fibers while being sufficiently pre-polymerized to accelerate later straightening once delivered within the hairs. These compositions are reported in Table 3C, as previously described.

TABLE 3C

Composition

| | | |
|---|---|---|
| Component | PU15 | PU16 |

Pre-polymerized PBM phase

| | | |
|---|---|---|
| Phenyl salicylate | 31.5 | 34.5 |
| 2-ethylhexyl salicylate | 31.5 | 34.5 |
| Shellac | 6.5 | 7.1 |
| Oleyl amine | 13.1 | 14.4 |
| α-linolenic acid | 4.4 | |
| Al(O-sec-Bu)₃ | 10.4 | 7.6 |
| 2-butanol | 2.6 | 1.9 |

PBM compartment

| | | |
|---|---|---|
| Phenyl salicylate | 0.8 | 0.85 |
| 2-ethylhexylsalicylate | 0.8 | 0.85 |
| Shellac | 0.2 | 0.2 |
| Oleyl amine | 0.3 | 0.35 |
| α-linolenic acid | 0.1 | |
| Al(O-sec-Bu)₃ | 0.2 | 0.2 |
| 2-butanol | 0.1 | 0.05 |

Aqueous compartment

| | | |
|---|---|---|
| Deionized water | 58.5 | 58.5 |
| (pH 10) | | |
| Urea | 39 | 39 |

### Example 4: Hair Charging Dependency on the PBM-WHA Compositions' pH1

The effect of the pH of a PBM-WHA composition on the zeta potential was tested. Samples of composition PU13 at varying pH of 7, 8 and 9 were prepared as described in Example 3, and the zeta potential of each sample was measured.

Table 4 presents the measured zeta potential, in millivolts (mV) of composition PU13 ((PU13) at the various pH values. Also presented in the table are zeta potential values of virgin hair (ξh), as known from the literature, as well as an absolute value of the zeta potential difference between the composition and the hair (Δξ_{PU13-h}).

TABLE 4

| | Zeta | Zeta | Zeta |
|---|---|---|---|
| | potential | potential | potential |
| | PU13 | virgin hair | difference |
| pH | (ζ_{PU13}) | (ζₕ) | (Δζ_{PU13-h}) |
| 7 | 10.05 | −50 | 60.05 |
| 8 | 3.55 | −55 | 58.55 |
| 9 | −17.9 | −62 | 44.1 |

As can be seen from the above table, all pH values tested were found adequate to promote sufficient zeta potential difference between the styling composition and virgin hair fibers, which will allow the composition to be driven towards the hair. As it is known that bleached hair has more negative binding sites than virgin hair, their zeta potential is expected to be even more negative as compared to virgin hair, so that the delta zeta potential they would display with the present composition would be further increased. Hence, a pH at least in the range of 7.0 to 9.0 is expected to enable charging adapted to drive PU13 to either virgin or bleached hair.

### Example 5: Hair Straightening Using PBM-WHA Compositions

The hair tufts used for testing the straightening ability of the present compositions, containing at least a PBM combined (or not) with a WHA, were curly black hair of Brazilian origin (approximately 30 cm long). Each tuft was glued together at one tip with epoxy glue, and weighted approximately 0.6-1.3 g, including the glued tip. The hair tufts that were used were either virgin (i.e., without any previous treatment), bleached or colored.

Bleaching was performed according to the instructions in the bleaching product package, using either:
a combination of blue anti-orange bleaching powder (“Blu Bleach decolor”, by Elgon, Italy) and a 9% oxygen cream (by Afrodita Cosmetics, Israel), at a 1:2 w/w ratio;
a combination of blue anti-orange bleaching powder (“Blu Bleach decolor”, by Elgon, Italy) and a 12% oxygen cream (Professionnel Oxydant Cream by L'Oréal, France), at a 1:2 w/w ratio; or
a combination of a 4 wt. % ammonium hydroxide solution (in order to emulate a lower ammonium concentration product) and a 9% oxygen cream (by Afrodita Cosmetics, Israel), at a 1:2 w/w ratio.

Coloring was performed using “Kolston naturals” color cream (by Wella, Switzerland), according to the manufacturer instructions.

The curly hair tufts (either virgin, bleached or colored) were washed at 38-40° C. with tap water containing 5% sodium lauryl sulfate to remove any materials adhered to the hair (e.g., dirt or oils), rinsed with excess tap water and dried by either blow drying or by being hanged at room temperature for at least 1 hour, whereby the hair tufts regained their native shapes.

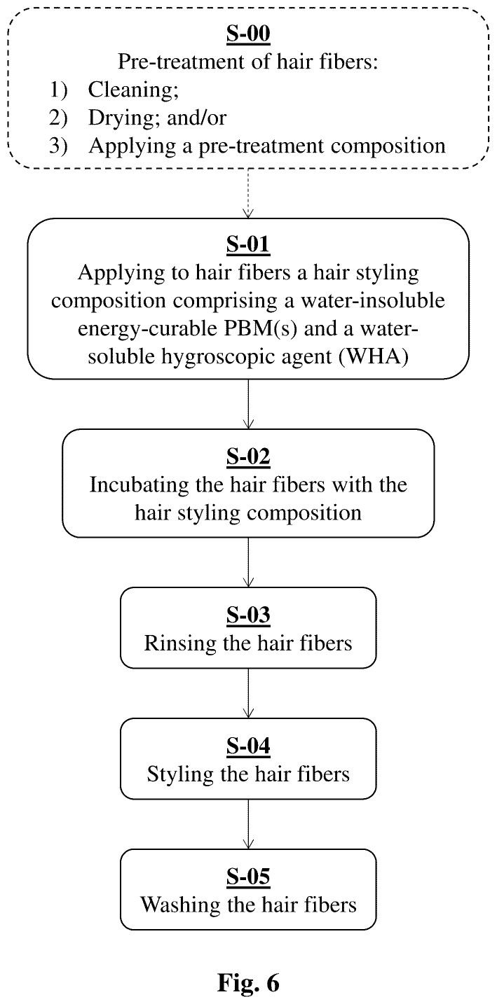

The basic treatment and straightening procedures which were applied to the clean hair samples are schematically depicted in FIG. 6, which shows a simplified diagram of the different steps, which are further described below. While for simplicity, in the present examples the compositions or methods can be referred to as “straightening”, a term usually describing a “complete flattening” of the hair fibers, this term is intended to encompass any significant shape modification, wherein the hair is relaxed to a form less wavy than native shape.

### Procedure:

Application of the composition (as depicted in step S-01 of FIG. 6): the hair tufts were dipped in a 100 ml plastic cup containing about 15-20 g of a hair styling composition (e.g., oil-in-water emulsion), such as prepared in Examples 2-3.

Incubation of the composition (as depicted in step S-02 of FIG. 6): the cups containing the hair tufts samples dipped in the various compositions were maintained, unless otherwise stated, for 1 hour at room temperature, so as to enable at least partial penetration of inter alia the PBM and WHA within the hair fibers.

Rinsing of the hair fibers (as depicted in step S-03 of FIG. 6): the hair tufts so treated were thoroughly rinsed to eliminate excess composition from the surface of the hair fibers. Unless otherwise stated, hair fibers were rinsed with tap water at a temperature of about 38-40° C. for 10-20 seconds, and then dried using a hair dryer for 2-3 minutes.

Styling of the hair fibers (as depicted in step S-04 of FIG. 6): the rinsed and dried treated hair tufts were then straightened using a flat iron, at a temperature of 220° C. for about 2-3 minutes (about 30-50 passes), depending on the tuft length, until the tufts were completely dried and in the desired modified shape. This step allows at least partial curing of the PBM(s).

Washing of the hair fibers (as depicted in step S-05 of FIG. 6): the styled hair tufts were then washed by finger rubbing for about 30 seconds a standard shampoo on the hair fibers to ensure full tip to tip coverage and intimate contact with the hair. The shampooed hair tufts were then rinsed with tap water at a temperature of about 38-40° C., similarly “massaged” with a hair conditioner for about 30 seconds and rinsed again with tap water at the same temperature. The rinsed hair tufts were then fully dried using a hair dryer. Unless otherwise stated, the standard shampoo was Shea Natural Keratin Shampoo by Saryna Key, Israel, and the hair conditioner was Pro Collection, Biotin+Repair 7, by TRESemmd, Unilever, USA.

As readily appreciated, steps of the above-described procedure are exemplary and may be performed under different conditions. The hair styling procedure may also include additional steps. One such optional step S-00 (accordingly marked in a dashed contour in FIG. 6) includes the pre-treatment of the hair fibers before applying the composition in step 1, wherein the hair fibers may be washed and/or residual water may be removed from the hair tufts using, e.g., a flat iron, passed a few times over the tufts at an elevated temperature, such as 200° C.; and/or a pre-treatment composition (e.g., an oil) may be applied.

Another optional step includes further curing of the at least partially cured polymerizable styling composition, once within the hair fibers. The step may include exposing the hair tufts, straightened and dried following the styling step 4 or the washing step 5, to further heating, using a hair dryer, to accelerate the further curing of the PBMs having already at least partially polymerized in the hair fibers in earlier steps. The hair samples may, for instance, be maintained on a brush, having a hair dryer rapidly moved at a short distance over the tufts about 15 times, blowing air at a temperature of 150-220° C., so that the hair fibers perceive an elevated temperature of at most 220° C. for a few seconds.

Alternatively, or additionally, a curing composition comprising excess amount of a curing facilitator may be briefly applied, for instance by rinsing with a dedicated solution containing such materials. Similarly, prior to styling of the hair fibers (S-04), the hair can be treated with a formulation protecting the hair from damages that may result from the temperature applied during styling (as shall be described hereinafter). Such a heat-protective formulation can contain or consist of oils having a relatively high smoking point at a temperature above the one applied for styling. Silicone oils can be used for this purpose. Alternatively, the styling composition may include an agent providing for such heat protection, for illustration a suitable lubricant (e.g., a silicone oil) can be added to the PBM, the oil being non-miscible with the PBMs so as to form therewith an oil-in-oil emulsion allowing it to leach out to protect the PBMs during styling, so they remain sufficiently active to polymerize. Typically, a suitable non-miscible oil would have a density lower than the density of the PBM, so as to migrate out when the emulsion dissociates under heat. Such a composition is illustrated by the two versions of PU7 (each using a different type of silicone oil), as prepared in Example 3.

### Example 6: Durability of the Hair Straightening

The hair tufts, treated with the compositions of the present invention as described in Example 5, were subjected to a series of washings starting 48 hours after the washing step 5. In each washing cycle, the hair tufts were washed with a shampoo and a conditioner, as described in washing step 5 of Example 5. The washing cycles were performed up to five times a day, typically within at least one hour one of the other.

The number of washes after which the hair tufts remained “straightened”, including any type of modified shape originally obtained at the end of the straightening procedure of Example 5, is indicative of the durability of the hair styling provided by the present compositions and method. This number can also be referred to as the “wash resistance” afforded by a particular composition under the conditions it was applied and tested. Wash resistance can be visually assessed by trained operators in a qualitative manner, the result provided indicating the number of washing cycles following which changes in shape become visibly detectable. Alternatively, wash resistance can be quantified, for instance by measuring the length of the hair samples after styling treatment and after any desired amount of washing cycles, and/or by counting the number of deviations from straight hair (e.g., peaks and dips) in a representative number of fibers. Length can be measured by placing the hair fiber along a ruler, without stretching or pulling the hair fiber. The number of “twists” in the hair fiber can be provided by counting the number of amplitudes (minimum and maximum) visible on the fiber. The number of twists can be normalized to the hair length and the straightness efficiency can be calculated by dividing the normalized number of twists after treatment being considered by the normalized number of twists before such treatment (the reference). Straightness efficiency can be expressed as a percentage of the reference. The hair fibers are “wash resistant” as long as the measurements (e.g., length, number of twists, or straightness efficiency, before washing and at the washing cycle being considered are similar (e.g., within 10% or less one from the other) or as long as trained operators are unable to detect visible changes. Similarly, such methods can be used to assess the effect of the hair styling composition.

Tables 5A and 5B present the wash resistance of the compositions of Examples 2-3, as applied to hair tufts treated and straightened as described in Example 5, the results being qualitatively assessed by trained operators. Table 5A presents the maximal wash resistance tested on virgin hair, treated by the various compositions (prepared at various pH). The value reported for PU7 applies to both versions of the composition, the one including Silwax® B116 and the one including Silwax® H416.

TABLE 5A

| | |
|---|---|
| PBM ± WHA | Virgin hair |

| emulsion | Washes | pH |
|---|---|---|
| PU1 | ≥90 | 10 |
| PU2 | ≥13 | 10 |
| PU3 | ≥11 | 10 |
| PU4 | 8 | 10 |
| PU5 | 37 | 10 |
| PU6 | 27 | 10 |
| PU7 | 6 | 10 |
| PU8 | 50 | 10 |
| PU9 | 21 | 10 |
| PU10 | 10 | 10 |
| PU11 | 51 | 10 |
| PU12 | 8 | 5 |
| | 7 | 7 |
| | 28 | 10 |
| PU13 | 12 | 5 |
| | 14 | 7 |
| | 14 | 7.5 |
| | 14 | 8 |
| | 14 | 8.5 |
| | 14 | 9 |
| | 12 | 10 |
| PNU1 | 4 | 10 |
| PNU5 | 5 | 10 |
| PNU6 | 5 | 10 |

As can be seen from the above-table, all PU1-PU13 compositions containing a water-soluble hygroscopic agent (the WHA being urea in the present case), provided for a wash resistance of six or more cycles, reaching a wash resistance of over 90 washes, supporting at least partial penetration of the PBMs within the hair fibers and their polymerization therein. These conclusions concerning penetration of at least part of the hair styling compositions within the hair fibers are further supported by the FIB-SEM analysis previously reported with reference to FIGS. 2A, 2B and 3B.

In the table, the symbol ≥before a reported number of washing cycles indicates that the experiment was interrupted at this stage, so that the wash resistance afforded by these compositions may be greater, or even significantly greater, than the reported value.

By comparison, the hair fibers that were treated by the compositions lacking urea (namely, PNU1, PNU5 and PNU6) provided for a hair straightening which lasted for 5 washes or less. Therefore, in the present example, the presence of the WHA extended the wash resistance to be at least 5-fold the wash resistance of a similar composition devoid of the WHA In the case of PU1 as compared to PNU1, the impact of the WHA is even more dramatic, the increased wash resistance being of more than 20-fold. As previously explained, these results are highly surprising in view of the relatively high water-solubility of the WHA which were expected to wash away at a very early stage. It is believed that the WHA having penetrated within the hair fibers forms therein resilient water-capturing bodies, and/or interactions with the cured polymer and/or with the hair constituents.

The efficacy of some of the hair styling compositions was also tested on previously treated hair, namely, bleached or colored, and the wash resistance results are reported in Table 5B as the weighted average of repeat experiments.

TABLE 5B

| PBM ± | Bleached hair | | | | | | | |
|---|---|---|---|---|---|---|---|---|
| WHA | Treatment A | | Treatment B | | Treatment C | | Colored hair | |
| emulsion | Washes | pH | Washes | pH | Washes | pH | Washes | pH |
| PU8 | 15 | 10 | 23 | 10 | | | | |
| PU11 | 16 | 10 | 10 | 10 | 15 | 10 | 6 | 7 |
| | | | | | | | 14 | 10 |
| PU12 | 7 | 5 | | | | | | |
| | 8 | 7 | | | | | | |
| PU13 | 9 | 7 | | | | | | |
| | 13 | 7.5 | | | | | | |
| | 11 | 8 | | | | | | |
| | 13 | 8.5 | | | | | | |
| | 9 | 9 | | | | | | |

Hair fibers previously treated by bleaching or coloring resisted a weighted average of at least 7 washes, reaching a weighted average of as many as 23 washing cycles, indicating that the present hair styling compositions are also effective on damaged hair.

As can be seen, while a pH of 7-9 was found to be adequate to allow a satisfactory zeta potential difference as demonstrated in Example 4, the tested compositions showed efficacy and durability for both virgin and damaged hair (bleached or colored) over an even broader pH range of 5-10. Understandingly, factors other than the pH of the hair styling compositions may influence their efficacy as presently assessed by the resistance of the styling effect to repeated washes.

In a second series of experiments, the hair styling procedure was modified to include, following the application of the hair styling composition, its incubation with the hair and its rinsing away, a step intending to protect the hair fibers from the subsequent heat-induced straightening and/or to ensure they remain individually separated. For this purpose, a fluorinated silicone lubricant (Fluorosil® J15, by Siltech) was applied on the dried hair obtained at the end of step 3, before performing the ironing of step 4 as described. This was performed on hair fibers treated in step 2 with PU6. Interestingly, while hair samples treated by the unmodified protocol achieved a wash resistance of 25 cycles, the hair samples which benefited from the additional pre-conditioning of the hair samples with the lubricant succeeded to maintain the styled shape for up to 39 wash cycles. These results support the beneficial effect of such a step, if further included in the hair styling method.

### Example 7: Oil Pre-Treatment of Hair

While styling of hair fibers as described in Example 5 yielded satisfactory results (as evidenced by the afforded wash resistance described in Example 6), the procedure can be modified by including an oil pre-treatment step.

### Screening for Suitable Oils

### Lack of Penetration:

The penetration of an oil candidate to the hair can be assessed as follows: a group of hair fibers, untreated by the compositions of the present invention, is weighed and placed in a cup containing the tested oil for a sufficient amount of time to allow possible penetration into the hair. After that time, the hair fibers are removed from the oil, wiped clean, and weighed again. Any increase in hair weight compared to their weight before their immersion in the oil can be attributed to the oil having penetrated into the fibers. Oils that cause a weight gain of less than 5% are considered suitable for their further screening as pre-treatment oils.

### Lack of Polymerization Inhibition:

An inhibitory activity by an oil candidate can be assessed by applying a thin layer of the tested oil on a glass slide, followed by the application of a layer of a curable styling composition according to the present teachings which is to be tested for compatibility with the proposed pre-treating oil. The glass slide is then subjected to application of energy at appropriate temperature and for a sufficient amount of time to induce full curing of the styling composition (e.g., placing on a hot plate or in an oven). The slide with the cured layer of styling composition is allowed to cool. The cured layer is then peeled away from the slide and wiped clean of any residual oil that was applied beneath it on the slide. If the side of the cured composition that was in contact with the oil remains tacky, this is indicative that the curing was not complete, in which case the tested oil is believed to have an inhibitory effect on proper polymerization of the hair styling composition. Conversely, if the sides in former contact with the oil and with the air are similarly non-tacky, then the tested oil is considered suitable for further screening as a pre-treatment oil.

### Lack of Miscibility:

The miscibility of an oil candidate with the styling composition to be applied thereon was tested as follows. 0.05 g of the tested oil were added to 0.95 g of the hair styling composition under study, thoroughly mixed by vortex for 10 seconds and the mixture was allowed to separate into distinct phases. Oils that were found immiscible with the styling composition were deemed suitable as pre-treatment oils for the later application of said composition.

Silquat®J2-2B, Silamine® C-300, Silwax®J1016, Silube® CO Di-10, and Silube® TMP D219 were found to lack miscibility with the PU5 composition, Silube® TMP D219 being additionally non-miscible with PU6.

Pre-Treatment with the Selected Oils

Virgin hair fibers were pre-treated with each one of the selected oils prior to the first step of the procedure described in Example 5 of applying the hair-styling composition.

Into 100 ml plastic cups, 20 g IPA were mixed by hand with 0.2 gr of each of the pre-treatment oils. Hair tufts, previously washed with sodium lauryl sulfate, rinsed and dried as described in Example 5, were dipped in the various oil/IPA mixtures and maintained for 5 minutes at room temperature. The hair tufts were then rinsed with tap water and blow dried for a few minutes until they were completely dried.

Each one of the pre-treated tufts were then styled with the PU5 or PU6 compositions prepared in Example 3, according to the procedure described in Example 5, and the durability of the styling treatment was tested as described in Example 6.

The pre-treatment oils and styling compositions used in combination are presented in Table 5, together with the wash resistance results.

TABLE 5

| Pre-treatment | | |
|---|---|---|
| oils (commercial | Styling | Max. wash |
| name) | composition | resistance |
| Silquat ®J2-2B | PU5 | 11 |
| Silamine ® C-300 | | 40 |
| Silwax ®J1016 | | 13 |
| Silube ® CO Di-10 | | 40 |
| Silube ® TMP D219 | | >35 |
| Silube ® TMP D219 | PU6 | >25 |

The oil pre-treatments tested in the present study allowed the styling activity of the compositions tested therewith, while improving the feel and combability for all the tested hair tufts, as assessed by trained operators. Hair fibers styled with PU6 were analyzed by FIB-SEM microscopy in order to assess the effect of Silube® TMP D219 on the transient coating that may initially form on the outer surface of treated hair fibers. While after only two washes hair fibers which were not pre-treated with the oil displayed a transient coating having a thickness of up to 1 μm (later washed away), hair fibers pre-treated with the oil after a same number of washes did not display a detectable transient coating on their outer surfaces.

### Example 8: Detection of Urea in Hair Fibers

As readily appreciated by a skilled person, the presence of components as herein disclosed for the present composition can be detected in the composition by any standard method adapted for the identification of the component of interest (e.g., the PBMs, the WHAs, the curing facilitators etc.) using any suitable equipment adapted for such analysis. The present example, however, is concerned by the detection of components of the composition having successfully penetrated into the hair fibers following the application of the hair styling composition. In particular, as the present compositions contain PBMs and WHAs, the present study specifically relates to the detection of urea (i.e. a WHA).

Detection of urea in extracts obtained from hair samples treated with the compositions of the present invention was performed by Electrochemical Impedimetric Spectroscopy (EIS), using an interdigitated gold electrode. The interdigitated electrode was coated with nickel cobalt oxide (NiCo₂O₄), an electro-catalyst enhancing the electro-oxidation of urea, resulting in the formation of conductive species and an increase of the solution conductivity. The use of such technology allows for the detection of low amounts of urea.

Synthesis of NiCo₂O₄

Nickel cobalt oxide was synthesized from a growth solution, prepared by placing in a 150 ml glass cup: 1.300 g of cobalt chloride, 1.185 g of nickel chloride hexahydrate, 2.000 g of urea and 75 mL of distilled water, and stirring for 30 minutes using a magnetic stirrer until a homogeneous solution was obtained. The cup containing the growth solution was sealed with aluminum foil and kept in a preheated drying oven at a temperature of 95° C. for 5 hours. The formed precipitate was filtered out of the growth solution, washed with distilled water and maintained at room temperature overnight, to allow its drying. The obtained dried powder was then calcined in a muffle oven at a temperature of 500° C. for 3 hours.

Fabrication of a NiCo₂O₄-Coated Interdigitated Electrode

4 mg of the calcined NiCo₂O₄ powder and 1 ml of isopropyl alcohol were placed in a 20 ml glass vial and sonicated for 15 minutes. 0.5 ml of 5% Nafion™ solution was added and mixed by a vortex mixer to obtain a suspension.

An interdigitated gold electrode (by Eltek, Israel) was coated by the NiCo₂O₄-Nafion™ solution by drop-casting as follows: four drops (about 100 μl) of the solution were placed on the interdigitated part of the electrode, and the electrode was maintained at room temperature for 3 hours, to allow evaporation of the liquids, whereby a dry coating of NiCo₂O₄ and Nafion™ was formed on the electrode surface.

### Preparation of Extract Samples of Hair Fibers

Two hair tufts (each tuft from a different source) were treated by composition PU12 according to Example 5 and washed 12 times with a cleaning product devoid of urea, to remove any external trace of this material.

Ten hair fibers of each tuft were put in two 20 ml vials, each containing 2 g distilled water, and the vials were placed in an oven at a temperature of 70° C., while being constantly shaken at 200 RPM. After 12 hours, the vials were taken out of the oven, and allowed to cool to room temperature. Each one of the hair samples was filtered out, and the obtained extracts were transferred for impedance analysis.

A reference extract sample was similarly prepared, using hair fibers that have not been treated with a PBM-urea composition, but merely washed twelve times with tap water containing 5% sodium lauryl sulfate and rinsed.

### Measurement of Electrochemical Impedance

Urea detection in the extract solutions was performed based on the electrochemical impedance measurements of the solutions, according to the following oxidation reaction (catalyzed by the NiCo₂O₄ electro-catalyst coating the electrode), whereby conductive ions are formed:

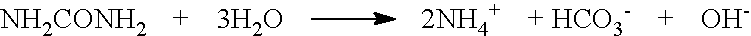

The generated ions contribute to the impedance change in the solution, which is proportional to their concentration, and is thus indicative of the urea concentration in a tested solution,

Electrochemical impedance measurements were carried out at room temperature using a Frequency Response Analysis (FRA)-equipped potentiostat (in combination with the EC-Lab© software) in potentiostatic mode, at a frequency range of 100 kHz to 1 Hz and at constant electric potentials: either at 0 mV or at the urea oxidation voltage of 850 mV (as previously determined). A conventional assembly of two-electrodes setup was used, wherein the working electrode cable was connected to one side of the NiCo₂O₄-coated interdigitated electrode, and the counter electrode and reference electrode were together attached to the other side of the interdigitated electrode. The NiCo₂O₄-coated interdigitated electrode was manually cleaned before each scan by washing with water, followed by drying by a compressed air blower.

Impedance measurement runs were carried out for each of the two tested samples, wherein a potentiostatic step was first carried out, in which each of the selected potentials was exerted on the electrode for 10 seconds (a potential of 850 mV was applied to initiate the oxidation reaction, and a potential of 0 mV was applied as reference).

The resistance value (R) was calculated by means of Zfit data processing for each extract solution, and then converted to conductivity (C=1/R). The calculated conductivity values were used to determine the relative conductivity change (ΔC/C), wherein ΔC is the difference between the conductivity at 850 mV and the conductivity at 0 mV, divided by the conductivity at 0 mV. The conductivity change was further divided by the sample mass (m), to provide a normalized value. The detection limit of this method was found to be about 0.001 mg⁻¹, determined according to the untreated reference sample.

Impedance of the two extract samples was measured, and (ΔC/C)/m values were calculated and found to be on average of 0.024 mg⁻¹, indicating the presence of urea in the extracts. Since extractions of hair fiber contents was performed after 12 washing cycles, it is believed that the urea detected in the extracts originated from the composition PU12 which actually penetrated the hair fibers.

A comparative experiment was performed, wherein 10 hair fibers were dipped in 20 ml of a 40% urea solution for 1 hour. The fibers were then removed from the urea solution and washed 12 times, as previously described. An extract sample was obtained from the washed fibers and impedance measurements were performed, as described above.

The (ΔC/C)/m value obtained after 12 washes of hair fibers exposed to a high concentration of urea was 0.002 mg⁻¹, close to the baseline of 0.001 mg⁻¹ found in absence of urea, indicating that most of the urea that was present within or outside the hair fibers was washed out. This value is lower than those obtained with hair treated by composition PU12, suggesting that the hair styling composition according to the present teachings not only penetrated into the hair fibers, as supported by the presence of urea, but also restricted or reduced the urea washing out of the hair (which, as shown in the comparative experiment, occurs more freely in absence of any polymeric styling composition).

### Example 9: Restyling of Hair Treated with Compositions Containing PBM-WHA

Hair samples treated with compositions such as prepared in Examples 2-3, which display a wash resistance as tested in Example 6 deemed sufficient (e.g., confirming the formation of a PBP within the fibers by resisting at least 10 wash cycles or any other set number of cycles) may undergo a restyling treatment, e.g., straightening of the hair fibers, as described in step 4 of Example 5. This heat treatment is expected to sufficiently soften the formed polymer to reshape the hair fibers, in order to restyle the hair fibers. The restyling may be to the same shape as provided by the original styling treatment or to any other second modified shape that can be applied to the fibers. After the application of heat, the hair samples are allowed to cool back to room temperature, allowing the polymer to regain its stiff/unsoftened structure. Hair samples so restyled can be subjected to washing cycles as described in Example 6 to assess the resistance of the reshaped polymer and the ongoing protective effect of the WHA.

### Example 10: De-Styling of Hair Treated with Compositions Containing PBM-WHA

Hair samples treated with compositions such as prepared in Examples 2-3, which display a wash resistance as tested in Example 6 deemed sufficient (e.g., confirming the formation of a PBP within the fibers by resisting at least 10 wash cycles or any other set number of cycles) and still being in a styled (straightened) shape may undergo a destyling treatment allowing the hairs to regain their original (unmodified, e.g., curly) shape.

The styled hair samples can be dipped in a 100 ml plastic cup containing about 15 g of a de-styling liquid being an ammonium solution having a pH of 10.5. The cups are then placed in a digital orbital shaker and shaken for 1 hour at 60° C. The appearance of the hair samples so “de-styled” is compared to the original appearance of the native untreated hair samples to assess the efficacy of the de-styling treatment. The Inventors posit that the de-styling process does not cause the elimination of the synthetic polymer entrapped within the hair fibers, as can be confirmed by the ability to further re-style the hair samples, as previously described.

### Example 11: Differential Scanning Calorimetry (DSC) Study

Keratin hair fibers demonstrate characteristic endothermic peaks in a number of thermal analytical methods, each peak being indicative of chemical changes occurring near the various temperatures. The hair samples treated according to Examples 5 and 6 can be analyzed by DSC to assess the effect of the composition of Examples 2-3 on the physico-chemical properties of the hair fibers and compare them to an untreated reference of a same hair type.

The reference and treated hair samples are cut into small pieces (about 2 mm long) using regular scissors. For each measurement, about 5 mg of hair pieces are placed in a 70 μl platinum DSC crucible. The crucible is kept open during measurements.

The samples are placed in a Differential Scanning Calorimeter, and DSC measurements are carried out. Specifically, the samples are heated to 400° C. at a rate of 10° C./minute under nitrogen, while data acquisition and storage are performed.

The stored data is plotted to obtain DSC curves for each of the hair samples and values of endotherm points are retrieved. If the modified and native hair fibers display at least one essentially similar endotherm temperature, the composition having achieved this modification is deemed innocuous. Endotherm temperatures of two materials or hair fibers can be considered essentially similar if within 4° C., 3° C., 2° C., or 1° C., from one another.

FIG. 5 depicts the results of a DSC study showing how a non-damaging hair styling method, such as proposed by the present invention, may keep the hair unharmed, as opposed to traditional methods. As can be seen in the figure, the curve of a sample of hair fibers treated with a hypothetical innocuous composition of the present invention would be comparable to the curve of untreated, native hair sample, indicating no significant structural changes. The solid line at the bottom of the plot depicts the curve of untreated curly black hair fibers. Two endotherms are observed, at 234.5° C. and 250° C., which are the characteristic temperatures for hair fibers. The first endotherm around 234.5° C. is believed to indicate the melting of α-keratin in the fiber, while the second endotherm around 250° C. is believed to indicate the keratin decomposition and breaking of the di-sulfide bonds.

In contrast, the DSC curves of commercial hair straightening methods (organic and Japanese) actually tested against the untreated reference show substantial changes from the native hair sample curve, indicating structural changes, which are to be expected when using such drastic hair styling methods.

Such measurements can alternatively be obtained from other methods of thermal analysis, such as by thermomechanical analysis (TMA) or dynamic mechanical analysis (DMA).

### Example 12: Mechanical Properties of Hair Fibers

The hair samples treated according to Examples 4 and 5 were analyzed by tensile testing to assess the effect of the compositions of the present invention (such as prepared in Examples 2-3) on mechanical properties of the hair fibers and compared to an untreated reference of a same hair type.

Ten hair fibers were taken from each one of a reference sample and a PU6 treated hair sample, and standardized by maintaining them under the same conditions for three days (e.g., a temperature of 25° C. and 45% RH). The hair fibers were then cut to a length of 30 mm, and their representative cross-section was measured by confocal laser microscopy, taking into account both the largest radius and the smallest radius of typically elliptical hair fibers. The tensile parameters, extension at break, break stress, toughness and elastic modulus, were measured for the examined hair fibers by tensile tester (at 100% extension limit, 20 mm/minute extension rate, 2 g gauge force, 5 g break detection limit and 2000 g maximum force). The average results for the ten fibers of the treated hair sample were compared to those of the reference sample.

Hair fibers straightened with PU6 were found to have an extension at break, a break stress, and a toughness comparable to the untreated hair fibers, the average results being mildly superior to an extent not statistically significant. Only in the elastic modulus, were the samples treated by the present composition found superior to the untreated samples. These results were observed on set of hair fibers obtained from two different sources. For comparison, hair fibers treated by organic straightening (known to be damaging, as shown in the DSC Example) were typically found inferior to the untreated reference, and in turn to the hair fibers treated by the present method. For illustration, while the hair samples straightened by the present method displayed a toughness 10% higher than untreated hairs, hair samples straightened by the organic technique displayed a toughness 30% lower. Similarly, the hair samples straightened by the present method displayed a break stress 6% higher than untreated hairs, while in contrast the hair samples straightened by the organic technique displayed a toughness 12% lower. Regardless of the statistical significance of the present results, it can be said with confidence that the present method and compositions at the very least does not damage the hair fibers and may even improve them.

Though these observations were made on a single sample treated by a present composition and method, they are believed to illustrate a trend applicable to other compositions as well.

Generally, the break stress of the treated hair fibers is expected to be at least 5%, at least 10%, at least 20% or at least 25% greater than the break stress of similar untreated fibers. Furthermore, the treated hair fibers are expected to have a toughness of 95% or more, 100% or more, 105% or more, 110% or more, 115% or more, or 120% or more of similar untreated hair fibers. The elastic modulus of both treated and untreated samples is expected to be at least comparable, as a negative control such as organic hair straightening did not seem to affect this particular parameter.

### Example 13: Thickening of Oil-In-Water Emulsions Containing a Pre-Polymerized PBM and a WHA

16 g of a pre-polymerized PBM phase, as prepared for PU16 described in Example 3, was mixed with 160 g of thickener stock (i) having its pH adjusted to 8.6 by ammonium hydroxide, prepared according to Example 1. The two phases were vigorously mixed together using a formula frother mixer until an emulsion referred to as PU17 was obtained.

This composition, as well as others, similarly prepared, are reported in Table 6, the values reported in the table correspond to the concentration of each component in wt. % by total weight of the emulsion, except for the values in the Pre-polymerized PBM phase, which correspond to the weight percentage of such components in that particular mixture. The pH values of the thickened aqueous compartment, as well as the viscosities of thickened final oil-in-water emulsions (tested using a thermo-rheometer at a temperature of 25° C. and a shear rate of 200 sec⁻¹) are also specified.

TABLE 6

| | Composition | | |
|---|---|---|---|
| Component | PU17 | PU18 | PU19 |

PBM compartment

| | | | |
|---|---|---|---|
| Phenyl salicylate | 3.15 | 0.85 | 1.6 |
| 2-ethylhexyl salicylate | 3.15 | 0.85 | 1.6 |
| Shellac | 0.6 | 0.2 | 0.3 |
| Oleyl amine | 1.3 | 0.35 | 0.7 |
| Al(O-sec-Bu)₃ | 0.7 | 0.2 | 0.4 |
| 2-butanol | 0.2 | 0.05 | 0.1 |

Thickened aqueous compartment

| | | | |
|---|---|---|---|
| Deionized water | 54.4 | 58.4 | 56.3 |
| Urea | 36.3 | 38.9 | 37.5 |
| Jaguar ® HP-105 | | | 1 |
| Sepiplus ™ S | | | 0.5 |
| Carbopol ® Ultrez 20 | 0.2 | 0.2 | |
| polymer | | | |
| pH of thickened | 8.6 | 8.6 | 8.6 |
| aqueous compartment | | | |
| Composition viscosity | 290 | 290 | 660 |
| [mPa · s] | | | |

An additional composition was prepared, referred to as PU20, containing similar components at similar concentrations as PU19, and using a thickened aqueous compartment having a pH of 10. These compositions were then used to style the hair of human volunteers, as described in the following example.

### Example 14: Field Testing of Hair Styling Compositions

The hair styling compositions according to the present teachings were tested on human volunteers having hair exceeding a length of at least 25 cm. The volunteers had wavy to curly hair, their hair being either virgin (i.e., uncolored nor chemically processed in any way), colored with a conventional coloring formulation, or bleached and colored. All volunteers entered the study protocol with clean and dry hairs.

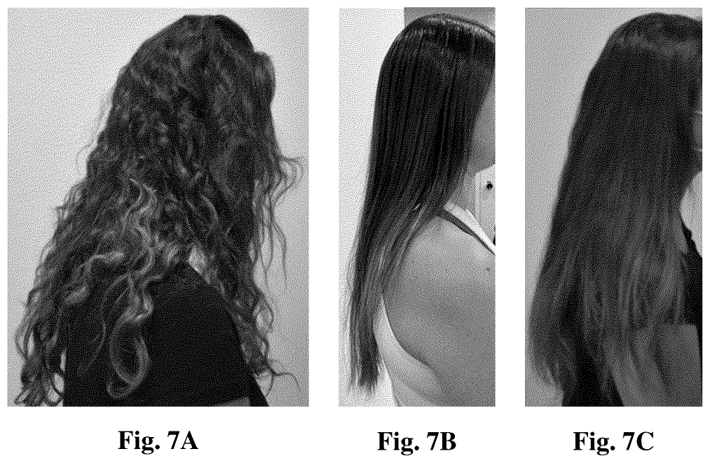

A representative volunteer, having natural untreated hair, is shown as the study proceeds in FIGS. 7A-C. FIG. 7A is a photography of the volunteer's profile, taken before the study as baseline, where her curly hair is clearly visible. Composition PU17 was used for this volunteer, the previously described PBM compartments and thickened aqueous compartment being mixed right before application of the composition to the hair.

The volunteer's hair was treated with the composition in a manner similar to the in vitro procedure described in Example 5, with the following modifications. As the hairs cannot be dipped in a beaker, the thickened composition was applied by a professional hairdresser with a brush on individual groups of hair fibers at a time, until the hair of the entire scalp was coated with the tested hair styling composition. Following its application, the hair styling composition was allowed to remain on the hair fibers for an incubation period of about one hour at room temperature. The volunteer's hairs were then thoroughly rinsed with tap water at a temperature of about 35-40° C. and completely dried using a hair dryer. The hairs of the volunteers were then straightened using a flat iron, at a temperature of 220° C. for about 2-3 minutes (about 30-50 passes) for each wick of hair, until the hairs assumed the desired modified shape. This can be observed in the photography of FIG. 7B taken right after the styling treatment, where the volunteer's hair is completely straightened. At this stage, the hair was defined as “styled”.

From the moment the hair was “styled”, the volunteer refrained from washing her hair for a period of 2 days, after which time, the volunteer washed her hair at regular times once every three or four days with a conventional shampoo and conditioner (Bio Renew Pure Aloe & Avocado Oil shampoo and conditioner, by Herbal Essences). FIG. 7C shows a picture of the same volunteer after 18 washes of her hair (about 2 months since initial treatment), her hair being evidently still straightened, as compared to its original state shown in FIG. 7A. This volunteer reported ongoing substantial straightening 6 months after initial treatment, the area of growing hair near the scalp being excluded, since previously untreated.

Other volunteers, having natural or previously colored/bleached hair, were similarly treated with additional compositions described in Table 6 of Example 13. All compositions on all types of hair provided for a significant initial straightening comparable to the effect shown in FIG. 7B. Noticeably, the styling treatment was efficacious even on bleached hair, often incapable of being straightened by conventional methods when already too damaged to further sustain usually deleterious straightening. The durability of the styling treatment, namely the number of washes after which the hair remained straightened, as well as the time elapsed since the initial treatment with the compositions, are reported in Table 7.

TABLE 7

| | Treating | Number of |
|---|---|---|
| Hair type | composition | washes |
| Natural | PU17 | 18 |
| Colored | PU19 | 25 |
| Bleached and colored | PU20 | 15 |

The volunteers were satisfied by the initial treatment with the hair styling compositions, perceived as relatively odor-less, less irritating, not altering hair color, nor hair health, and providing for a natural appearance. It is stressed that as the study is still ongoing with some volunteers, the efficacy of the present compositions to maintain a desired hair style may extend beyond the values currently reported. All volunteers expressed satisfaction with the results after at least 2 months, their hairs maintaining a straightened shape deemed by them relatively natural. Other parameters appreciated by the volunteers included the combability, shine, feel and reduced frizz of the treated hair.

It should be noted that a common side-effect of standard hair-styling treatments (e.g., organic or keratin straightening) is the lightening of the hair color. Therefore, the fact that the present compositions avoid this side effect, allowing hair styling without modifying the color of the treated hair whether dyed or natural, is deemed advantageous.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Although the present disclosure has been described with respect to various specific embodiments presented thereof for the sake of illustration only, such specifically disclosed embodiments should not be considered limiting. Many other alternatives, modifications and variations of such embodiments will occur to those skilled in the art based upon Applicant's disclosure herein. Accordingly, it is intended to embrace all such alternatives, modifications and variations and to be bound only by the spirit and scope of the disclosure and any change which come within their meaning and range of equivalency.

In the description and claims of the present disclosure, each of the verbs “comprise”, “include” and “have”, and conjugates thereof, are used to indicate that the object or objects of the verb are not necessarily a complete listing of features, members, steps, components, elements or parts of the subject or subjects of the verb. Yet, it is contemplated that the compositions of the present teachings also consist essentially of, or consist of, the recited components, and that the methods of the present teachings also consist essentially of, or consist of, the recited process steps.

As used herein, the singular form “a”, “an” and “the” include plural references and mean “at least one” or “one or more” unless the context clearly dictates otherwise. At least one of A and B is intended to mean either A or B, and may mean, in some embodiments, A and B.

Unless otherwise stated, the use of the expression “and/or” between the last two members of a list of options for selection indicates that a selection of one or more of the listed options is appropriate and may be made.

Unless otherwise stated, when the outer bounds of a range with respect to a feature of an embodiment of the present technology are noted in the disclosure, it should be understood that in the embodiment, the possible values of the feature may include the noted outer bounds as well as values in between the noted outer bounds.

As used herein, unless otherwise stated, adjectives such as “substantially”, “approximately” and “about” that modify a condition or relationship characteristic of a feature or features of an embodiment of the present technology, are to be understood to mean that the condition or characteristic is defined to within tolerances that are acceptable for operation of the embodiment for an application for which it is intended, or within variations expected from the measurement being performed and/or from the measuring instrument being used. When the term “about” and “approximately” precedes a numerical value, it is intended to indicate +/−15%, or +/−10%, or even only +1-5%, and in some instances the precise value. Furthermore, unless otherwise stated, the terms (e.g., numbers) used in this disclosure, even without such adjectives, should be construed as having tolerances which may depart from the precise meaning of the relevant term but would enable the invention or the relevant portion thereof to operate and function as described, and as understood by a person skilled in the art.

While this disclosure has been described in terms of certain embodiments and generally associated methods, alterations and permutations of the embodiments and methods will be apparent to those skilled in the art. The present disclosure is to be understood as not limited by the specific embodiments described herein.

Certain marks referenced herein may be common law or registered trademarks of third parties. Use of these marks is by way of example and shall not be construed as descriptive or limit the scope of this disclosure to material associated only with such marks.

1. A hair styling composition for modifying a shape of mammalian hair fibers, the composition comprising a) at least one energy-curable water-insoluble phenol-based monomer (PBM) having an average molecular weight of 10,000 g/mol or less; b) at least one polar water-soluble hygroscopic agent (WHA); and c) water; wherein the hair styling composition is further characterized by one of more of the following features:
a—the hair styling composition contains less than 0.1 wt. % of small reactive aldehydes (SRA), the SRA being selected from formaldehyde, formaldehyde-forming chemicals, glutaraldehyde, and glutaraldehyde-forming chemicals;
b—the hair styling composition contains less than 1 wt. % of amino acids;
c—the hair styling composition contains less than 1 wt. % of peptides; and
d—the hair styling composition contains less than 1 wt. % of proteins.

2. The hair styling composition as claimed in claim 1, wherein the at least one energy-curable water-insoluble PBM is of Formula I: 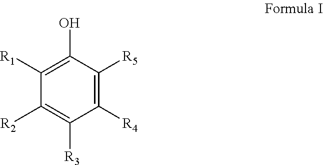
wherein:
i) R₁, R₂, R₃ and R₅ are each independently a hydrogen atom, a hydroxyl, or a linear, branched or cyclic, substituted or unsubstituted, C₁-C₂₀ alkyl, C₁-C₆ alkoxy, C₁-C₆ allyl, C₁-C₈ aromatic ester, or C₁-C₈ non-aromatic ester; and
ii) R₄ is a hydrogen atom, provided that R₁, R₂, R₃ and R₅ are not all hydrogen atoms, a hydroxyl, or a saturated or unsaturated C_{X}H_{Y} alkyl, wherein X is an integer equal to or less than 15, and Y is equal to 2X+1-n, n being selected from 0, 2, 4 and 6.

3. The hair styling composition as claimed in claim 2, wherein the at least one energy-curable water-insoluble PBM is of Formula VII: 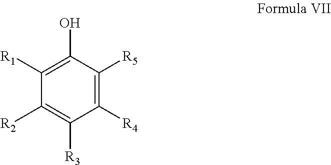
wherein:
i) at least one of R₁, R₂, and R₃ is a carboxylate substituent formed of a linear, branched or cyclic, substituted or unsubstituted, C₁-C₈ aromatic ester or C₁-C₈ non-aromatic ester, a non-carboxylate R₁, R₂, or R₃ being a hydrogen atom or a hydroxyl group; and
ii) R₄ and R₅ are each independently a hydrogen atom or a hydroxyl group.

4. The hair styling composition as claimed in claim 3, wherein the at least one energy-curable water-insoluble PBM is A) an ortho-hydroxybenzoic acid derivative wherein the carboxylate substituent is R₁, the PBM being a salicylate selected from a group comprising amyl salicylate, benzyl salicylate, 2-ethylhexyl salicylate, 4-tert-butylphenyl salicylate, cyclohexyl salicylate, methyl salicylate, hexyl salicylate, octyl salicylate, phenyl salicylate, salicin, and salsalate; B) a meta-hydroxybenzoic acid derivative wherein the carboxylate substituent is R₂, the PBM being selected from a group comprising methyl 3-hydroxybenzoate and phenyl 3-hydroxybenzoate; or C) a para-hydroxybenzoic acid derivative wherein the carboxylate substituent is R₃, the PBM being selected from a group comprising benzyl 4-hydroxybenzoate, butyl 4-hydroxybenzoate, ethyl 4-hydroxybenzoate, heptyl 4-hydroxy-benzoate, methyl 4-hydroxybenzoate, phenyl 4-hydroxybenzoate, isopropyl 4-hydroxy-benzoate and N-propyl 4-hydroxybenzoate.

5. The hair styling composition as claimed in claim 4, wherein the ortho-, meta- or para-hydroxybenzoic ring of the energy-curable water-insoluble PBM is further substituted by one or more hydroxyl groups, the relative positioning on the ring of the two or more hydroxyl groups with respect to the carboxylate substituent being selected from 2,3-dihydroxy-benzoates; 2,4-dihydroxy-benzoates; 2,5-dihydroxy-benzoates; 2,6-dihydroxy-benzoates; 3,4-dihydroxy-benzoates; 3,5-dihydroxy-benzoates; 2,3,4-trihydroxy-benzoates; 2,4,6-trihydroxy-benzoates, and 3,4,5-trihydroxybenzoates; and/or the carboxylate substituent is further substituted by an hydroxyl or an amine group.

6. The hair styling composition as claimed in claim 1, wherein the, or each, water-soluble hygroscopic agent is characterized by at least one, at least two, or at least three of the following structural features:
i) the WHA is non-electrolyte;
ii) the WHA has a hydrogen bond energy with water of at least 21 kJ/mol;
iii) the WHA has a hydrogen bond energy with water of at most 40 kJ/mol;
iv) the WHA has a hydrogen bond energy with water in a range between 21 kJ/mol and 40 kJ/mol;
v) the WHA has a solubility in water of 5 wt. % or more, by weight of the water, as measured at a temperature of 25° C.;
vi) the WHA has a solubility in water of 150 wt. % or less by weight of the water, as measured at a temperature of 25° C.;
vii) the WHA has a solubility in water in a range between 5 wt. % and 150 wt. % by weight of the water, as measured at a temperature of 25° C.;
viii) the WHA has a solubility in the hair styling composition or in an aqueous phase thereof of 5 wt. % or more by weight of the composition or aqueous phase thereof, as measured at a temperature of 25° C.;
ix) the WHA has a solubility in the hair styling composition or in an aqueous phase thereof of 140 wt. % or less by weight of the composition or aqueous phase thereof, as measured at a temperature of 25° C.;
x) the WHA has a solubility in the hair styling composition or in an aqueous phase thereof in a range between 5 wt. % and 140 wt. % by weight of the composition or aqueous phase thereof, as measured at a temperature of 25° C.;
xi) the WHA has a melting temperature (Tₘ) of 25° C. or more;
xii) the WHA has a melting temperature (Tₘ) of 200° C. or less;
xiii) the WHA has a melting temperature (Tₘ) in a range between 25° C. and 200° C.;
xiv) the WHA has a boiling temperature (T_{b}) of 100° C. or more;
xv) the WHA has a boiling temperature (T_{b}) of 300° C. or less;
xvi) the WHA has a boiling temperature (T_{b}) in a range between 100° C. and 300° C.;
xvii) the WHA has a vapor pressure of 2.3 kPa or less, as measured at 25° C.; and
xviii) the WHA has a vapor pressure of 1 mPa or more, as measured at 25° C.

7. The hair styling composition as claimed in claim 1, wherein each of the at least one WHA is selected from a group consisting of carboxamides, monosaccharides and disaccharides.

8. The hair styling composition as claimed in claim 7, wherein the at least one WHA is a carboxamide having the general formula RC(═O)NR′R″, wherein R, R′, and R″ each independently represent a linear, branched or cyclic, substituted or unsubstituted, organic group not exceeding 6 carbon atoms, or a hydrogen atom, said carboxamide being selected from a group consisting of urea, methanamide, ethanamide, propaneamide, butanamide; cyclopropane carboxamide, cyclobutane carboxamide, cyclopentane carboxamide; cyclohexane carboxamide; ethanediamide, propanediamide, butanediamide, pentanediamide, hexane-diamide; alanine amide, asparagine amide, glutamine amide, glycine amide, and proline amide.

9. The hair styling composition as claimed in claim 1, wherein:
A—a combined concentration of the at least one PBM is at least 0.1 wt. % by weight of the hair styling composition; and/or
B—a combined concentration of the at least one PBM is at most 5 wt. % by weight of the hair styling composition; and/or
C—a combined concentration of the at least one WHA is at least 10 wt. % by weight of the hair styling composition; and/or
D—a combined concentration of the at least one WHA is at most 50 wt. % by weight of the hair styling composition.

10. The hair styling composition as claimed in claim 1, further comprising at least one curing facilitator selected from a cross-linker and a curing accelerator, the curing facilitator being adapted to be in a same phase as the PBM within the hair fibers.

11. The hair styling composition as claimed in claim 10, wherein a combined concentration of the at least one curing facilitator is at least 0.001 wt. % and/or at most 5 wt. % by weight of the hair styling composition.

12. The hair styling composition as claimed in claim 10, wherein the at least one curing facilitator is at least one cross-linker selected from reactive silanes having at least two silanol groups and a molecular weight of at most 1,000 g/mol, mixtures of reactive silanes and amino-silanes, polybasic acids, polyols, polyamines, mono- and di-glycidyls, di-isocyanates, allylic compounds, polyphenols, acrylates, silsesquioxanes having organic glycidyl or methacrylate groups attached thereto and straight, branched or cyclic alkene compounds including up to fifteen carbon atoms, and containing a number of double bonds allowing for the formation of at least two radicals upon opening of the double bonds.

13. The hair styling composition as claimed in claim 10, wherein the at least one curing facilitator is a curing accelerator suitable for at least one of condensation polymerization and addition polymerization, the curing accelerator being selected from metal complexes, metal soaps, metal salen and organic peroxides.

14. The hair styling composition as claimed in claim 1, wherein the hair styling composition further comprises at least one auxiliary polymerization agent containing at least one functional group capable of cross-polymerization with at least one of the PBM and curing facilitator, the functional group being selected from: a hydroxyl, a carboxyl, an amine, an anhydride, an isocyanate, an isothiocyanate and a double bond.

15. The hair styling composition as claimed in claim 14, wherein the concentration of the auxiliary polymerization agent is between 0.01 wt. % and 2 wt. % by weight of the hair styling composition.

16. The hair styling composition as claimed in claim 1, wherein the composition further comprises at least one A—co-solvent selected from the group consisting of: C₁-C₁₀ alcohols having at least one hydroxyl group, water-miscible ethers, aprotic solvents, esters and mineral or vegetal oils; the co-solvent being in an amount controlling a form of the composition, the form of the hair styling composition being an oil-in-water emulsion or a single-phase composition; and/or B—additive selected from a group comprising an emulsifier, a wetting agent, a thickening agent and a charge modifying agent.

17. The hair styling composition as claimed in claim 1, wherein the hair styling composition has a glass transition temperature (Tg) of 50° C. or less.

18. The hair styling composition as claimed in claim 1, wherein the pH of the composition is different from the isoelectric point of the mammalian hair fibers to be treated therewith so as to enable penetration of at least a part of the PBM(s) and WHA(s) into the hair fibers.

19. A kit for styling mammalian hair fibers, the kit comprising:
i) a first compartment containing at least one energy-curable water-insoluble phenol-based monomer (PBM) having an average molecular weight of 10,000 g/mol or less; and
ii) a second compartment containing at least one polar water-soluble hygroscopic agent (WHA), and at least one of:
i) water;
ii) a co-solvent; and
iii) a pH modifying agent;
wherein the contents of the second compartment are a liquid having a pH selected to increase the penetration of at least part of the monomer into the hair fibers; and
wherein mixing of the compartments produces a single-phase composition or an oil-in-water emulsion constituting a hair styling composition for modifying a shape the mammalian hair fibers by application thereto;
the hair styling composition containing less than 0.1 wt. % of small reactive aldehydes (SRA), the SRA being selected from formaldehyde, formaldehyde-forming chemicals, glutaraldehyde, and glutaraldehyde-forming chemicals.

20. A method of styling mammalian hair fibers having a native shape, the method comprising:
a) applying to individual hair fibers a hair styling composition, the hair styling composition comprising at least one energy-curable water-insoluble phenol-based monomer (PBM) having an average molecular weight of 10,000 g/mol or less, at least one polar water-soluble hygroscopic agent (WHA) and water;
b) allowing the hair styling composition to remain in contact with the hair fibers for at least 5 minutes to ensure at least partial penetration of the PBM(s) and WHA(s) into the hair fibers; and
c) applying energy to at least partially cure at least part of the PBMs within the hair fibers, said curing occurring while the hair fibers are at a temperature of at least 50° C., so as to obtain treated hair fibers;
wherein the hair styling composition contains less than 0.1 wt. % of small reactive aldehydes (SRA), the SRA being selected from formaldehyde, formaldehyde-forming chemicals, glutaraldehyde, and glutaraldehyde-forming chemicals.
